(19) United States
(12) Patent Application Publication
(10) Pub. No.: US 2017/0216083 A1
(43) Pub. Date: Aug. 3, 2017

(54) SYSTEMS AND METHODS FOR TREATMENT OF SLEEP APNEA

(71) Applicants: Edward M. GILLIS, Livermore, CA (US); John H. SHADDUCK, Menlo Park, CA (US); Csaba TRUCKAI, Saratoga, CA (US); Andrew POUTIATINE, Mill Valley, CA (US); Paul J. BUSCEMI, Medina, MN (US); Octavian IANCEA, Sunnyvale, CA (US)

(72) Inventors: Edward M. GILLIS, Livermore, CA (US); John H. SHADDUCK, Menlo Park, CA (US); Csaba TRUCKAI, Saratoga, CA (US); Andrew POUTIATINE, Mill Valley, CA (US); Paul J. BUSCEMI, Medina, MN (US); Octavian IANCEA, Sunnyvale, CA (US)

(21) Appl. No.: 15/263,296

(22) Filed: Sep. 12, 2016

Related U.S. Application Data
(63) Continuation-in-part of application No. 14/674,986, filed on Mar. 31, 2015, now abandoned.
Application No. 14/674,986 is a continuation of application No. 13/711,537, filed on Dec. 11, 2012, now Pat. No. 8,991,398.
Application No. 13/711,537 is a continuation of application No. 13/269,520, filed on Oct. 7, 2011, now Pat. No. 8,327,854.
Application No. 13/269,520 is a continuation of application No. 12/937,564, filed on Jan. 3, 2011, now Pat. No. 8,707,960.
Application No. 12/937,564 is a continuation-in-part of application No. 15/198,826, filed on Jun. 30, 2016.
Application No. 15/198,826 is a continuation of application No. 14/289,475, filed on May 28, 2014, now Pat. No. 9,381,109.
Application No. 14/289,475 is a division of application No. 13/053,025, filed on Mar. 21, 2011, now Pat. No. 8,776,799.
Application No. 13/053,025 is a continuation-in-part of application No. 14/275,426, filed on May 12, 2014.
Application No. 14/275,426 is a division of application No. 13/053,059, filed on Mar. 21, 2011, now Pat. No. 8,733,363.
Application No. 13/053,059 is a continuation-in-part of application No. 14/877,862, filed on Oct. 7, 2015.
Application No. 14/877,862 is a continuation of application No. 13/113,933, filed on May 23, 2011, now abandoned.
Application No. 13/113,933 is a continuation-in-part of application No. 13/113,946, filed on May 23, 2011, now Pat. No. 9,510,922.
Application No. 13/113,946 is a continuation-in-part of application No. 13/188,385, filed on Jul. 21, 2011, now abandoned.
Application No. 13/188,385 is a continuation-in-part of application No. 13/308,449, filed on Nov. 30, 2011, now abandoned.
Application No. 13/308,449 is a continuation-in-part of application No. 13/311,460, filed on Dec. 5, 2011.
Application No. 13/311,460 is a continuation-in-part of application No. 13/539,081, filed on Jun. 29, 2012, now Pat. No. 9,439,801.
Application No. 13/539,081 is a continuation-in-part of application No. 13/935,052, filed on Jul. 3, 2013, now abandoned.
(60) Provisional application No. 61/052,586, filed on May 12, 2008.
(60) Provisional application No. 61/315,835, filed on Mar. 19, 2010.
(60) Provisional application No. 61/347,348, filed on May 21, 2010.
(60) Provisional application No. 61/315,838, filed on Mar. 19, 2010.
(60) Provisional application No. 61/347,356, filed on May 21, 2010.
(60) Provisional application No. 61/367,707, filed on Jul. 26, 2010.
(60) Provisional application No. 61/418,238, filed on Nov. 30, 2010.
(60) Provisional application No. 61/419,690, filed on Dec. 3, 2010.
(60) Provisional application No. 61/315,835, filed on Mar. 19, 2010.
(60) Provisional application No. 61/315,838, filed on Mar. 19, 2010.
(60) Provisional application No. 61/347,348, filed on May 21, 2010.
(60) Provisional application No. 61/347,356, filed on May 21, 2010.
(60) Provisional application No. 61/367,707, filed on Jul. 26, 2010.
(60) Provisional application No. 61/418,238, filed on Nov. 30, 2010.
(60) Provisional application No. 61/419,690, filed on Dec. 3, 2010.
(60) Provisional application No. 61/347,348, filed on May 21, 2010.
(60) Provisional application No. 61/347,356, filed on May 21, 2010.
(60) Provisional application No. 61/367,707, filed on Jul. 26, 2010.
(60) Provisional application No. 61/418,238, filed on Nov. 30, 2010.
(60) Provisional application No. 61/419,690, filed on Dec. 3, 2010.
(60) Provisional application No. 61/347,348, filed on May 21, 2010.
(60) Provisional application No. 61/347,356, filed on May 21, 2010.
(60) Provisional application No. 61/367,707, filed on Jul. 26, 2010.
(60) Provisional application No. 61/418,238, filed on Nov. 30, 2010.
(60) Provisional application No. 61/419,690, filed on Dec. 3, 2010.
(60) Provisional application No. 61/367,707, filed on Jul. 26, 2010.
(60) Provisional application No. 61/418,238, filed on Nov. 30, 2010.
(60) Provisional application No. 61/419,690, filed on Dec. 3, 2010.
(60) Provisional application No. 61/418,238, filed on Nov. 30, 2010.
(60) Provisional application No. 61/419,690, filed on Dec. 3, 2010.
(60) Provisional application No. 61/419,690, filed on Dec. 3, 2010.
(60) Provisional application No. 61/668,991, filed on Jul. 6, 2012.

(51) Int. Cl. A61F 5/56

(52) U.S. Cl. CPC A61F 5/566

(57) ABSTRACT

A method of maintaining airway patency in an airway of a patient. The method includes the steps of implanting a device into airway-forming tissue without affixing the device to the tissue and permitting a bioerodable portion of the device to bioerode to apply a force to the airway-forming tissue to maintain airway patency. The invention also provides devices for practicing the method.

### CROSS REFERENCE TO RELATED APPLICATIONS

The present application claims priority as a continuation-in-part of U.S. application Ser. No. 14/674,986, filed Mar. 31, 2015; which is a continuation of U.S. application Ser. No. 13/711,537, filed Dec. 11, 2012, now U.S. Pat. No. 8,991,398; which is a continuation of U.S. application Ser. No. 13/269,520, filed Oct. 7, 2011, now U.S. Pat. No. 8,327,854; which is a continuation of U.S. application Ser. No. 12/937,564, filed Jan. 3, 2011, now U.S. Pat. No. 8,707,960; which is a 371 of International Application No. PCT/US2009/043450, filed May 11, 2009; which claims the benefit of U.S. Provisional Patent Application No. 61/052,586, filed May 12, 2008.

The present application also claims priority as a continuation-in-part of U.S. application Ser. No. 15/198,826, filed Jun. 30, 2016; which is a continuation of U.S. application Ser. No. 14/289,475, filed May 28, 2014, now U.S. Pat. No. 9,381,109; which is a divisional of U.S. application Ser. No. 13/053,025, filed Mar. 21, 2011, now U.S. Pat. No. 8,776,799; which claims the benefit of U.S. Provisional Patent Application No. 61/315,835, filed Mar. 19, 2010; U.S. Provisional Patent Application No. 61/315,838, filed Mar. 19, 2010; U.S. Provisional Patent Application No. 61/347,348, filed May 21, 2010; U.S. Provisional Patent Application No. 61/347,356, filed May 21, 2010; U.S. Provisional Patent Application No. 61/367,707, filed Jul. 26, 2010; U.S. Provisional Patent Application No. 61/418,238, filed Nov. 30, 2010; and U.S. Provisional Patent Application No. 61/419,690, filed Dec. 3, 2010.

The present application also claims priority as a continuation-in-part of U.S. application Ser. No. 14/275,426, filed May 12, 2014; which is a divisional of U.S. application Ser. No. 13/053,059, filed Mar. 21, 2011, now U.S. Pat. No. 8,733,363; which claims the benefit of U.S. Provisional Patent Application No. 61/315,835, filed Mar. 19, 2010; U.S. Provisional Patent Application No. 61/315,838, filed Mar. 19, 2010; U.S. Provisional Patent Application No. 61/347,356, filed May 21, 2010; U.S. Provisional Patent Application No. 61/347,348, filed May 21, 2010; U.S. Provisional Patent Application No. 61/367,707, filed Jul. 26, 2010; U.S. Provisional Patent Application No. 61/418,238, filed Nov. 30, 2010; and U.S. Provisional Patent Application No. 61/419,690, filed Dec. 3, 2010.

The present application claims priority as a continuation-in-part of U.S. application Ser. No. 14/877,862, filed Oct. 7, 2015; which is a continuation of U.S. application Ser. No. 13/113,933, filed May 23, 2011, now abandoned; and claims the benefit of U.S. Provisional Application No. 61/347,348, filed May 21, 2010; U.S. Provisional Patent Application No. 61/347,356, filed May 21, 2010; U.S. Provisional Patent Application No. 61/367,707, filed Jul. 26, 2010; U.S. Provisional Patent Application No. 61/418,238, filed Nov. 30, 2010; and U.S. Provisional Patent Application No. 61/419,690, filed Dec. 3, 2010.

The present application claims priority as a continuation-in-part of U.S. application Ser. No. 13/113,946, filed May 23, 2011, which claims the benefit of U.S. Provisional Patent Application No. 61/347,348, filed May 21, 2010, U.S. Provisional Patent Application No. 61/347,356, filed May 21, 2010, U.S. Provisional Patent Application No. 61/367,707, filed Jul. 26, 2010, U.S. Provisional Patent Application No. 61/418,238, filed Nov. 30, 2010, and U.S. Provisional Patent Application No. 61/419,690, filed Dec. 3, 2010.

The present application claims priority as a continuation-in-part of U.S. application Ser. No. 13/188,385, filed Jul. 21, 2011; which claims the benefit of U.S. Provisional Patent Application No. 61/367,707, filed Jul. 26, 2010; U.S. Provisional Patent Application No. 61/418,238, filed Nov. 30, 2010; and U.S. Provisional Patent Application No. 61/419,690, filed Dec. 3, 2010.

The present application claims priority as a continuation-in-part of U.S. application Ser. No. 13/308,449, filed Nov. 30, 2011; which claims the benefit of U.S. Provisional Patent Application No. 61/418,238, filed Nov. 30, 2010; and U.S. Provisional Patent Application No. 61/419,690, filed Dec. 3, 2010.

The present application also claims priority as a continuation-in-part of U.S. application Ser. No. 13/311,460, filed Dec. 5, 2011; which claims the benefit of U.S. Provisional Patent Application No. 61/419,690, filed Dec. 3, 2010.

The present application also claims priority as a continuation-in-part of U.S. application Ser. No. 13/539,081, filed Jun. 29, 2012; and U.S. application Ser. No. 13/935,052, filed Jul. 3, 2013; which claims the benefit of U.S. Provisional Patent Application No. 61/668,991, filed Jul. 6, 2012.

### INCORPORATION BY REFERENCE

All publications, patents and patent applications mentioned in this specification are herein incorporated by reference to the same extent as if each individual publication, patent or patent application was specifically and individually indicated to be incorporated by reference.

### FIELD

The invention relates to the field of methods and devices for the treatment of obstructive sleep apnea, and more particularly to opening the airway of subjects with symptoms of obstructive sleep apnea.

### BACKGROUND

Sleep apnea is defined as the cessation of breathing for ten seconds or longer during sleep. During normal sleep, the throat muscles relax and the airway narrows. During the sleep of a subject with obstructive sleep apnea (OSA), the upper airway narrows significantly more than normal, and during an apneic event, undergoes a complete collapse that stops airflow. In response to a lack of airflow, the subject is awakened at least to a degree sufficient to reinitiate breathing. Apneic events and the associated arousals can occur up to hundreds of times per night, and become highly disruptive of sleep. Obstructive sleep apnea is commonly but not exclusively associated with a heavy body type, a consequence of which is a narrowed oropharyngeal airway.

Cyclic oxygen desaturation and fragmented sleeping patterns lead to daytime sleepiness, the hallmark symptom of the disorder. Further consequences of sleep apnea may include chronic headaches and depression, as well as diminished facilities such as vigilance, concentration, memory, executive function, and physical dexterity. Ultimately, sleep apnea is highly correlated with increased mortality and life threatening co-morbidities. Cardiology complications include hypertension, congestive heart failure, coronary artery disease, cardiac arrhythmias, and atrial fibrillation. OSA is a highly prevalent disease conditions in the United States. An estimated 18 million Americans suffer from OSA to degrees that range from mild to severe, many of whom are undiagnosed, at least in part because the afflicted subjects are often unaware of their own condition.

Treatment of OSA usually begins with suggested lifestyle changes, including weight loss and attention to sleeping habits (such as sleep position and pillow position), or the use of oral appliances that can be worn at night, and help position the tongue away from the back of the airway. More aggressive physical interventions include the use of breathing assist systems that provide a positive pressure to the airway through a mask that the subject wears, and which is connected to a breathing machine. In some cases, pharmaceutical interventions can be helpful, but they generally are directed toward countering daytime sleepiness, and do not address the root cause. Some surgical interventions are available, such as nasal surgeries, tonsillectomy and/or adenoidectomy, reductions in the soft palate or the uvula or the tongue base, or advancing the tongue base by an attachment to the mandible and pulling the base forward. These surgical approaches can be quite invasive and thus have a last-resort aspect to them, and further, simply do not reliably alleviate or cure the condition. There is a need for less invasive procedures that show promise for greater therapeutic reliability. There is additional need for the ability to reverse procedures or otherwise revise the procedure, thus allowing for the ability to reverse or otherwise revise the effects of the procedure due to side effects or other undesirable outcomes which may result from the procedure. Additionally, there is the need to do these procedural reversals or revisions in a manner that does not require excessive tissue cutting or invasiveness which can act as a deterrent for patients or physicians to perform such a revision procedure.

### SUMMARY OF THE DISCLOSURE

The invention relates to a method of alleviating obstructive collapse of airway-forming tissues, and for devices with which to implement the method. Typical patients for whom the method and device may provide therapeutic benefit are those who suffer from obstructive sleep apnea. The method includes implanting a device at a site in the tissue and bioeroding the bioerodible portion of the device to change the shape of the device and to remodel the airway-forming tissue. The implanted device is sized and shaped to conform to the airway-forming tissue site in a manner compatible with normal physiological function of the site; and includes a resiliently deformable portion and a bioerodible portion. In typical embodiments of the method, remodeling the airway-forming tissue results in the airway being unobstructed during sleep, and further, typically, the thus-unobstructed airway diminishes the frequency of apneic events. Remodeling may include reshaping or otherwise altering the position or conformation of airway associated tissue so that its tendency to collapse during sleep is diminished.

The airway is formed from various tissues along its length from the mouth to the lungs. Embodiments of the method include implanting an elastomeric implant or device into any one or more of these tissues, including, for example, the soft palate, the tongue, generally the base of the tongue, and the pharyngeal walls, typically the posterior and lateral portions of the pharyngeal wall.

In some embodiments, the device is in a deformed shape when implanted, and a bioerodable portion erodes to thereby release a tensioned shape of the implant to apply retraction forces to the site.

With regard to the bioeroding of the bioerodible portion of the device, this may occur over a time span that ranges from days to months. In some embodiments, the bioeroding proceeds at a rate that correlates with the ratio of the biologically-exposed surface area of the bioerodible portion to the volume of the bioerodible portion.

In some embodiments of the method, the bioerosion occurs at a rate that is sufficiently slow for the tissue site to recover from the implanting prior to the device substantially changing shape. In some of these embodiments, the recovery of the tissue site includes a forming of fibrotic tissue around the device, which typically stabilizes the device in the site, and provides the device greater leverage with which to reform the shape of the implant site and its surrounding tissue. In some embodiments, after implanting, and as part of the healing response or recovery from the implantation wound, the newly formed fibrotic tissues infiltrates into holes, pores, or interstices in the device. In some embodiments of the method, a bioactive agent, previously incorporated into the bioerodible material, is released or eluted from the bioerodible portion of the device as it is eroding.

In another aspect of the methods described herein, a method of forming a device to alleviate obstructive collapse of an airway during sleep is provided. The method includes forming a resiliently deformable material into an initial shape that corresponds to the preferred shape of the device, the initial shape having a site for accommodating bioerodible material; changing the initial shape of the resiliently deformable material into a non-preferred shape that is sized and configured into an implantable shape that conforms to an airway-forming tissue site and is compatible with normal physiological function after implantation; and stabilizing the implantable shape by incorporating the bioerodible material into the accommodating site. In some of these method embodiments, changing the initial shape of the resiliently deformable material includes absorbing a force sufficient to remodel the airway as the force is transferred from the device into an implant site after implantation of the device. That level of force is further typically insufficient to remodel the airway to an extent that it is unable to move in a manner that allows substantially normal or acceptable physiological function of the airway.

As noted above, the disclosure further provides a device for alleviating obstruction in an airway, such obstruction typically occurring during sleep. Embodiments of the device include an implantable device sized and shaped to conform to an airway-forming tissue site in a manner compatible with normal physiological function of the site, the device including a resiliently deformable portion and a bioerodible portion. In these embodiments, the resiliently deformable portion has a preferred shape that is constrained in a deformed shape by the bioerodible portion, and the device is configured to return toward the preferred shape of the resiliently deformable portion upon erosion of the bioerodible portion. In some embodiments, the preferred configuration is adapted to remodel the shape of the airway so as to provide a more open airway during sleep.

In typical embodiments of the device, the resiliently deformable portion may include any one or more of a metal or a polymer. In these embodiments, a resiliently deformable metal may include any one or more of stainless steel, spring steel, or superelastic nickel-titanium alloy, and a resiliently deformable polymer may include any one or more of silicon rubber, polyesters, polyurethanes, or polyolefins. In some embodiments, the bioerodible portion may include any one or more of polycaprolactone, polylactic acid, polyglycolic acid, polylactide coglycolide, polyglactin, poly-L-lactide, polyhydroxalkanoates, starch, cellulose, chitosan, or structural protein.

Some embodiments of the device include a portion adapted to engage the tissue into which it is implanted, and in some of these embodiments, the so-adapted portion includes a site for tissue in-growth, such in-growth serving to keep the device and tissue in close proximity, serving to promote implant site remodeling in a manner that conforms to the changing shape of the device. Finally, in some embodiments, the implantable device is configured with sufficient elasticity to allow normal physiological movement around an airway-forming tissue implant site when the device is implanted in the implant site.

In other embodiments, the adapted portion contains sites for tissue to link through the implant after implantation forming tissue plugs which thus form an attachment between the implant and the adjacent tissue without a corresponding adhesion of tissue to the implant. This type of arrangement can produce an implant that can effectively attach to and move tissue while remaining easily removable from the tissue. The tissue plugs can be formed by linking the implant around an encircled mass of tissue or allowing tissue to heal through the implant thus forming the island of encircled tissue. Implants can contain one or more encircled masses of tissue allowing attachment to the adjacent tissue. In some embodiments, a proximal end of the implant is anchored to the patient's mandible and a distal end or ends of the implant is/are releasably anchored to one or more tissue plugs.

The present invention provides methods and devices for treating obstructive sleep apnea. Embodiments of the invention include methods for opening a collapsed or obstructed airway with devices that can be implanted into various tissues that form the airway. Embodiments of the devices include resiliently deformable materials and bioerodable materials. The deformable portion of the devices is first formed into a preferred shape which is then subsequently deformed and stabilized in that deformed shape by incorporation or application of bioerodable materials to create a device in its implantable form. Once implanted into a tissue site, and thus exposed to an aqueous environment and to cellular and enzymatic action, the bioerodable portions of the device erode, thereby allowing the deformable portion of the device to return toward an at-rest form. Embodiments of the method, in their simplest form, thus include implanting a device, the bioerodable portion of the device bioeroding, the device changing shape as a consequence of the bioeroding, and the tissue remodeling in accordance with the force being exerted by the shape changing of the device.

One aspect of the invention provides a method of maintaining airway patency in an airway of a patient. The method includes the steps of implanting a device into airway-forming tissue without affixing the device to the tissue and permitting a bioerodable portion of the device to bioerode to apply a force to the airway-forming tissue to maintain airway patency. In some embodiments, the method also includes the step of expanding a portion of the device without affixing the device to the tissue, such as by, for example, permitting the portion of the device to self-expand. In various embodiments, the implanting step may include the step of inserting the device into the patient submandibularly, sublingually, and/or intra-orally.

In some embodiments, the permitting step includes the step of changing a shape of the device when the bioerodable portion bioerodes, such as by changing a length, curvature and/or width of the device. The method may also include the step of permitting newly formed tissue to infiltrate the device, possibly with the newly formed tissue at least partially infiltrating the device prior to applying a force to the airway-forming tissue.

In various embodiments, the implanting step includes the step of inserting the device into tongue tissue, soft palate tissue, pharyngeal wall tissue and/or epiglottis tissue. The method may also include the step of releasing a bioactive agent from the bioerodable portion as it bioerodes.

Another aspect of the invention provides a device for maintaining patency of an airway of a patient. In some embodiments, the device has a body having an at-rest shape and a deformed shape, the body being adapted to be implanted into airway-forming tissue of the patient, and proximal and distal anchors adapted to be implanted into the airway-forming tissue, without affixing the device to the tissue, and to be infiltrated by tissue to affix the anchors to the airway-forming tissue, with at least one bioerodable element maintaining the body in the deformed shape against a return force and the body being configured to return toward the at-rest shape upon erosion of the bioerodable element. In various embodiments, the body is sized and shaped to be inserted into tongue tissue, into soft palate tissue, and/or into pharyngeal tissue.

In various embodiments, the bioerodable element includes a coil and/or a C-shaped element. In some embodiments, at least one of the proximal and distal anchors is adapted to expand, possibly through self-expansion. One or more of the anchors may contain woven and/or non-woven material and may include through-holes to permit tissue in-growth. One or more of the anchors may also contain braided material.

In some embodiments, the device's deformed shape is longer, straighter and/or wider than its at-rest shape. The device may also have an elutable bioactive agent in some embodiments.

In some embodiments, a method of treating an airway disorder comprises implanting an axially-extending implant in an airway-interface tissue, the implant having first and second anchoring ends that are axially non-stretchable. In these embodiments, a medial implant portion is stretchable and configured to allow normal physiological function during non-sleep and to alleviate airway obstruction during sleep.

In some embodiments, a method of treating an airway disorder comprises implanting an axially-extending implant in a patient's tongue, the implant having first and second anchoring ends that are axially non-stretchable. In some of these embodiments, each end extends axially at least 15%, 20%, 25%, 30%, 35% or 40% of the overall axial length of the implant. In some of these embodiments, each end extends axially at least 4 mm, 6 mm, 8 mm, 10 mm or 12 mm.

In some embodiments, a method of treating an airway disorder comprises implanting an axially-extending implant in a patient's tongue, the implant having first and second anchoring ends and a medial portion therebetween. In these embodiments, the anchoring ends are axially non-stretchable. In some these embodiments, the implant medial portion is elastic and extends axially at least 40%, 50%, 60% or 70% of the overall axial length of the implant. In some of these embodiments, the implant medial portion is elastic and extends axially at least 10 mm, 12 mm, 14 mm or 16 mm in a repose state.

In some embodiments, methods of treating an airway disorder comprise introducing an introducer working end carrying a deployable implant into an airway-interface tissue. The implant has first and second anchoring ends. These methods include localizing an implant anchoring end within the tissue by observing light emission from an emitter location in the working end. The light emission may be provided by light propagating in a light channel extending to the working end. The light channel may comprise an optic fiber. The light emission may be provided by a light emitting diode (LED). The LED may be carried by the working end.

Some of the above methods further comprise deploying an anchoring end at a selected site identified by the light emission. The deploying step may include retracting the introducer working end contemporaneous with maintaining the anchoring end in the selected site. The maintaining step may be accomplished by maintaining an elongate element in contact with the implant end, with the element extending through the introducer working end. The maintaining step may be accomplished by penetrating a member through the airway-interface tissue to engage the implant end. In some methods, the airway-interface tissue comprises the tongue. In other methods, the airway-interface tissue comprises the soft palate.

In some embodiments, methods of treating an airway disorder comprise introducing an introducer working end carrying a deployable implant into an airway-interface tissue. The methods further comprise localizing an anchoring end of the implant in the tissue by observing a light emission from the implant. In some of these methods, the light emission is provided light propagation in a light channel in the implant. In some methods the light emission is provided light reflection by the implant. In some methods the light is transmitted to the implant by an optic fiber. In some methods the light is transmitted to the implant by a pusher member configured to deploy the implant from the working end.

In some embodiments, an implant for treating an obstructive airway disorder comprises an elongate body configured for implanting in an airway-interface tissue. In some of these embodiments, at least a portion of the elongate body carries a light guide for directing light transmission therethrough. In some embodiments, at least a portion of the elongate body carries a light reflective material for reflecting light transmission therein. In some embodiments, at least a portion of the elongate body carries a light transmission material for permitting light transmission therein.

In some embodiments, a system for treating an obstructive airway disorder comprises an elongate introducer carrying an implant configured for implanting in an airway-interface tissue. A light guide and/or a light emitter may be carried by the introducer. The elongate introducer may further comprise markings carried along its length configured for indicating the depth of penetration in tissue and further indicating the preferred implant length. The elongate introducer may be configured with a lumen for receiving an implant.

In some embodiments, a system for treating an obstructive airway disorder comprises an elongate member carrying a plurality of light emitters. The member is configured for insertion into airway-interface tissue. The light emitters may be spaced apart by predetermined dimensions to provide data to an observer for sizing an obstructive sleep apnea (OSA) implant.

In some embodiments, a system for treating an obstructive airway disorder comprises an elongate device extending along an axis configured for insertion into airway-interface tissue. The device comprises first and second axially translatable elements for moving first and second light emitters axially relative to one another. The elongate device may further be configured to carry a deployable OSA implant.

In some embodiments, a method of treating an airway disorder comprises introducing an elongate element into an airway-interface tissue. The element carries at least two locations for providing light emissions. The method also comprises observing light emission from the at least two locations to thereby determine target sites for anchoring ends of an implant. The method further comprises selecting and deploying an implant with its anchoring ends in the target sites. The observing step may include adjusting the dimension between the light emission locations to determine suitable implant length. In some embodiments, the airway-interface tissue comprises the tongue. In some embodiments, the airway-interface tissue comprises the soft palate.

In some embodiments, a method of treating an airway disorder comprises inserting an axial-extending introducer into an airway-interface tissue. The introducer has markings along its axis to indicate depth penetration, and a light emitter at a distal end thereof. The method also comprises observing light emission from the distal end and observing depth of penetration. The method further comprises selecting an implant length based on the observations and implanting the implant through a lumen in the introducer. In some embodiments, the airway-interface tissue comprises the tongue. In some embodiments, the airway-interface tissue comprises the soft palate.

Another aspect of the invention provides an implant system for implanting in airway forming tissue including a bioerodable material and an elongate long term implant, the bioerodable material at least partially enveloping the elongate long term implant and linked between a first set of two points on the bioerodable material to form a first bridge. In some embodiments, the bioerodable material is configured to hold the elongate long term implant in an initial shape (e.g. a tensioned state).

In some embodiments the bioerodable material includes a spring having at least two coils and a plurality of points, and the first bridge connects two points on the spring. In some embodiments, the two points are on different coils at a first end of the spring. In some embodiments, the bioerodable material is linked between a second set of two points on a second set of coils of the spring to form a second bridge. In some of these embodiments the second set of coils is at a second end of the spring. In some of these embodiments, the first and second bridges are bioerodable.

In some embodiments, substantially each coil is linked to at least one other coil to form a plurality of bridges.

Yet another aspect of the invention provides a resilient elongate implant body having a first insertion shape and a second therapeutic shape, and a bioerodable material having two coils that at least partially envelop the resilient elongate implant body, and the coils are coupled together to form a coupled coil structure. In some embodiments, the bioerodable material is configured to hold the implant body in the initial insertion shape. In some of these embodiments, the bioerodable material includes additional coils continuous with the two coils to form a spring and the additional coils of the spring are wrapped around the implant body, and the two coils are at an end of the spring. In some of these embodiments, substantially each coil is coupled to at least one other coil.

Yet another aspect of the invention provides a method of manufacturing an implant system, the implant having an elongate implant body and a bioerodable support material configured to hold the elongate implant body in a first, elongate shape, the method including the steps of wrapping the bioerodable support material at least partway around the implant body, the bioerodable support material having two points on it, and coupling the two points with each other to create a coupled bioerodable support material.

Yet another aspect of the invention includes a method of manufacturing a bioerodable implant including the steps of wrapping a bioerodable material at least partway around an axis to create a wound bioerodable implant, the bioerodable material including two points, and coupling the two points to each other. In some embodiments, the wound bioerodable implant includes a helix and coupling includes heating the helix to fuse the two points.

In some embodiments, the axis includes an elongate long term implant, and the wrapping around an axis comprises wrapping the bioerodable material around the elongate long term implant to create an implant system. In some of these embodiments, the method includes applying an expansive force to the elongate long term implant with the bioerodable material to hold the long term implant in an initial shape.

In some embodiments, the coupling step includes attaching a bioerodable material to the two points to create a support strut. In some embodiments, the coupling step includes applying at least one of an adhesive, an other chemical, or an energy source to the bioerodable material. In some embodiments, the coupling step includes heating the bioerodable material to melt the two points together.

### BRIEF DESCRIPTION OF THE DRAWINGS

FIG. 1 provides an overview of the healthy human airway anatomy, with particular attention to the nasopharyngeal, oropharangeal, and hypopharyngeal regions.

FIG. 2A provides a view of a compromised airway, with an occlusion in the oropharyngeal region due to posterior slippage of the base of the tongue.

FIG. 2B provides a view of a compromised airway with palate closure.

FIG. 3A depicts an elongate implant component of a revisable OSA implant system, the implant having end portions with openings for growth of a tissue plug therethrough to secure the end portions in a treatment site.

FIG. 3B is a cut-away view of an end portion of the implant of FIG. 3A in a tissue site.

FIG. 3C depicts another elongate implant embodiment similar to that of FIG. 3A.

FIG. 3D depicts another elongate implant embodiment.

FIG. 4 depicts another elongate implant corresponding to aspects of the invention.

FIG. 5A depicts a second component of a revisable OSA implant system, the second component comprising a cutting tool.

FIG. 5B depicts the cutting tool of FIG. 5A in a method of use.

FIG. 6 depicts an alternative cutting tool similar to that of FIGS. 5A-5B.

FIG. 7A depicts another elongate implant corresponding to aspects of the invention.

FIG. 7B depicts another elongate implant embodiment.

FIG. 7C depicts another elongate implant embodiment.

FIG. 7D depicts another elongate implant embodiment with multiple openings in multiple planes.

FIG. 7E depicts an OSA implant with an elastomeric portion that is configured for being releasably maintained in a tensioned or non-repose condition by a magnesium biodissolvable material or element.

FIG. 8A depicts the working end of another embodiment of a cutting tool for cutting a portion of an implant in situ.

FIG. 8B depicts another embodiment of a cutting tool for cutting an implant in a revision procedure.

FIG. 9 depicts another implant with a medial portion having a surface configured for low adhesive energy.

FIG. 10 depicts another elongate implant corresponding to aspects of the invention.

FIG. 11 depicts another implant corresponding to aspects of the invention including a sacrificial portion that can be sacrificed in response to an external stimulus.

FIG. 12 is a cut-away view depicting the implant of FIG. 11 in a tissue site after actuation of the sacrificial portion of the implant.

FIG. 13A depicts an alternative implant including an electrolytically sacrificial portion that can be sacrificed in response to a direct current.

FIG. 13B is a cut-away view depicting the implant of FIG. 13A in a tissue site after actuation of electrolytic connection portion of the implant.

FIG. 14 depicts an end portion of an alternative revisable implant including a cut wire for cutting a tissue plug.

FIG. 15 is a cut-away view depicting the implant of FIG. 14 in a tissue site in the process of actuating the cut wire.

FIG. 16 depicts an end portion of an alternative revisable implant including a cut wire for cutting a plurality of tissue plugs.

FIG. 17 depicts an alternative revisable OSA implant.

FIGS. 18A and 18B illustrate an end portion of the revisable implant of FIG. 17.

FIG. 19 depicts an alternative revisable OSA implant.

FIG. 20 depicts a revisable OSA implant that allows for in-situ post-implant adjustment of the retraction forces applied to tissue by the implant.

FIG. 21 depicts an alternative revisable OSA implant that allows for in-situ post-implant adjustment of the retraction forces.

FIGS. 22 and 23 depict another revisable OSA implant that allows for in-situ post-implant adjustment of the retraction forces.

FIG. 24 depicts an OSA implant with first and second anchoring ends implanted in a particular site in a patient's tongue.

FIG. 25 depicts the OSA implant of FIG. 24 implanted in another particular site in a patient's tongue.

FIGS. 26-27 depict a plurality of OSA implants each with first and second anchoring ends implanted in a patient's tongue for applying linear-directed forces in different distinct vectors.

FIGS. 28A, 28B and 28C depict another OSA implant system for applying linear-directed forces in different distinct vectors with individual implant bodies coupled together in-situ with attachment means.

FIGS. 29A-29B depict another OSA implant system similar to that of FIGS. 28A-28C for applying linear-directed forces in different distinct vectors in a different orientation.

FIG. 30 illustrates a method of utilizing a cannula apparatus for deployment of an OSA implant as in FIG. 24 in a particular site in a patient's tongue.

FIG. 31 illustrates a working end of the cannula apparatus of FIG. 30 together with a push rod or stylet mechanism for deployment of the OSA implant of FIG. 24.

FIGS. 32A-32B illustrate a method of utilizing an alternative telescoping cannula apparatus for deployment of an OSA implant at a selected angle in a patient's tongue.

FIG. 33 illustrates another method of utilizing a cannula apparatus to penetrate through a patient's skin for deployment of an OSA implant in a patient's tongue.

FIG. 34 illustrates another method of utilizing a curved cannula apparatus for deployment of an OSA implant in a patient's tongue.

FIG. 35A depicts another OSA implant that comprises a unitary V-shaped implant body with first and second legs and anchoring ends implanted in a patient's tongue for applying linear-directed forces in different distinct vectors.

FIG. 35B depicts first and second OSA implants that utilize a fibrotic response to effectively create in-situ a V-type implant with first and second legs for applying linear-directed forces in different vectors.

FIG. 36 depicts another OSA implant that is configured with an element of an anchoring end portion configured for extending transverse to the axis of contractile muscle fibers.

FIG. 37 illustrates another OSA implant that includes an elongated elastic portion and cooperating elongated bioerodible portion for temporarily maintaining the implant in an extended, stressed position.

FIG. 38A illustrates an OSA implant that has a curved configuration that can allow the tongue to move by straightening the implant.

FIG. 38B depicts the curved implant of FIG. 38A in a straightened shape with the tongue displaced posteriorly toward obstructing the airway.

FIG. 39 depicts a curved implant as in FIG. 38A implanted in a horizontal plane in the patient's tongue.

FIG. 40A depicts an S-shaped or serpentine implant in a vertical orientation that may allow the tongue to move by straightening the elastic implant.

FIG. 40B depicts the serpentine implant of FIG. 40A in a straightened shape with tongue displaced posteriorly.

FIG. 41 depicts a helical curved implant that again can allow the tongue to move by straightening the implant.

FIG. 42 depicts another type of implant that comprises a loop or encircling OSA implant with a connection means adjacent first and second ends thereof, the implant in a vertical orientation in a patient's tongue.

FIG. 43 depicts an encircling implant as in of FIG. 41 in horizontal orientation in a patient's tongue.

FIG. 44A depicts a device configured for implanting the encircling implant of FIGS. 42-43, with first and second trocar elements and a guide block.

FIGS. 44B-44E depict schematically the steps of using the working end of the device of FIG. 44A to implant and deploy an encircling implant in tissue.

FIGS. 44F-44G depict an encircling implant fully bridged between first and second trocars; FIG. 44G depicts the trocar system proximate the patient with the trocars being withdrawn, leaving the implant in place.

FIG. 44H depicts the final step of the method comprising fixedly connecting the two ends of the implant so as to form a loop or encircling implant.

FIG. 45 depicts various shapes of loop or encircling implants.

FIG. 46 depicts a loop or encircling implant with its ends fixedly connected around the geniohyoid muscle to serve as an anchor.

FIG. 47 depicts a U- or V-shaped implant with two anchors in the anterior position, adjacent to the mandible.

FIG. 48 illustrates a V-shaped implant with two anchors at the distal ends that are the legs of the V-shape in a horizontal orientation in a patient's tongue.

FIG. 49 illustrates a V-shaped implant with two anchors at the distal ends that are the legs of the V-shape in a vertical orientation in a patient's tongue.

FIG. 50A depicts a device and first step of a method for implanting the V-shaped implant of FIG. 48 in a patient's tongue, wherein two curved tunnelers form pockets for the legs of the V-shaped implant.

FIG. 50B depicts a subsequent step of the method wherein the tunnelers are removed, and two curved push rods with hooks at the distal ends thereof pushing or maintain the anchor ends of the implant in place.

FIG. 50C depicts the patient's tongue after the trocar is withdrawn leaving the V-shaped implant in its final position.

FIG. 51 depicts a V-shaped implant as in FIG. 50C anchored around the geniohyoid muscle.

FIG. 52 depicts a combination implant with an encircling portion anchored around the geniohyoid muscle and a linear portion with an anchoring end near the tongue base.

FIG. 53 depicts an elongated implant body having an elastomeric medial portion and a large planar end implanted in a tongue.

FIG. 54 depicts an elongated implant body having an intermediate release mechanism in the medial portion.

FIG. 55A illustrates two elongated implants in a patient's tongue wherein the implant orientations are non-parallel.

FIG. 55B illustrates a different view of the two elongated implants of FIG. 55A from a different perspective.

FIG. 56 illustrates two elongated implants in a patient's tongue wherein the implant orientations are asymmetric relative to the patient's mid-line.

FIG. 57 illustrates two elongated implants in a patient's soft palate wherein the implant orientations are parallel and symmetric relative to the patient's mid-line.

FIG. 58 illustrates two elongated implants in a patient's soft palate wherein the implant's axes converge in the posterior direction.

FIG. 59 illustrates two elongated implants in a patient's soft palate wherein the implant's axes diverge in the posterior direction.

FIG. 60 illustrates two elongated implants in a patient's soft palate wherein the implant's axes parallel and angled relative to the patient's mid-line.

FIG. 61 illustrates two elongated implants in a patient's soft palate wherein the implant's axes cross about the patient's mid-line.

FIG. 62 depict an implant configuration in which an elongated implant has a posterior portion that extends through the median raphe of the tongue.

FIGS. 63A-C show an airway-maintaining device according to one embodiment of the invention.

FIGS. 64A-B show an airway-maintaining device according to another embodiment of the invention. FIG. 64B is an enlarged cross-section along the lines shown in FIG. 64A.

FIGS. 64C-D show an airway-maintaining device according to yet another embodiment of the invention. FIG. 64D is an enlarged cross-section along the lines shown in FIG. 64C.

FIGS. 64E-F show an airway-maintaining device according to still another embodiment of the invention. FIG. 64F is an enlarged cross-section along the lines shown in FIG. 64E.

FIGS. 64G-H show an airway-maintaining device according to another embodiment of the invention. FIG. 64H is an enlarged cross-section along the lines shown in FIG. 64G.

FIGS. 64I-J show an airway-maintaining device according to yet another embodiment of the invention. FIG. 64J is a cross-section along the lines shown in FIG. 64I.

FIGS. 65A-C show implantation and use of an airway-maintaining device delivered submandibularly.

FIGS. 66A-C show implantation and use of an airway-maintaining device delivered intraorally and sublingually.

FIGS. 67A-C show implantation and use of an airway-maintaining device delivered intraorally to the soft palate.

FIGS. 68A-C show details of the device shown in FIG. 67.

FIGS. 69A-B show details of the device shown in FIGS. 67 and 68 in place in the soft palate.

FIGS. 70A-B show an airway maintaining device according to yet another embodiment of the invention in place in the patient.

FIG. 71 is a graph comparing tensile force applied by embodiments of the invention and theoretical force applied by other obstructive sleep apnea therapy devices.

FIGS. 72A-C show an airway-maintaining device according to still another embodiment of the invention.

FIGS. 73A-B show the device of FIG. 72 in place in patient.

FIGS. 74A-B show the devices of FIGS. 66 and 67 in place in a patient.

FIGS. 75A-C show multiple devices of FIGS. 66 and 67 in place in a patient.

FIGS. 76A-C show another embodiment of the airway maintaining device of this invention.

FIGS. 77A and 77B depict another OSA implant that allows for in-situ post-implant adjustment of the retraction forces.

FIG. 77C depicts another OSA implant with an elongate, linear fluid-tight chamber therein.

FIGS. 78A and 78B depict another OSA implant with a fluid-tight chamber configured for altering fluid volumes therein to adjust retraction forces applied by the implant.

FIG. 79 depicts another OSA implant with an elongate, non-linear fluid-tight chamber therein.

FIG. 80 depicts another OSA implant with an elongate, fluid-tight chamber therein with a sacrificial port.

FIG. 81 depicts an OSA implant with a plurality of fluid-tight chambers therein with sacrificial ports.

FIG. 82 depicts another OSA implant with a plurality of fluid-tight chambers therein with sacrificial ports.

FIG. 83 depicts an OSA implant with a fluid-filled chamber surrounded at least in part by a fluid-permeable wall.

FIGS. 84A and 84B depict an OSA implant with a heat shrink polymer material therein to adjust retraction forces applied by the implant.

FIG. 85 depicts an OSA implant with a shape memory polymer material therein to adjust retraction forces applied by the implant.

FIG. 86 depicts an OSA implant with tooth and ratchet mechanism to adjust retraction forces applied by the implant.

FIGS. 87A and 87B depict an OSA implant with a shape memory alloy frangibolt mechanism therein to adjust retraction forces applied by the implant.

FIGS. 88A-88B depict other embodiments of implant bodies configured with axially inelastic anchoring end portions and an elastic medial portion, wherein the end portions have a substantial axial length relative to the medial portions.

FIG. 89 is an enlarged view of an anchoring end of the implant body of FIG. 88B depicting non-stretchable interior elements.

FIG. 90 depicts an implant of FIG. 88A or FIG. 88B configured with axially inelastic anchoring end portions and an elastic medial portion implanted in a particular site in a patient's tongue.

FIG. 91 depicts multiple implants as shown in FIG. 88 configured with axially inelastic anchoring end portions and elastic medial portions implanted in a particular site in a patient's tongue.

FIG. 92 depicts a system for implanting an OSA implant wherein the introducer carries a light emitter for emitting an observable light for localizing an implant end in tissue.

FIG. 93 depicts another system for implanting an OSA implant wherein a telescoping introducer carries first and second light emitters for localizing both ends of an implant in tissue.

FIG. 94 depicts another system for implanting an OSA implant wherein an introducer sleeve carries a plurality of light emitters for determining an optimal length of an implant.

FIG. 95 depicts a method of using a system for implanting an OSA implant with an introducer sleeve that carries at least one light emitter.

FIG. 96 is an enlarged schematic view of an OSA implant that carries a light guide.

FIG. 97 depicts a method of using the system for implanting an OSA implant of the type shown in FIG. 96.

FIG. 98 shows a method of using a system for implanting an OSA implant as in FIG. 93 in soft palate tissue.

FIGS. 99 A-B show two views of an embodiment of a device or implant system with a bioerodable material around an elongate long-term implant.

FIGS. 99 C-D show two views of a device, such as that shown in FIGS. 99 A-B, after foreshortening.

FIGS. 100A-B show two views of a device that has prematurely foreshortened.

FIG. 101 shows a method of coupling a bioerodable material to itself to form a bridge according to one aspect of the invention.

FIGS. 102 A-C show two versions of a device with (FIGS. 102 A-B) and without (FIG. 102 C) coupling.

FIG. 103 A, B shows two views of a device with cuff or C-shaped portion partially enveloping a long term implant portion and connected by a bridge.

FIG. 104 shows an implant system with a modular design.

FIG. 105 shows a ribbon-like structure wrapped around a long term implant and coupled to itself.

FIG. 106 shows an embodiment of a device with a self-linking bioerodable portion.

FIG. 107 shows an embodiment of a device with a stent-like structure holding the long term portion.

FIGS. 108 and 109 show embodiments of devices with regions of greater flexibility and regions of lesser flexibility.

FIG. 110 shows an embodiment of a device with minimal wrapping of the bioerodable portion around the long term implant portion.

FIG. 111 shows another embodiment of a device with a single spring coupled to itself.

FIG. 112 shows another embodiment of a device according to the invention with the bioerodable portion coupled with the long-term elongate implant portion.

FIG. 113 depicts an end portion of an alternative revisable implant including a cut wire for cutting a tissue plug.

FIG. 114 is a cut-away view depicting the implant of FIG. 113 in a tissue site in the process of actuating the cut wire.

FIG. 115 depicts an end portion of an alternative revisable implant including a cut wire for cutting a plurality of tissue plugs.

FIGS. 116A-116B depict other embodiments of implant bodies configured with axially inelastic anchoring end portions and an elastic medial portion, wherein the end portions have a substantial axial length relative to the medial portions.

FIG. 117 is an enlarged view of an anchoring end of the implant body of FIG. 116B depicting non-stretchable interior elements.

FIGS. 118 A-D show implant length over time after implant placement in the tongue or soft palate.

### DETAILED DESCRIPTION

### A. Anatomy of the Pharynx

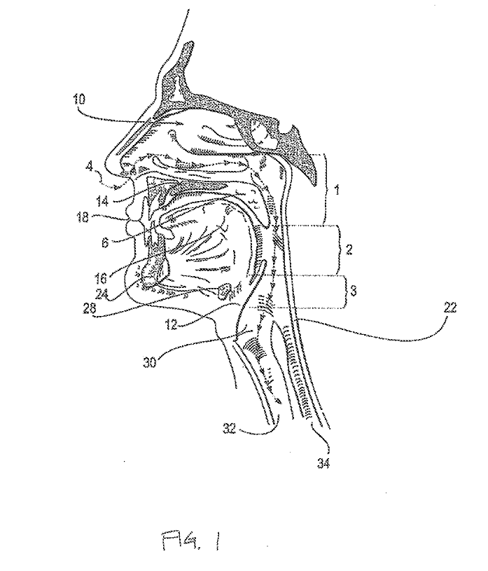

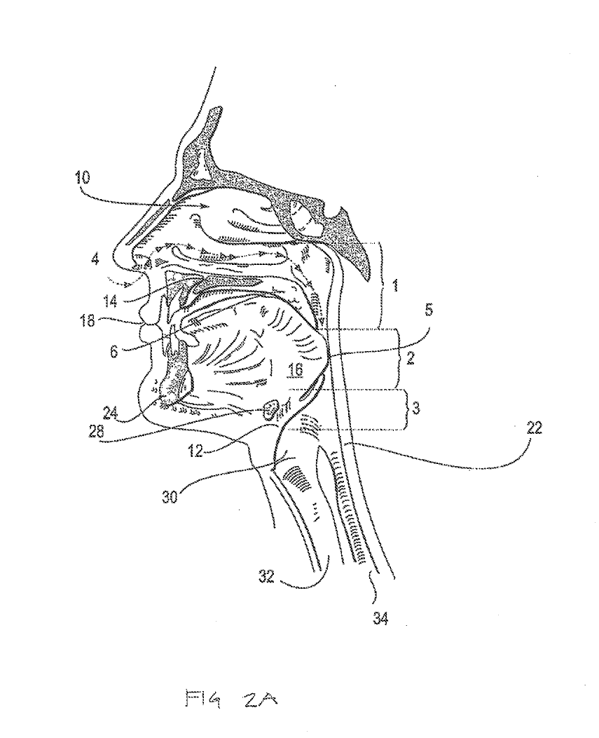

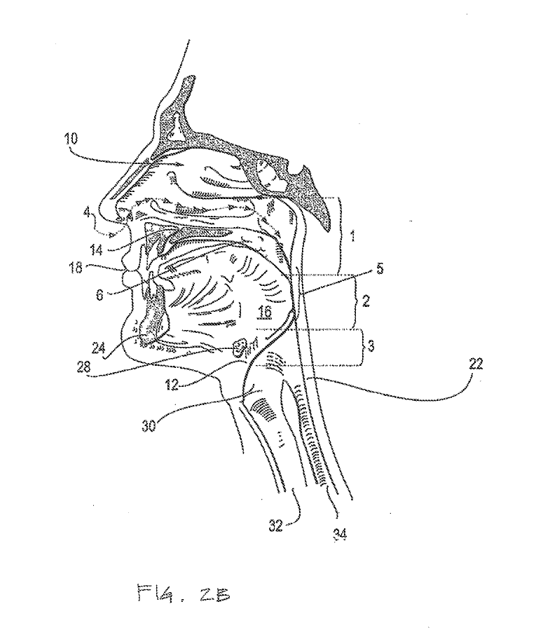

FIG. 1 is a sagittal view of the structures that form the pharyngeal airway 4; some of these structures can become compromised under various conditions to the extent that they obstruct or occlude passage of air through the airway 4, and thus contribute to obstructive sleep apnea. The pharynx is divided, from superior to inferior, into the nasopharynx 1, the oropharynx 2 and the hypopharynx 3. Variations of FIG. 1 are provided in FIGS. 2A and 2B which depict airway obstruction sites 5 at various levels in the pharyngeal airway. FIG. 2A, for example, shows an occlusion 5 at the level of the oropharynx 2, where the base of the tongue 16 and a thickened posterior pharyngeal wall 22 have collapsed against each other. FIG. 2B provides a view of a compromised airway with palate closure. It is also possible for airway obstruction to occur at the level of the nasopharynx 1, where an elongated and/or floppy soft palate can collapse against a thickened posterior pharyngeal wall. Further, an obstruction can occur at the level of the hypopharynx 3, where both an elongated soft palate and a floppy epiglottis can collapse against the pharyngeal wall 22.

With reference to FIGS. 1-2B, the nasopharynx is the portion of the pharynx at the level or above the soft palate 6. In the nasopharynx, a deviated nasal septum or enlarged nasal turbinates may occasionally contribute to upper airway resistance or blockage. Rarely, a nasal mass, such as a polyp, cyst or tumor may be a source of obstruction. The oropharynx 2 includes structures from the soft palate 6 to the upper border of the epiglottis 12 and includes the inferior surface of the hard palate 14, tongue 16, the posterior pharyngeal wall 22 and the mandible 24 as well as the tonsils and palatoglossal arch. The mandible typically has a bone thickness of about 5 mm to about 10 mm anteriorly with similar thicknesses laterally. An obstruction in the oropharynx 2 may result when the tongue 16 is displaced posteriorly during sleep as a consequence of reduced muscle activity during deep or non-REM sleep. The displaced tongue 16 may push the soft palate 6 posteriorly and may seal off the nasopharynx 1 from the oropharynx 2. The tongue 16 may also contact the posterior pharyngeal wall 22, which causes further airway obstruction.

The hypopharynx 3 includes the region from the upper border of the epiglottis 12 to the inferior border of the cricoid cartilage. The hypopharynx 3 further includes the hyoid bone 28, a U-shaped, free-floating bone that does not articulate with any other bone. The hyoid bone 28 is attached to surrounding structures by various muscles and connective tissues. The hyoid bone 28 lies inferior to the tongue 16 and superior to the thyroid cartilage 30. A thyrohyoid membrane and a thyrohyoid muscle attaches to the inferior border of the hyoid 28 and the superior border of the thyroid cartilage 30. The epiglottis 12 is infero-posterior to the hyoid bone 28 and attaches to the hyoid bone by a median hyoepiglottic ligament. The hyoid bone attaches anteriorly to the infero-posterior aspect of the mandible 24 by the geniohyoid muscle.

### B. Revisable OSA Implants

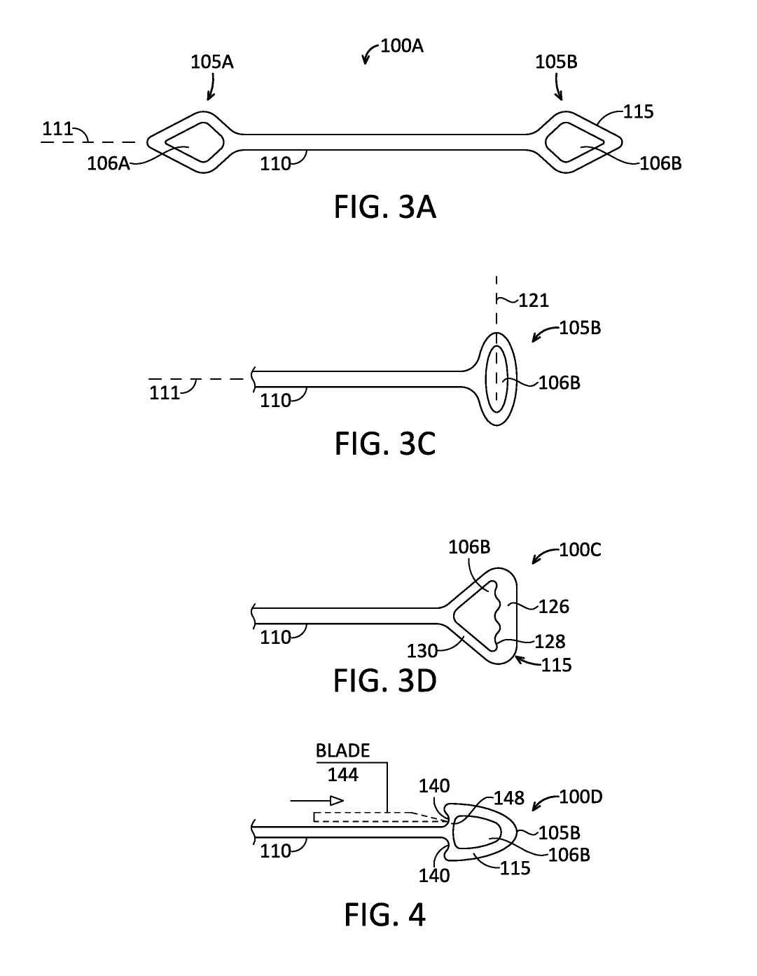

FIG. 3A depicts a first component of a kit or system that provides revisable implants for treating an airway disorders or obstructive sleep apnea (OSA). The second component of the kit is an introducer for insertion into a treatment site as is known in the art and co-pending applications. In FIG. 3A, an elongate device or implant body 100A has first and second end portions 105A and 105B with through-openings 106A and 106B therein. The medial portion 110 of the implant body 100A extends along axis 111 and comprises a biocompatible elastomeric material such as a silicone. The mean cross-section of the medial body portion 110 can range from 1 to 10 mm² and can be round, oval flat or polygonal. The elastic modulus of the medial portion can range from 0.5 to 10 MPA and is configured for implanting in the patient's airway tissue in a releasable, tensioned position, as described in co-pending U.S. patent application Ser. No. 11/969,201, which is incorporated herein by this reference.

Referring to FIGS. 3A and 3B, it can be seen that through-openings 106A and 106B in the implant body 100 are configured for growth of a tissue plug 112 through the opening to thereby secure the first and second end portions 105A and 105B in a selected tissue site. The cut-away view of FIG. 3B schematically illustrates that a tissue plug 112 that grows through the opening is thus surrounded or encircled by an encircling body portion 115 of the implant. The encircling body portion 115 comprises a small cross-section element that can be cut, severed, sacrificed, decoupled, or dissolved to disengage the implant from a tissue site 120 as will be described below. The element can be a polymer or other material. In other embodiments described below, the tissue plug 112 can be cut or severed to disengage the implant from the tissue site 120. In one embodiment, the mean cross-section of the tissue plug 112, and thus the dimension across an opening 106A or 106B, can range from about 0.5 mm to 10 mm or more. The openings 106A or 106B can have a round shape in plan view or any other plan shape. The end portions 105A and 105B can have similar or dissimilar configurations, for example an implant configured for treatment of a patient's tongue may have a substantially larger end portion and opening 106B for the base of the tongue and a smaller end portion near the mandible.

FIG. 3C illustrates another implant body 100B with an end portion 105B having an elongated opening 106B through which tissue will grow to form a tissue plug to secure the end portion in the site. For example, the implant body 100B of FIG. 3C has an opening 106B with a primary axis 121 and larger dimension that extends generally orthogonal to the axis 111 of medial portion 110 of the implant body. In use, the greater dimension of the tissue plug will better resist the retraction forces applied to tissue by the elastomeric medial portion 110 of the implant aligned with axis 111.

FIG. 3D depicts another embodiment 100C of a revisable implant for treating an airway disorder that is similar to that of FIG. 3C except the end portion 105B has a through-opening 106B with a terminal part 126 of encircling portion 115 configured with irregular shaped surface features 128 that can interface with the tissue plug that grows through opening 106B. The surface features can comprise undulations, textures, protrusions, bumps and the like that can assist in maintaining the end portion in a fixed position when under the tensioning or retraction forces applied by the medial portion 110 of the implant body 100C. In the implant body 100C of FIG. 3D, the end portion 105B also can have an encircling element 115 that includes a proximal portion 130 of a lower modulus material similar to the modulus of medial portion 110 and the terminal part 126 having a higher modulus to prevent it deformation under tensioning forces.

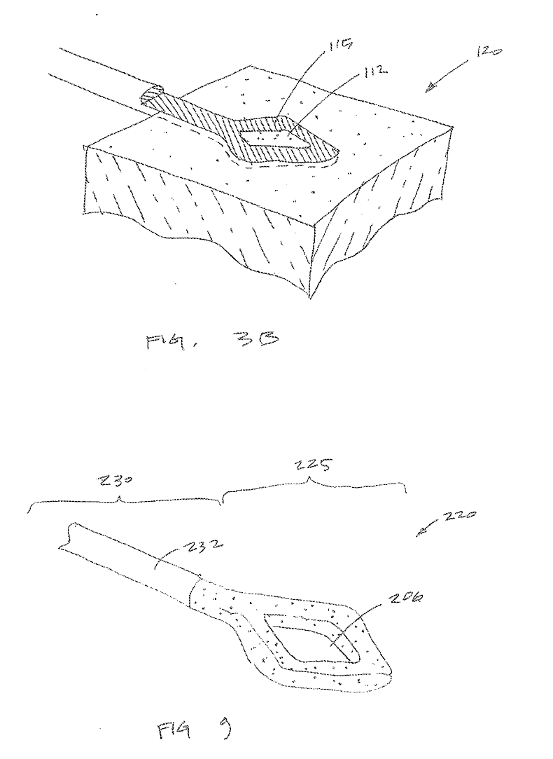

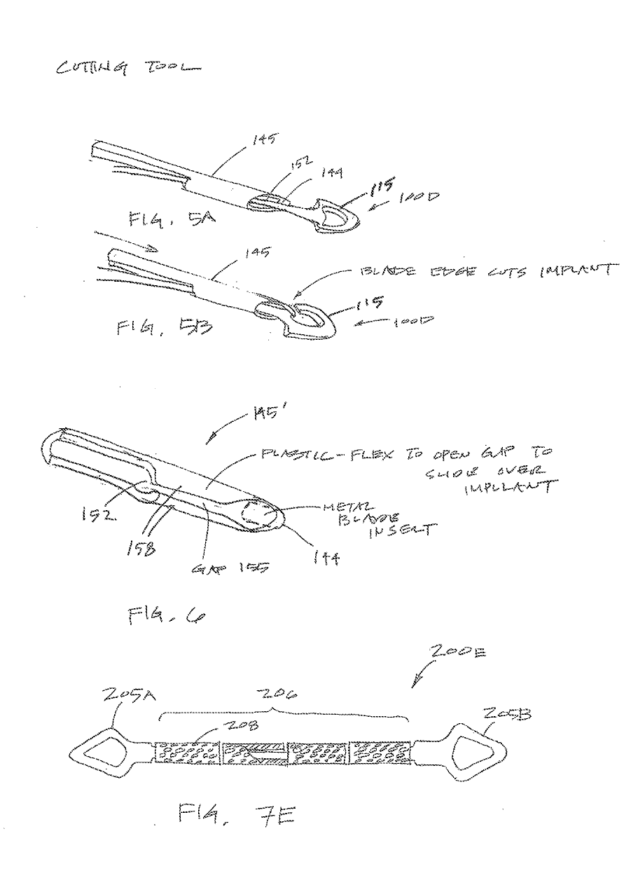

FIG. 4 depicts another embodiment 100D of a revisable implant that is similar to previous embodiments except that at least one end portion 105B includes an indent feature 140 in the proximal-facing aspect of the encircling portion 115 wherein the indent feature 140 is adapted to direct and receive a cutting blade or edge 144 (phantom view) of a cutting tool for cutting the encircling portion of the implant body to allow its removal from the treatment site. As will be described below (FIG. 5B), a cutting tool 145 can be advanced along the medial portion 110 of the implant to sever the end portion, which then will allow the entire implant to be withdrawn from the implant site. In another aspect of the invention, the indent feature 140 in the encircling portion 115 can direct the cutting edge 144 to a reduced cross section portion 148 that will require limited force to cut the polymer element with the cutting edge 144.

FIGS. 5A and 5B illustrate a second component of the kit of a revisable OSA implant system wherein the tool 145 comprises an elongate member with a distal cutting edge 144. One tool embodiment has a passageway 152 extending therethrough for receiving the elongate implant body 100D. In using this tool 145, a first end of the implant body would be freed from tissue or cut and then threaded through the passageway 152. Thereafter, as depicted in FIG. 5B, the tool 145 can be advanced distally while holding the proximal end of the implant to cause the cutting edge 144 to cut across the encircling portion 115. In FIG. 5B, it can be understood how the indent feature 140 and reduced cross section portion 148 (see FIG. 4) direct the cutting edge 144 to easily cut the element to thus release the implant from encircling the tissue plug 112 (cf. FIG. 3B). The tool 145 can be a rigid or semi-rigid member such as a hypotube with a sharpened end. The tool also can be a deflectable, articulatable or deflectable member as in known in the art. In another embodiment, the tool can be a flexible plastic material with a blade insert to provide the cutting edge 144. Referring to FIGS. 5B and 3B, it can be understood that the cut end is flexible and can be pulled from around the tissue plug to extract the implant from the site 120 (see FIG. 3B).

FIG. 6 illustrates another second tool component of system 90 wherein the tool 145′ again comprises an elongate member with a distal cutting edge 144. In one embodiment, the tool end includes a longitudinal gap 155 along a side of passageway 152 to thus allow the tool to be inserted over medial portion 110 of an implant body to then advance and cut the implant as depicted schematically in FIGS. 5A-5B. The tool end as shown in FIG. 6 can comprise a polymer member with flexible elements 158 on either side of gap 155 to allow the device to be inserted over the implant.

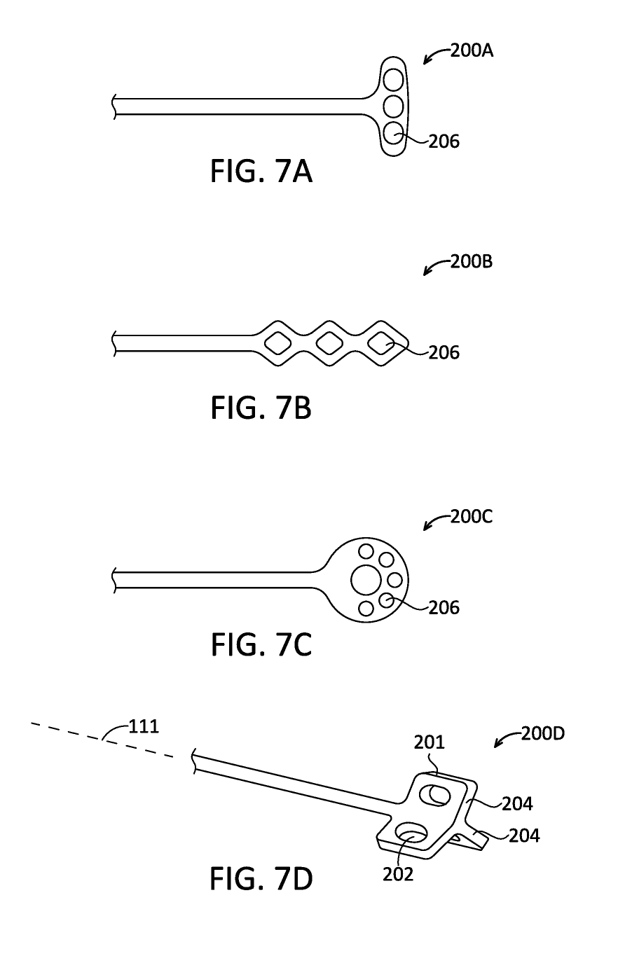

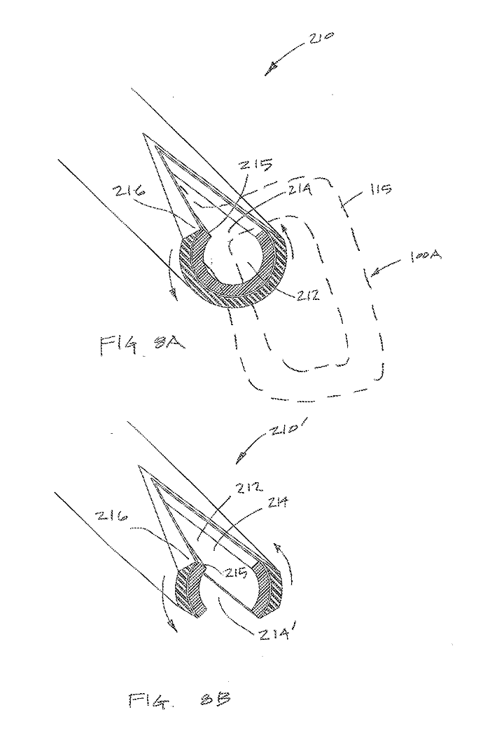

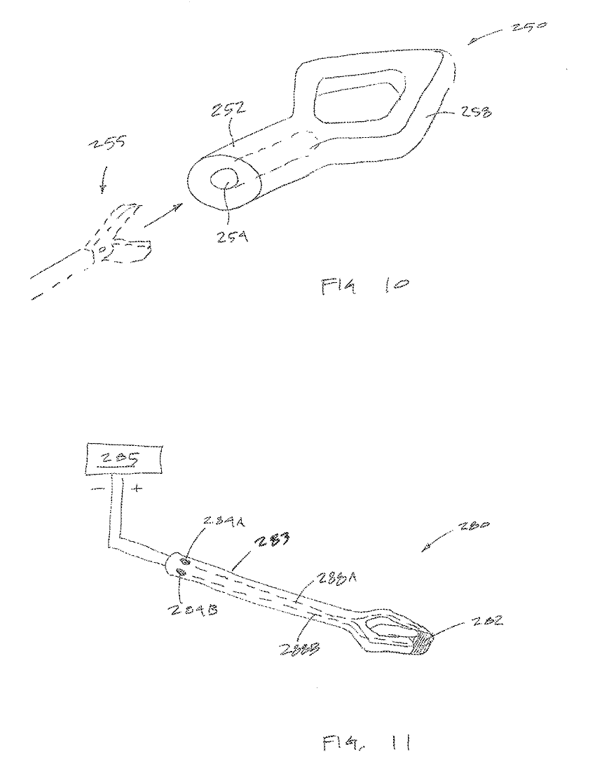

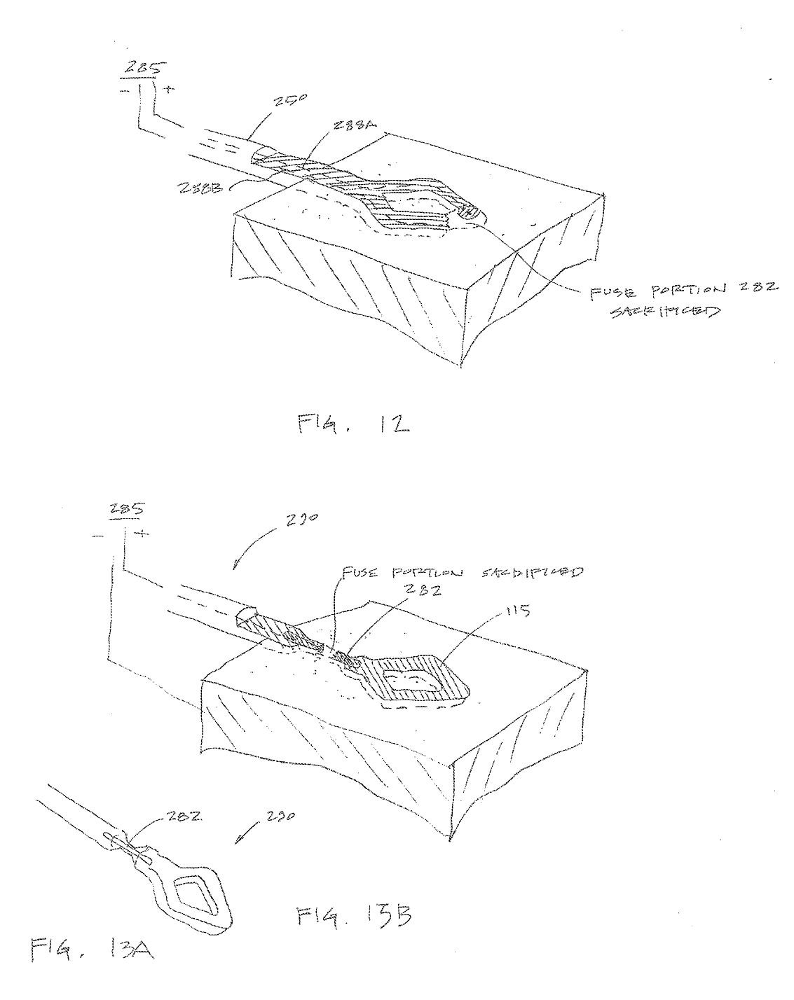

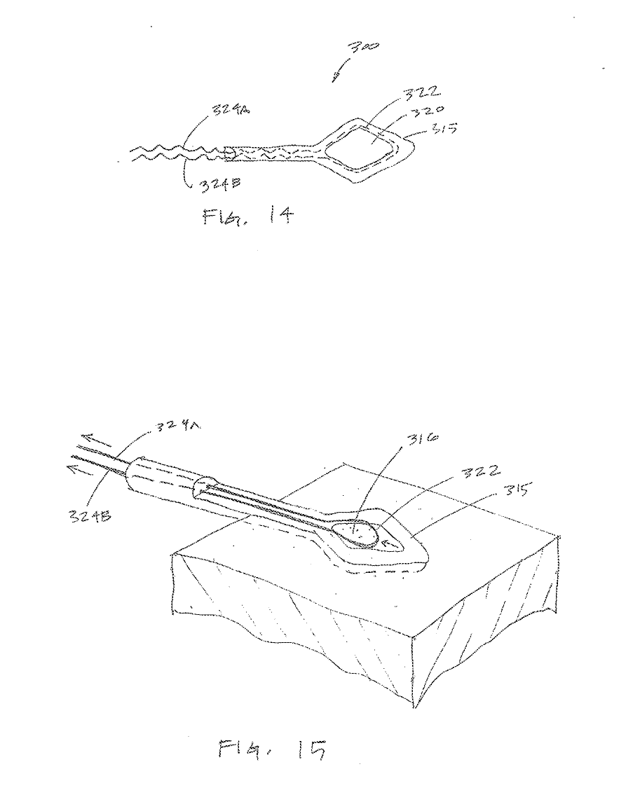

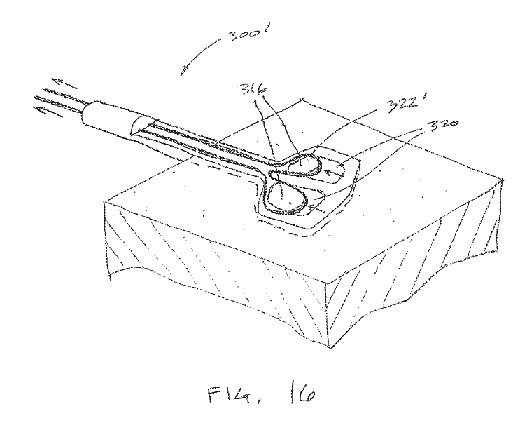

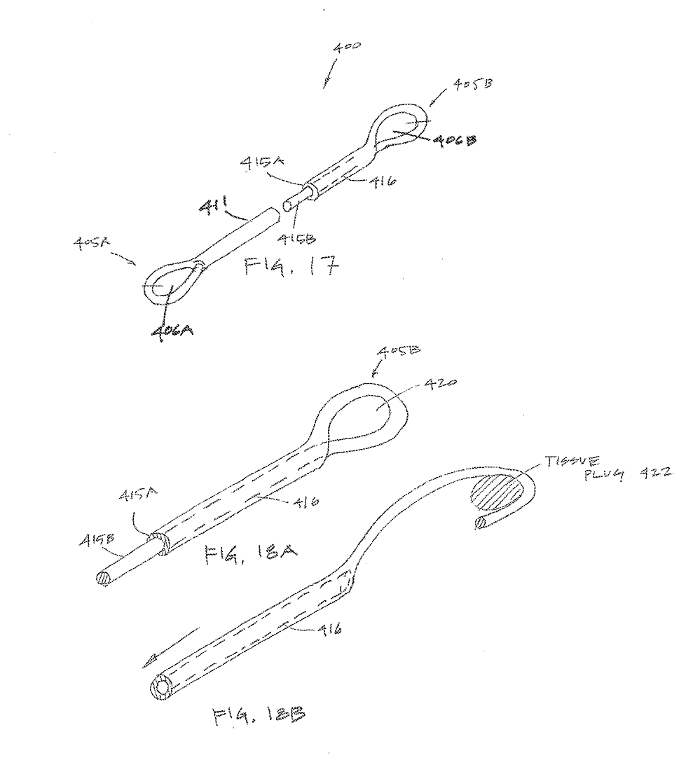

FIGS. 7A-7C illustrate other embodiments of implants 200A, 200B and 200C that each have a plurality of the through-openings 206 in various configurations. In these embodiments, the ends are flat or planar with the openings therein. Thus, in use, there will be a plurality of tissue plugs that grow through the openings to secure the implant ends in the tissue site.

FIG. 7D illustrates another embodiment of implants 200D that has a non-planar end 201 with a plurality of through-openings 202. In one embodiment, the ends have a plurality of elements 204 that extend in different radial angles relative to the axis 111 of the implant with each such element 204 having one or more openings therein.

FIG. 7E illustrates an implant body 200E with ends 205A and 205B and medial portion 206 that comprises an axially-stretched and tensioned elastomeric material. The medial portion 206 is releasably and temporarily maintained in the axially-stretched non-repose condition by a biodissolvable magnesium portion indicated at 208. In this embodiment, the magnesium can comprise a thin wall tube, a plurality of thin wall tube segments, or one or more windings or braids of magnesium. The thin-wall magnesium material, or the magnesium filament of a winding or braid, can be very fine and adapted to dissolve and erode with a selected time interval ranging from about 2 weeks to 52 weeks. In another embodiment, the magnesium portion 208 can be disposed in an interior portion of the implant body, in a linear or helical configuration.

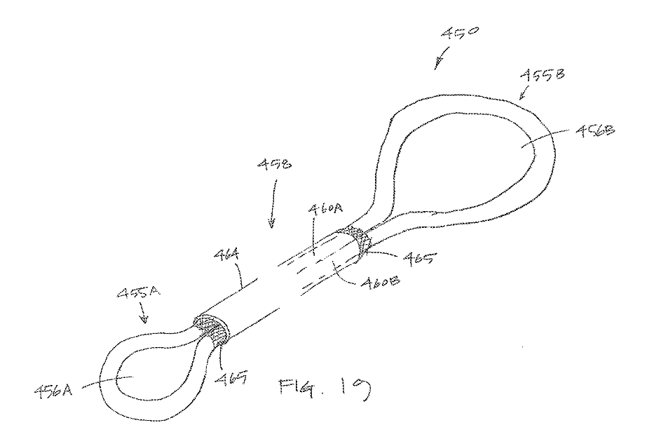

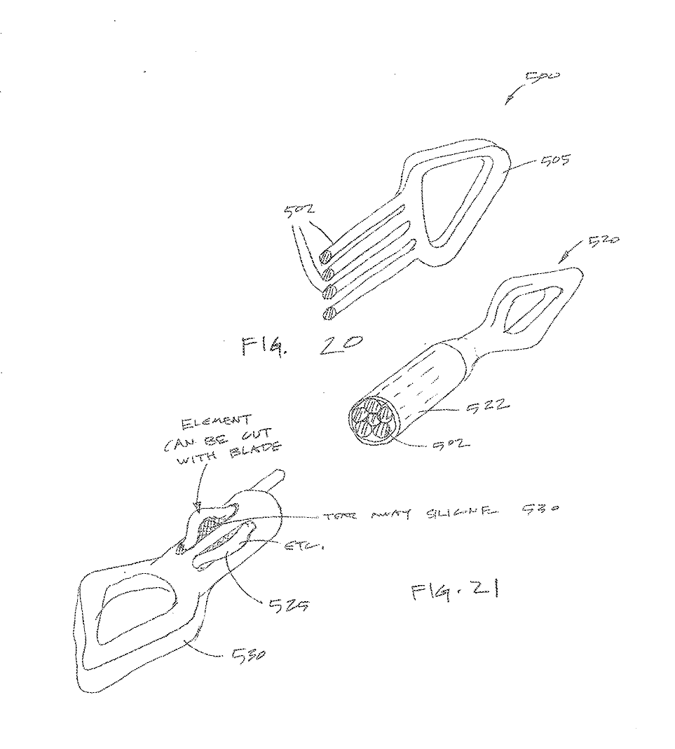

FIG. 8A depicts the working end 210 of an elongated tool that is adapted for cutting an end portion of an implant for its removal, for example an implant of FIGS. 3A-3D, 4, or 7A-7D. The tool functions similar to that of FIGS. 5A and 6, wherein the tool has a central bore 212 that receives the elongate medial portion of an implant body. As can be seen in FIG. 8A, the working end 210 includes two concentric hypotubes with a notch 214 therein to push over an end portion 115 of implant 100A of FIG. 3A, for example. The physician can counter-rotate the hypotubes from a proximal handle end wherein blade edges 215 and 216 of the working end function as a scissors mechanism to cut the implant body. Thereafter, the implant can be easily removed from the treatment site. FIG. 8B illustrates another working end 210′ of a similar cutting tool that has opposing notches 214 and 214′ that can receive a implant body portion and blade edges 215 and 216 can be rotated to cut the implant.

FIG. 9 illustrates another embodiment of implant 220 that is similar to any previous embodiment except depicting a difference in surface characteristics of the implant. In one embodiment, the end or encircling portion 225 can have smooth or slightly textured surface features and the medial portion 230 comprises a highly lubricious surface, and in one embodiment comprises an elastomeric material having an ultrahydrophobic surface 232 to allow for slippage of the tissue against the implant during use. Thus, a method of the invention comprises implanting a device in airway-interface tissue, securing first and second implant end portions in the tissue by permitting a tissue growth through at least one opening in end, and allowing an elastomeric portion of the implant to apply retraction forces to alleviate tissue obstruction of the airway wherein an ultrahydrophobic surface of the implant prevents tissue adhesion to said surface. Ultrahydrophobic surfaces can be provided in a biocompatible polymer, as is known in the art.

In another aspect of the invention, referring to FIG. 9, the elongate implant body is configured for implanting in an airway-interface and at least a portion of a body surface has a wetting contact angle greater than 70°, to prevent tissue adhesion and to allow tissue slippage. In another embodiment, at least a portion of a body surface has a wetting contact angle greater than 85°, or greater than 100°.

In another aspect of the invention, still referring to FIG. 9, the elongate implant body is configured for implanting in an airway-interface and at least a portion of a body surface has an adhesive energy of less than 100 dynes/cm, less than 75 dynes/cm or less than 50 dynes/cm.

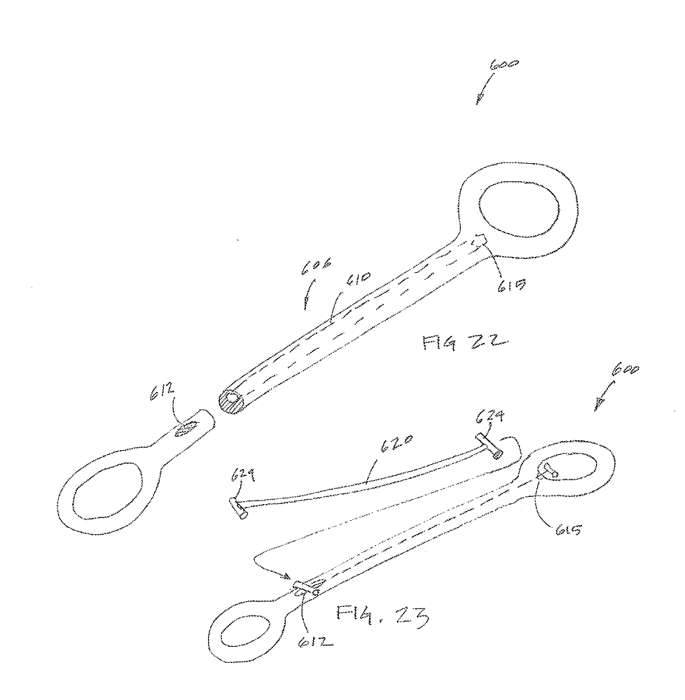

FIG. 10 illustrates another embodiment of revisable OSA implant 250 similar to previous embodiments except the medial portion 252 includes a passageway 254 configured for extending a cutting tool 255 through the passageway for cutting a distal end portion 258 of the implant. The passageway 254 can be accessed by an access opening in the opposing end (not shown) that can be identified by imaging of a marker, visual observation of a marker, by a left-in place guidewire or other suitable means or mechanism. The cutting tool 255 can comprise a scissor member, an extendable blade that is extendable from a blunt-tipped tool, any distal or proximally-facing blade, and/or any type of thermal energy emitter adapted for cutting the implant end 258.

FIG. 11 illustrates another embodiment of revisable OSA implant 280 that has a sacrificial portion indicated at 282 that can be severed or sacrificed by an external stimulus. In one embodiment, a medial portion 283 of the implant includes electrical contacts or extending leads 284A and 284B that can be detachably coupled to an electrical source 285. In FIG. 11, the implant body comprises an elastomeric material as described above and the sacrificial portion 282 comprises a conductively doped polymer portion that acts as a fuse when subject to a very short burst of high voltage RF current. Opposing sides or aspects of the sacrificial portion 282 are coupled to electrical leads 288A and 288B that are embedded or molded into the implant. The use of such doped polymers for a fuse-effect for detachment of endovascular medical implants is disclosed in U.S. Pat. No. 6,458,127 to Truckai et al. and issued Oct. 1, 2002, which is incorporated herein by reference. Similar doped polymers can be used in the revisable OSA implant of FIG. 11.

FIG. 12 illustrates a method of using the OSA implant 280 of FIG. 11, and more particularly for revising the treatment. FIG. 12 depicts that an RF current from source 285 has been delivered to melt, sever and sacrifice portion 282 of the implant thus allowing extraction of the implant from around the tissue plug.

FIGS. 13A and 13B illustrate another embodiment of revisable OSA implant 290 that has a sacrificial portion indicated at 282 in a medial portion of the implant that can be actuated and sacrificed by the external stimulus which then leaves the encircling portion 115 of the implant in place. The left-in-place portion of the implant can be used as an anchor for subsequent implants. In one embodiment as in FIGS. 13A-13B, the sacrificial portion 282 can comprise an electrolytic wire that can be sacrificed over a short time interval by direct current as is known in the art. Such electrolytic wire for detachment of embolic coil implants are known in the field of aneurysm implants and treatments.

While FIGS. 11-13B show OSA implants with two forms of sacrificial portions, it should be appreciated that similar implants can have sacrificial portion that are cut, severed or sacrificed by any external stimulus such as RF current, DC current, light energy, inductive heating etc. and fall within the scope of aspects of the invention.

FIGS. 14 and 15 illustrate another embodiment of revisable OSA implant 300 that again includes at least one end with an encircling portion indicated at 315 that encircles a tissue plug 316 that grows through an opening 320. In one embodiment, the implant carries a cut wire 322 that extends in a loop with first and second wire ends 324A and 324B extending through one or more passageways in the implant. The cut wire 322 can be embedded in the surface of the implant surrounding the opening 320. As can be seen in FIG. 15, the looped cut wire 322 can be pulled proximally to cut the tissue plug 316 which then will free the implant from its attachment. In FIG. 14, it can be seen that the cut wire ends 324A and 324B can have a serpentine configuration in the medial portion of the implant so as to not interfere with the tensioning and relaxation of the elastomeric medial implant portion during its use. When the cut wire is accessed and pulled relative to the implant 300, the tissue plug 316 can be cut. It should be appreciated that other tools (not shown) may be used to stabilize the implant when actuating the cut wire as in FIG. 15. The cut wire 322 can be any form of fine wire, or abrasive wire or a resistively heated wire coupled to an electrical source (not shown).

FIG. 16 depicts another revisable OSA implant 300′ that is similar to that of FIGS. 14-15 with the cut wire 322′ configured to cut a plurality of tissue plugs 316 that have grown through openings 320 within an encircling end portion of the implant body.

FIG. 17 depicts another OSA implant 400 that is adapted for revision as previous implants and system wherein the elongate device or implant body has first and second end portions 405A and 405B with through-openings 406A and 406B therein. The medial portion 411 of implant body 400 extends about an axis and comprises a biocompatible elastomeric material such as a silicone. In this embodiment, the medial portion comprises first and second extending portions 415A and 415B wherein one such portion can be nested in a passageway 416 of the other portion and then form proximal and distal loops or encircling end portions that define openings 406A and 406B for receiving tissue plugs therein. As can be understood from FIGS. 17 and 18A, both the extending portions 415A and 415B comprise an elastomeric material and thus combine to provide the desired retraction forces of the OSA implant.

Referring to FIGS. 18A and 18B, it can be seen that if the second extending portion 415B is cut in a medial or proximal aspect of the implant, or if both the first and second extending portions 415A and 415B are cut in a proximal or medial aspect, then a proximal aspect of the first or outer extending portion 415A can be pulled in the proximal direction and the cut second extending portion 415B then will snake out of the path around the tissue plug 422. Thus, the implant can be cut in a proximal or medial aspect and can be withdrawn from the treatment site from a remote access location.

FIG. 19 depicts another OSA implant 450 that is adapted for a revision procedure and comprises an elongate implant body with first and second end portions 455A and 455B with through-openings 456A and 456B therein. This embodiment is similar to that of FIG. 17 in that medial portion 458 includes extending portions 460A and 460B comprise an elastomeric material that combine to provide the desired retraction forces of the OSA implant. The extending portions 460A and 460B are carried in a thin elastomeric sleeve 464 that has tear-away portions 465 about its ends to prevent tissue ingrowth into the passageway in the sleeve. It can be understood that by cutting the medial portion of the implant, and then pulling on an end of an extending portions 460A or 460B will cause the other free end of the implant to snake around the tissue plug similar to the method depicted in FIG. 18B. Both ends of the implant can be removed from the treatment site by this method.

FIG. 20 depicts another revisable OSA implant 500 that is adapted for minimally invasive in-situ post-implant adjustment of retraction forces applied by the implant. In this embodiment, the implant is configured for a downward adjustment of retraction forces applied by the OSA implant. In FIG. 20, it can be seen that the elongate implant body has a plurality of extending elements 502 coupled to end portion 505, wherein the elements 502 can be individually cut to reduce the applied retraction forces of the implant. The number of extending elements 502 can range from 2 to 20 or more.

FIG. 21 depicts a revisable OSA implant 520 that functions as the previous embodiment except that the plurality of extending elements 502 are housed in thin-wall elastomeric sleeve 522. Further, an axial portion 525 of each extension element 502 protrudes outward from sleeve 522, or an end portion 530 of the implant, to allow such a portion to be cut. Again, any form of cutting tool can be used for minimally invasive access to cut an elastomeric element to titrate retraction forces in a downward direction.

### C. In-Situ Adjustable Force OSA Implants

Another type of OSA implant includes means for in-situ adjustment of force applied by the implant after implantation in the treatment site. Such an adjustment can increase or decrease the applied forces applied to the treatment site by the implant. Such adjustment of forces applied by the implant typically may be performed upon specific event, such as periodic evaluations of the treatment. The adjustment also can be done at a pre-determined schedule, based on an algorithm, or can be random. In one example, the patient may gain or lose weight which could result in a need for adjusting the forces applied by the implant. Other influences can be a worsening of the patient's condition, the aging of the patient, local tissue remodeling around the implant, age of the implant or degradation of material properties of the implant. In another embodiment described below, an implant system can be provided that is easily adjustable in-situ between first and second conditions on a repetitive basis, for example, that can be adjusted for sleep interval and for awake intervals on a daily basis. Such an adjustable embodiment can thus deliver tissue-retraction forces only when needed during sleep. One advantage of such an embodiment would be to allow the tissue of the treatment site to be free from implant-generated retraction forces during awake intervals to prevent or greatly limit the potential of tissue remodeling due to a continuous application of such retraction force. FIG. 22 depicts an OSA implant 600 that is adapted for in-situ post-implant adjustment of retraction forces applied to targeted tissue. In one method, assume that it is desirable to increase the applied retraction forces over time due to tissue remodeling wherein greater retraction forces are desired. In FIG. 22, the elongated implant body has a medial portion 606 that includes an interior channel 610 that extends from an accessible first end 612 to a remote end 615. Each end 612 and 615 can include a silicone membrane to prevent tissue ingrowth but will allow a needle to be inserted therethrough. The channel ends 612 and 615 can be disposed in more rigid end portions of the implant, wherein the medial portion of the implant body comprises an elastomer to provide the desired retraction forces. In one embodiment, the channel 610 is dimensioned to collapse or flatten but can also accommodate the insertion of at least one additional elastomeric element indicated at 620. It can be understood from FIG. 23 that an elastomeric element 620 with end-toggles 624 be inserted in a bore of a flexible needle member (not shown) and inserted through the channel in the implant so that the toggles are released to deploy the element 620 in a tensioned position to thereby add to the retraction forces applied to tissue collectively with the medial portion 606 of the implant 600. In a similar manner, an end of the implant can be clipped to reduce the applied retraction forces as in the system and method depicted in FIGS. 20 and 21.

Thus, in general, the system and implants of FIGS. 20-23 corresponding to aspects of the invention comprise an elongate implant sized and shaped to conform to an airway-interface tissue site in a manner compatible with normal physiological function of the site, a medial portion of the implant comprising an elastomeric material configured to apply retraction forces to the site, and adjustment means for in situ adjustment of retraction forces applied by the implant.

### D. OSA Implants for Applying Non-Aligned Displacement Forces

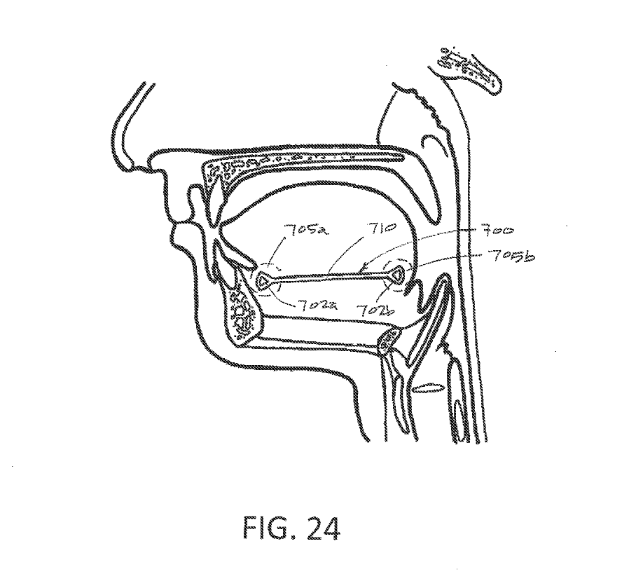

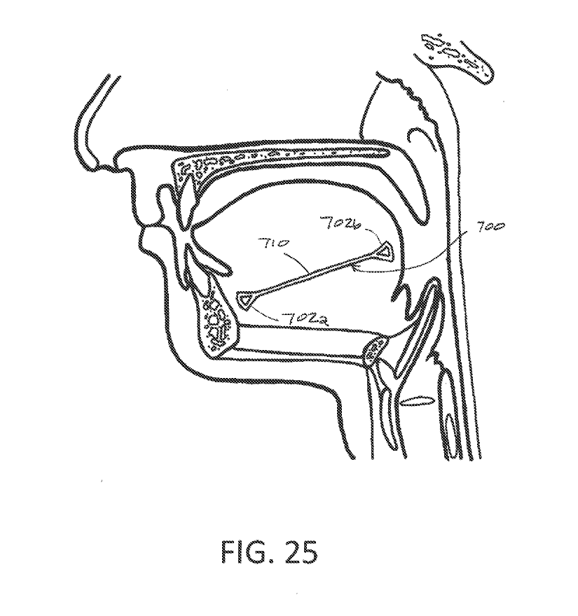

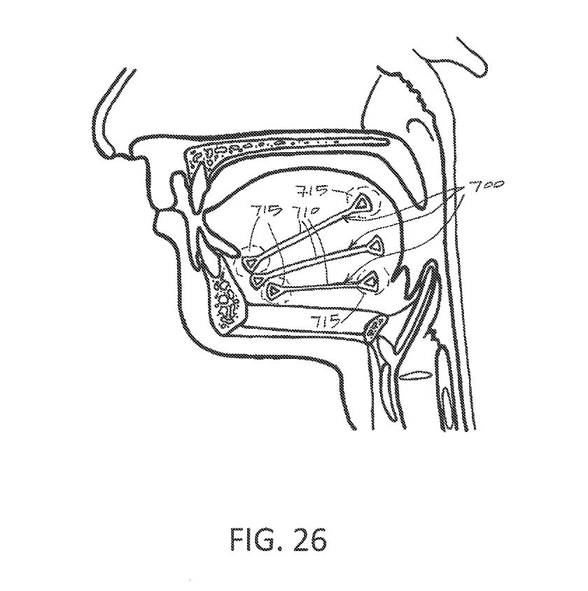

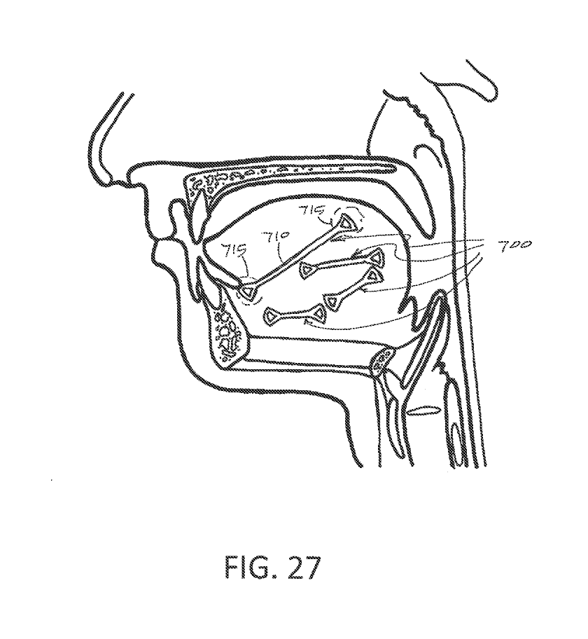

Another aspect of the invention can be described with reference to FIG. 24-27, wherein a resilient implant (or implants) can be positioned in airway-interface tissue to apply tensile forces or displacement forces in at least two non-aligned directions or vectors. In a typical embodiment depicted in FIGS. 24-25, an implant 700 corresponding to aspects of the invention can form a linear structure wherein two anchor ends 702a and 702b form anchor points or regions 705a and 705b in the tissue. Such points 705a and 705b are connected by a straight or substantially straight elastic portion 710 or spring element of the implant such that said elastic portion or spring element applies a tensile force and/or a tensile displacement between said anchor points 705a and 705b. In the embodiment of FIG. 24, the implant 700 acts to apply forces and/or displacements between the said anchor points 705a and 705b to displace and/or apply forces to the patient's tongue, but it should be appreciated that an appropriately dimensioned implant can also or instead be introduced into the soft palate or pharyngeal structures adjacent to the patient's airway. FIG. 25 illustrates the implant 700 can have various orientations in the tissue. Now turning to FIGS. 26-27, it can be seen that a plurality of substantially linear elastic implants 700 similar to that of FIGS. 24-25 can thus provide a plurality of tissue anchor points 715 wherein the elastic or spring portion 710 of the implants function in such a manner to provide tensile or displacement forces to achieve the desired clinical effects. Testing in animal models has indicated that forces applied to the subject's tongue by two implants in two different directions may improve implant performance when compared with unidirectional application of forces from a single implant.

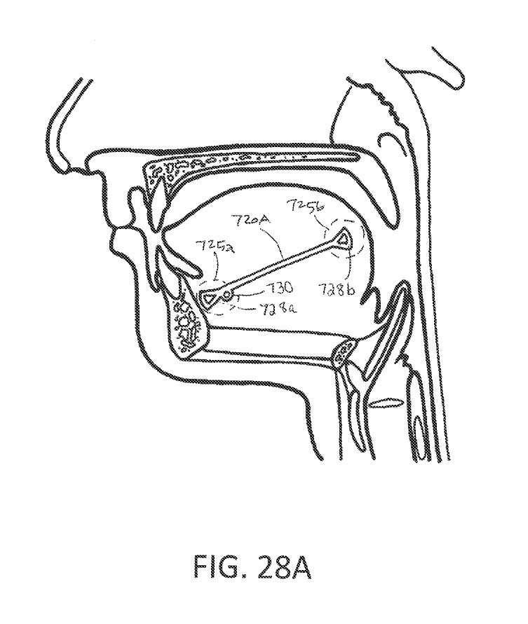

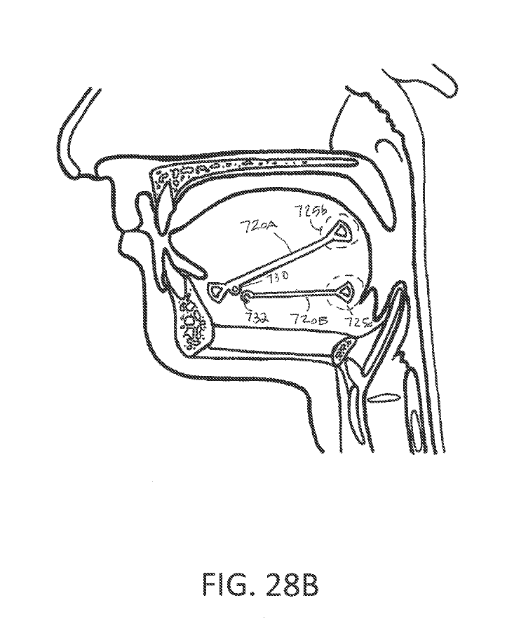

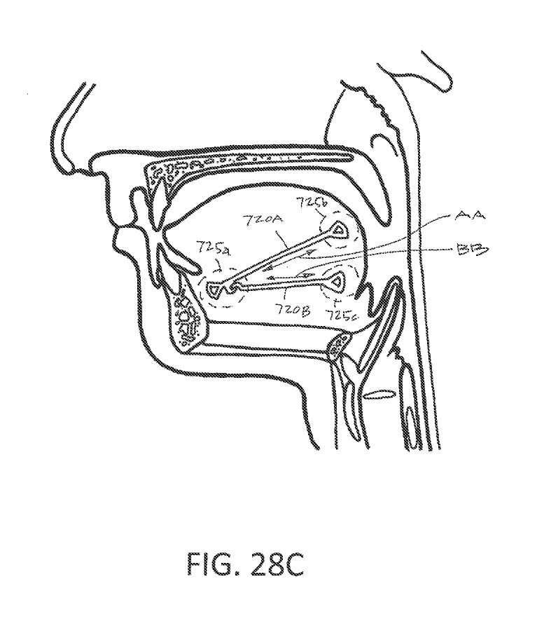

FIGS. 28A-28C schematically illustrate another embodiment of implant system according to aspects of the invention that comprises first and second elastic elements 720A and 720B that provide three anchor points in tissue indicated at 725a, 725b and 725c. FIG. 28A depicts the implantation of the first elastic element 720A which has anchoring ends 728a and 728b as described above, wherein at least one end is configured with an attachment element such as a loop 730 that is connectable with a hook element 732 of a second elastic element 720B. Thus, FIGS. 28A and 28B depict the steps of implanting the elastic elements wherein elastic element 720A is initially implanted in its desired location. Then, FIG. 28B depicts elastic element 720B being positioned in its desired location such that the hook 732 is adjacent to loop 730 of the elastic element 720A. FIG. 28C then depicts the loop 730 and hook 732 be connected in such a manner to produce a fixed-link implant structure which thus applies forces in two non-aligned vectors AA and BB. It can be understood that the implants can be implanted in sequence and then coupled in situ to form a V-shaped implant system. It should be appreciated that the implant structure of FIGS. 28A-28C can have components such as elastic or spring elements that can be connected prior to, during, or following implantation by means of adhesives, connectors, snap-fit features, hooks and loops, clamps, ratchets, keyed fittings, etc., or by means of separate attachment, such as sutures, junctions, clamps, or other connection means. In another embodiment, two end portions of separate implant bodies can be disposed proximate to one another, and the body's fibrotic response or wound healing response can cause a connection of the two implant ends.

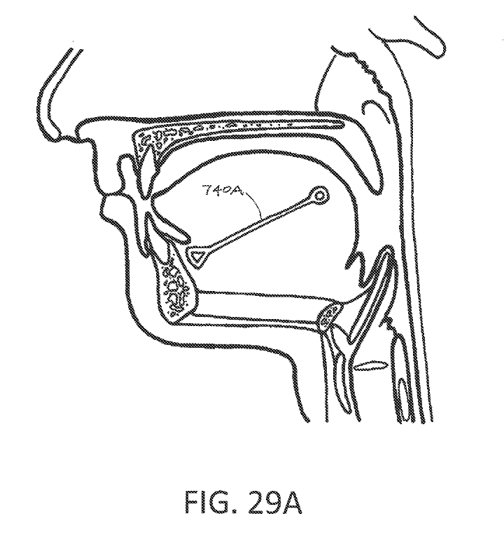

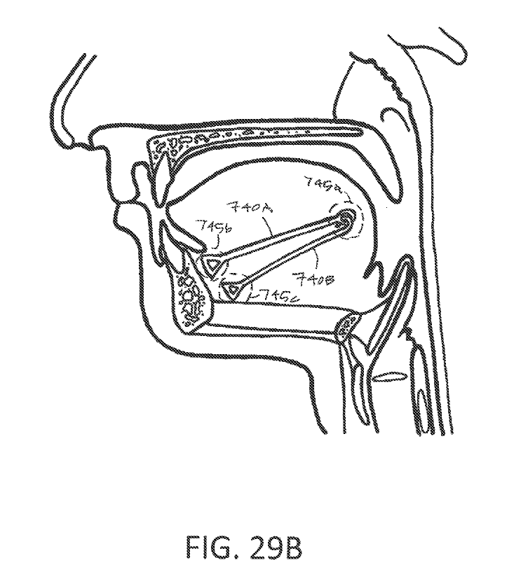

FIGS. 29A-29B schematically illustrate another embodiment of implant system comprising first and second elastic elements 740A and 740B in a different orientation in a patient's tongue. Each implant has an elastic medial section as described above. The implant system again provides three anchor points 745a-745c as shown in FIG. 29B, wherein the first implant can be fixedly attached to the second implant by loop and hook features or other similar means. As described previously, the implants can be implanted in sequence and then coupled in situ to form the V-shaped implant system. In some embodiments, the angle between the legs of V-shaped implant can range from about 10° to 170°, depending on the implant site. The lengths of the legs of the V-shaped implant can vary, as well as the forces applied by each leg of the V-shaped implant.

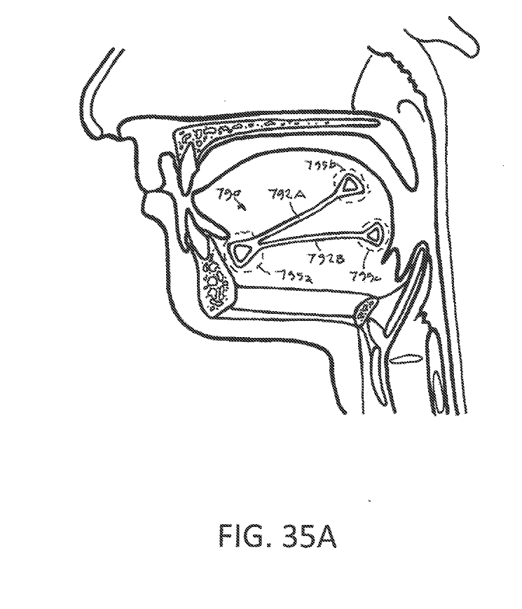

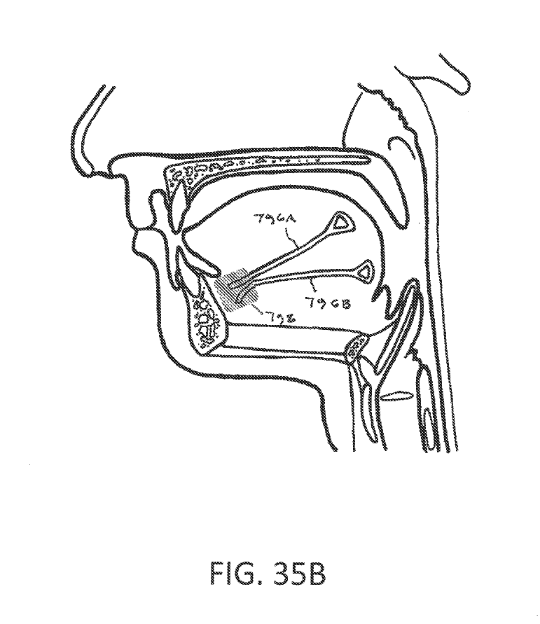

In general, when the implants of the disclosure as described above are implanted in the tongue and/or the palate of the patient (FIG. 35), the positioning of the implants will affect the location and direction of the applied forces and the displacements of the surrounding tissues. The implants may be placed in various locations to achieve the desired clinical effects, and may be specifically tailored to an individual patient based on the nature and details of each patient's OSA, including their specific anatomy and physiology. For example, if a patient suffers obstructions associated with the lower posterior region of the tongue impinging on the posterior pharyngeal wall, then an implantation location that places one end of a linear implant lower in the tongue may be appropriate (see FIG. 24). In another example, if the patient suffers obstructions associated with the upper posterior region of the tongue impinging on the posterior pharyngeal wall, then an implantation location that places one end of a linear implant higher in the tongue may be more appropriate (see FIG. 25). In a similar manner, the implants of the disclosure may be placed in various locations within the tongue and soft palate, utilizing one or more implants, to address the specific needs of the patient and to achieve the desired clinical effects.

In general, a method according to aspects of the invention for treating an airway disorder comprises implanting at least one elastic implant in airway-interface tissue wherein the at least one implant in configured to apply tensile forces to the tissue in at least two non-aligned directions or vectors. The non-aligned vectors thus describe the linearly-directed forces applied to tissue by substantially linear, elongated implants disposed in the tissue, such as vectors AA and BB in FIG. 28C.

In one aspect of the method, the linearly-directed forces can be applied to tissue in the non-aligned vectors by a single implant configured with first and second body portions that extend in between different anchoring sites (see FIG. 35). In another aspect of the method, at least first and second implants can be implanted to apply such forces in at least first and second non-aligned vectors. In any implant embodiment, the elongated elastic body portions can cooperate with bioerodible materials that temporarily maintain the implant in an extended position as described above. Further, as described previously, the targeted airway-interface tissue which receives the implant can comprise the patient's tongue, soft palate and/or pharyngeal tissue.

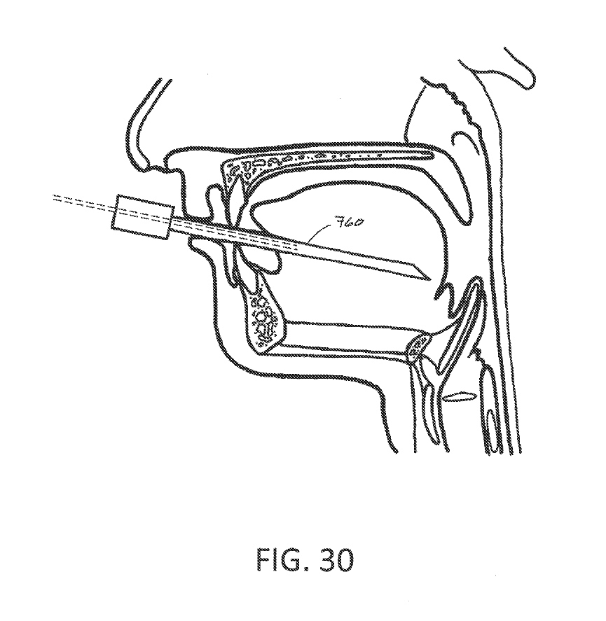

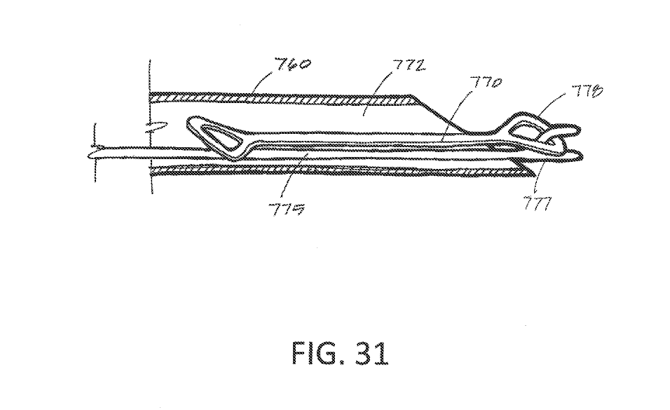

Now turning to FIGS. 30-34, various aspects of the invention are described that relate to placement of the implants within the tongue or soft palate of the patient. Implantation may be achieved in a variety of manners, and typically is accomplished by the insertion of a needle-based cannula 760 as shown schematically in FIG. 30. It should be appreciated that an open surgery or other minimally invasive surgical technique can be used. In one embodiment of sharp-tipped cannula 760 shown in FIG. 31, the implant body 770 is carried in bore 772 of the cannula. A thin push rod or stylet member 775 has a distal end 777 that releasably engages a distal portion 778 of the implant body. The engagement can comprise a hook or other attachment means for coupling with the distal end of the implant body. The stylet 775 can reside in the cannula bore 772 alongside the flexible implant body in such a manner that when said stylet is pushed, the distal end of the stylet functions pull or deploy the implant 770 through said cannula, avoiding any jamming or bunching of said implant during deployment. Further, the implant can be deployed in the targeted tissue site in a fully elongated (i.e. non-bunched) fashion. In another aspect of the method, the cannula is introduced into the targeted site, and thereafter the physician maintains the stylet 775 in a fixed position and contemporaneously withdraws the cannula 760 to thus deploy the implant body 770 in the targeted site.

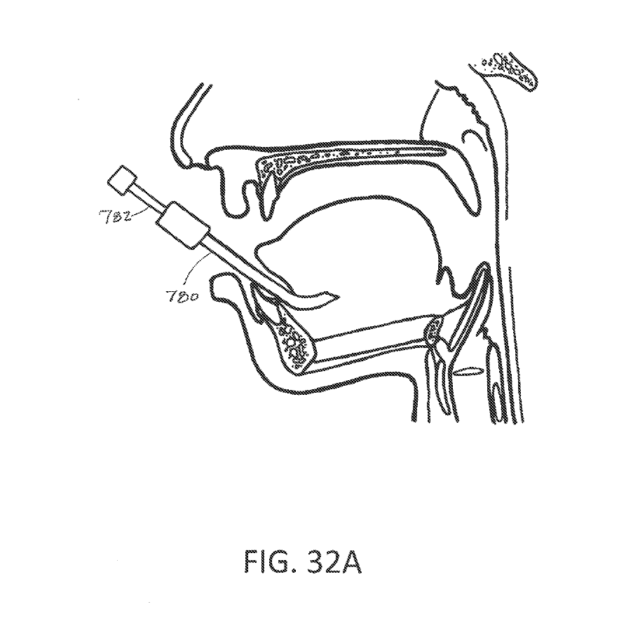

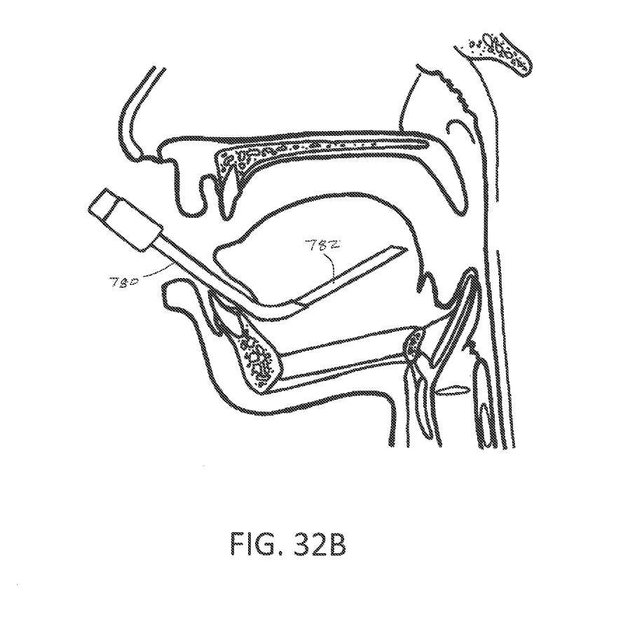

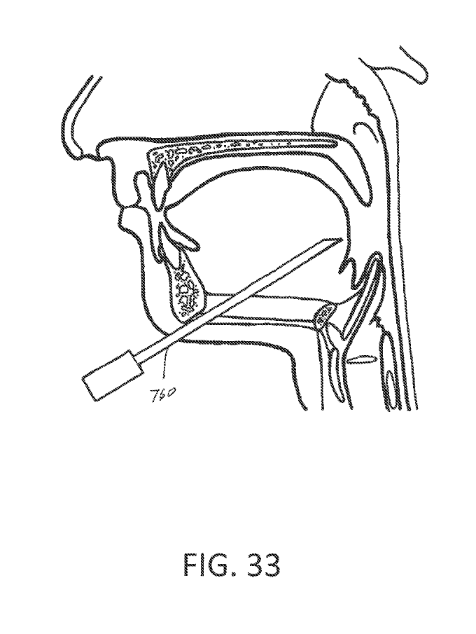

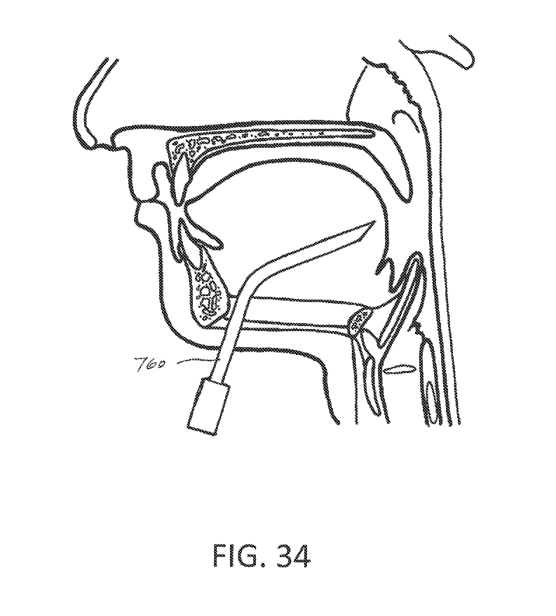

The disclosed implants may be placed within the tongue by means of straight, curved, articulating, deformable or telescoping cannulas 760 as in FIGS. 30-34, which may be introduced through any access points described above. The route of access to the implantation site within the tongue may include access via a sublingual location as depicted in FIGS. 30 and 32A-32B, (within the oral cavity, below the anterior portion of the tongue), access via a submandibular location as depicted in FIGS. 33-34 (below the anterior portion of the mandible), access via a posterior lingual location (on the posterior surface of the tongue) or any other access point that may allow for proper implant positioning.

The route of access to the implantation site within the soft palate may include access via an intra-oral location (within the oral cavity adjacent to the junction of the soft palate and the hard palate) or an intra-nasal location (within the nasal cavity adjacent to the junction of the soft palate and the hard palate), or any other access point along the soft or hard palate that may allow for proper implant positioning.

In one example, FIG. 30 shows a straight cannula inserted in the sublingual location, resulting in a substantially straight placement with the anterior anchor located adjacent to a superior part of the mandible. In another example, FIGS. 32A-32B depict an angled, bendable, or articulating cannula 780 with a telescoping secondary cannula 782 inserted in the sublingual location which would result in a substantially straight implant placed with the anterior anchor portion of the implant located adjacent to a superior part of the mandible.

FIG. 33 depicts a straight cannula 760 inserted in the submandibular location which would result in a substantially straight implant placement with the anterior anchor located adjacent to an inferior part of the mandible. In another example, FIG. 34 shows a curved cannula inserted from a submandibular location which results in a slightly curved position with the anterior anchor located adjacent to a mid-level position on the mandible.

In another embodiment, the second sleeve may have memory shape (e.g. NiTi) or may be a plastic sleeve.

The disclosed implants as described above are substantially flexible, and are typically fabricated of flexible and/or elastic materials such as silicone, urethane, fluoroelastomer, or other bio-compatible elastomers, polyethylene terephthalate (e.g. Dacron®) or other fibers, bioabsorbable polymers, flexible metals or the like. The flexibility of the implants allows for such implants to be easily deployed and implanted through small cross-section cannulas, which may be straight, curved or articulated, without the implant body jamming within the cannula bore. Longer implants may be delivered through curved or bent cannulas than would be possible with stiff or rigid implant materials or designs.

Because such implants are substantially flexible, pulling the implants, instead of pushing them, through the cannulas may be advantageous for certain applications, such as narrow, straight, curved, deformable or articulated cannulas. The primary advantage of pulling or deploying a flexible implant from such a curved or straight cannula is an increased resistance to bunching, buckling, or otherwise jamming in the cannula bore. This aspect of the deployment method allows such flexible implants to be delivered around tight bends in the cannula, thus enabling implantation in difficult to reach locations such as delivery within the tongue through the sublingual space (see FIGS. 31-32B). Pulling also allows longer implants to be delivered than would otherwise be the case. In another embodiment, only the end portions of the implant are deformable.

FIG. 35A schematically illustrates another embodiment of implant 790 that comprises a unitary implant body with first and second elastic elements (“legs”) 792A and 792B that may be deployed in different orientations in a different patients' tongues. It can be understood that implant 790 of FIG. 35A can be implanted by means of a primary cannula carrying two resilient curved stylets (or secondary slotted cannulas, not shown) that are deployed from the primary cannula. The implant 790 again provides three anchor points 795a-795c as shown in FIG. 35. As described above, the V-shaped implant 790 can have any suitable angle between the legs 792A and 792B and any suitable forces can be applied by each leg of the V-shaped implant.

FIG. 35B depicts first and second OSA implants 796A and 796B that are introduced with at least a portion of the implants in close proximity. Thereafter, a fibrotic response indicated at 798 may be induced that can effectively couple the ends of the implants to again provide a V-type implant wherein the first and second implants apply linear-directed forces in different vectors.

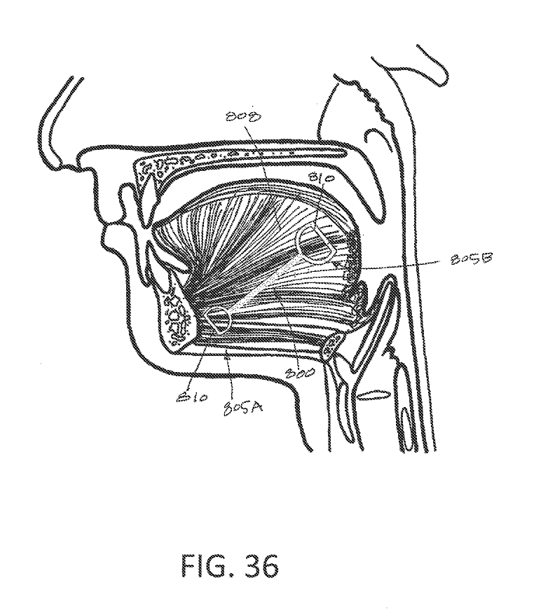

Exemplary implants of the disclosure can be configured with anchor portions at various locations along the implants, including the ends, or distributed along the length of the elastic or spring elements of the implant, or adjacent to the elastic or spring elements and serve to attach the implants to tissue. The tissue can comprise soft and hard tissues and structures, including skin, mucosa, muscle, fascia, tendon, ligament, cartilage, or bone so as to allow the elastic or spring elements to apply forces and/or displacements to said soft tissue, hard tissues or structures. When employed within a patient's tongue, the anchor portions of such implants can form attachments directly within tongue muscles, including the geniohyoid, the genioglossus, the vertical, the transverse, and the longitudinal muscles. The geniohyoid, the genioglossus, and the vertical muscles within the tongue substantially run in a direction from their attachments at the central anterior portion of the mandible and fan outward isentropically toward the posterior and superior oral cavity surfaces where the transverse and longitudinal muscles reside (FIG. 36). As described above, the anchor portion of the implant can attach by means of tissue plugs through holes in the anchor portions, ingrowth of muscle tissue into channels, passages, pores, or other interstitial spaces in the anchor portion of the implant body.

The implants of the disclosure may be implanted in such a manner and in specific orientations so as to encourage the isentropic muscle tissue to in-grow and attach to said anchors to encourage specific characteristics. These characteristics may include, but are not limited to, accelerated or delayed attachment to said muscle tissues, stronger or weaker attachments, isentropically strengthened attachments, reduced or increased stiffness of the attachments, reduced pain or sensitivity of the attachments.

In another aspect of a method of the invention, an implant 800 (FIG. 36) has end portions or anchoring portions 805A and 805B that are configured with elements, surfaces and surface areas that allow for tissue plugs or tissue growth therein that resist unwanted movement of the implant end within tissue planes, such as along the surface of muscle fibers 808. FIG. 36 depicts the orientation of muscle fibers 808 in a patient's tongue. More in particular, referring to FIG. 36, the implant 800 has end portions 805A and 805B each with an element 810 that is configured to extend transverse a selected dimension to such muscle fibers 808. The length of the feature or element 810 that extends transverse to muscle fibers can be at least 2 mm, 4 mm, 6 mm or 8 mm to thereby provide assurance that the implant will not migrate in an intra-muscle fiber tissue plane.

In another aspect of the invention one or more of the anchoring portion can be a composite structure (e.g. a polyester fiber reinforced silicone rubber or a substantially non-elastic polymer or metal). The composite structure may limit loss of applied force that might otherwise occur due to stretching of the anchoring portion.

In another aspect of the method of invention, referring to FIG. 36, the implant body 800 is positioned in a targeted site, such as a patient's tongue, such that the forces applied by the elastic portion of the implant are substantially aligned with the direction of contraction (or axis) of contractile muscle fibers 808 and wherein the anchoring portions of the implant body 800 include tissue engaging elements that extend substantially transverse to the axis of such contractile muscle fibers 808.

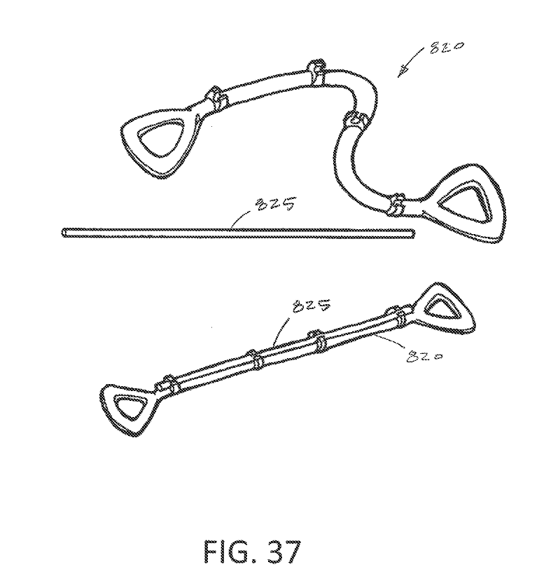

FIG. 37 illustrated another embodiment of flexible implant 820 which can be temporarily maintained in an elongated position. In this embodiment, the implant 820 carries a semi-rigid rod 825 of a bioabsorbable material (e.g., a bioabsorbable polymer) embedded or locked into features on a surface of the implant body. The implant thus can be configured with sufficient buckling strength so that the implant 820 and bioabsorbable rod 825 can be pushed through a cannula that may be straight, bent, curved, or articulated, without jamming or bunching. This embodiment provides an alternative means for implant deployment rather than the stylet deployment of FIG. 31.

E. Implant Force and/or Movement Parameters

Implant Force Threshold.

The implants of the disclosure may apply forces and displacements to anatomical structures within the patient's airway, including the tongue and soft palate, to treat obstructive sleep apnea (OSA) by repositioning and/or applying forces to said anatomical structures in such a manner as to provide an open airway during normal breathing. The forces applied by said implants to said anatomical structures are large enough to sufficiently to move, or displace, said structure so as to provide a clear airway when the patient is asleep, but are not so large as to damage the surrounding tissue, damage the implant, prevent proper airway function, or prevent proper tongue function such as normal speech and swallowing.

When the one or more implants of the disclosure are employed within the patient's tongue to prevent airway occlusion associated with OSA when said patient is asleep and fully relaxed, said implant(s) provide sufficient force to allow the airway to open during normal breathing. The force necessary to open said airway during normal breathing may be a force less than the weight of the tongue itself, as normal breathing provides an internal pressure that acts to help open the airway. The minimum force supplied by said implant(s) to allow the airway to open during normal breathing is referred to as the minimum threshold force for therapeutic benefit. This minimum threshold force for one or more implants within or adjacent to the tongue is 0.5 Newtons in some embodiments, the minimum threshold force is 1.5 Newtons in other embodiments, and the minimum threshold force is 3.5 Newtons in still other embodiments.

When one or more implants of the disclosure are employed within the patient's soft palate to prevent airway occlusion associated with OSA when said patient is asleep and fully relaxed, said implant(s) provide sufficient force to deflect the soft palate away from the back wall of said patient's throat thus providing an open airway. As with the tongue, the force necessary to open said airway during normal breathing may be a force less than the weight of the soft palate itself, as normal breathing provides an internal pressure that acts to help open the airway. The minimum force supplied by said implant(s) to allow the airway to open during normal breathing is referred to as the minimum threshold force for therapeutic benefit. This minimum threshold force for one or a more implants within or adjacent to the soft palate is 0.2 Newtons in some embodiments, the minimum threshold force is 0.5 Newtons in other embodiments, and the minimum threshold force is 1.0 Newtons in still other embodiments.

Implant Motion Threshold.

The implants of the disclosure apply forces and displacements to anatomical structures within the patient's airway, including the tongue and soft palate, to prevent obstructive sleep apnea (OSA) by repositioning said anatomical structures. The displacements applied by said implants to said anatomical structures are large enough to sufficiently move, or displace, said structures so as to provide a clear airway when the patient is asleep, but are not so large as to cause adverse side effects. Said side effects may include limited tongue or soft palate function resulting in adverse effects on speech and/or swallowing, difficulty breathing, unwanted remodeling of tissues over time, damage to soft or hard tissues, and causing said soft structures, like the tongue or soft palate, to interfere with other anatomical structures or to cause other unwanted effects.

When implanted within the tongue, the implants of the disclosure provide forces and displacements to the tongue to allow the patient's airway to remain open during normal breathing when the patient is asleep and fully relaxed. The maximum displacement of the tongue that does not result in undesired side effects, as mentioned above, is referred to as the maximum threshold displacement for therapeutic benefit. This maximum threshold displacement for one or a more implants within or adjacent to the tongue is between about 0.5 mm and about 20 mm in some embodiments, between about 1.0 mm and about 15 mm in other embodiments, and between about 1.0 mm and about 10.0 mm in still other embodiments.

When implanted within the soft palate, the implants of the disclosure may provide forces and displacements to the soft palate to allow the patient's airway to remain open during normal breathing when the patient is asleep and fully relaxed. The maximum displacement of the soft palate that does not result in undesired side effects, as mentioned above, is referred to as the maximum threshold displacement for therapeutic benefit. This maximum threshold displacement for one or a more implants within or adjacent to the soft palate is from 0.5 mm to 5.0 mm.

When implanted in the tongue, the implants of the disclosure may provide an effective therapeutic window of operation bounded by a minimum threshold force required to prevent the tongue from obstructing the airway during normal breathing when the patient is asleep and relaxed, and by a maximum displacement threshold above which the implant(s) adversely affects normal airway and tongue function including speech, swallowing, breathing, etc. This effective therapeutic window is identified based on the forces and displacements described above.

When implanted in the soft palate, the implants of the disclosure may provide an effective therapeutic window of operation bounded by a minimum threshold of force required to prevent the soft palate from obstructing the airway when the patient is asleep and relaxed, and by a maximum displacement threshold above which the implant(s) adversely affects normal airway or mouth function including speech, swallowing, breathing, etc. This effective therapeutic window is identified based on the forces and displacements described above.

Implant Force/Motion Directions within the Tongue.

When the one or more implants of the disclosure are employed within the patient's tongue to prevent airway occlusion when said patient is asleep and fully relaxed, said implant(s) provide sufficient force to open the airway during normal breathing. One or more implants may be employed to apply the desired forces and deflections to the patient's tongue. Said implants may be employed in one or more locations within or adjacent to the tongue, they may be anchored in one or more locations within or adjacent to the tongue, and they may apply forces and/or deflections in one or more directions and between two or more locations within or adjacent to the tongue.

Said implants may be employed in such a manner as to relieve obstructions in the airway caused by the tongue resulting in OSA. Generally, this includes displacing the posterior region of the tongue and/or providing forces on the posterior region of the tongue that pull said posterior region in the anterior direction, away from the posterior pharynx wall, resulting in keeping the opening of the airway the airway from closing such that normal breathing can be maintained. Said forces and/or displacements may act to affect the entire posterior region of the tongue, a very specific location in the posterior region of the tongue, a linear area of affect in the posterior region of the tongue (i.e., a linear area that runs cranially and caudally so as to create a channel through which the airway remains patent), or any combination of the above.

In one example exemplary embodiment, a single implant is employed to apply a force to the posterior region of the tongue in an approximately horizontal anterior direction as viewed in a patient standing straight up with their head facing forward (FIG. 24). In another exemplary embodiment, a single implant is employed to apply a force to the posterior region of the tongue at an inclined angle to the horizontal, and in the anterior direction as viewed in a patient standing straight up with their head facing forward (FIG. 25).

In another embodiment of the invention, three implants are employed within the tongue to apply forces to the posterior region of the tongue in such a manner as to advantageously create a longitudinal open region between said tongue and the posterior pharyngeal wall, running in the direction of air motion during normal breathing. The three implants in this embodiment are acting in different directions to create the desired net distribution of forces and displacements on the tongue (FIG. 26). In another embodiment of the invention, four implants are employed within the tongue to apply forces distributed throughout the tongue, with the implants acting in different directions to create the desired net distribution of forces and displacements on the tongue (FIG. 27).

When more than one implant is used, the set of implants may all lie in any orientation with regard to each other and the surrounding anatomical structures, including in a linear arrangement, a parallel arrangement, a planar array (including but not limited to a triangulated structure), a three-dimensional array, or any combination of these arrangements. The implants may be joined together in any multi-linear, non-linear, or multiply-linearly segmented manner. One example is described above in FIGS. 28A-28C, wherein two linear elastic or spring elements 720A and 720B are connected to provide a common anchor point 725a in tissue at one end of each of the two said linear elements, respectively. The other ends of the first and second linear elements provide additional anchor points 725b and 725c in the tissue. In this manner, anchor points 725b and 725c are pulled in the direction of the common anchor 725a so as to provide a bi-linear implant structure. By extension, and in this manner, complex multi-linear structures or networks of linear elements may be constructed to achieve the desired clinical effects. Similarly, two or more implants comprising multi-linear components may be employed in conjunction to achieve the desired clinical effects. Alternately, the elastic or spring elements may be fabricated in such a fashion as to produce a joined, jointed, or linked structure during the manufacturing process.

Implant Force/Motion Directions within the Soft Palate.

When the one or more implants of the disclosure are employed within the patient's soft palate to prevent airway occlusion when said patient is asleep and fully relaxed, said implant(s) provide sufficient force to open the airway during normal breathing. One or more implants may be employed to apply the desired forces and deflections to the patient's soft palate. Said implants may be employed in one or more locations within or adjacent to the soft palate, they may be anchored in one or more locations within or adjacent to the soft palate, and they may apply forces and/or deflections in one or more directions and between two or more locations within or adjacent to the soft palate.

Said implants may be employed in such a manner as to relieve or prevent obstructions in the airway caused by the soft palate resulting in OSA. Generally, this includes displacing the posterior region of the soft palate and/or providing forces on the posterior region of the soft palate that pull said posterior region in the anterior direction away from the posterior wall of the pharynx resulting in the opening of the airway during normal breathing. More specifically, said implants within said soft palate tend to cause a curvature of the soft palate in the downward and anterior direction to affect said opening of said airway. Said forces and/or displacements may act to affect the entire posterior region of the soft palate, a very specific location in the posterior region of the soft palate, a linear area of affect in the posterior region of the soft palate, or any combination of the above.

In one exemplary embodiment, a single implant is employed to apply a force to the posterior region of the soft palate resulting in a curvature of said soft palate that displaces said soft palate away from the pharynx wall. In another embodiment of the invention, two implants are employed within the soft palate at differing angles and in different locations to apply forces and displacements to the soft palate resulting in a curvature of said soft palate that displaces said soft palate away from the pharynx wall.

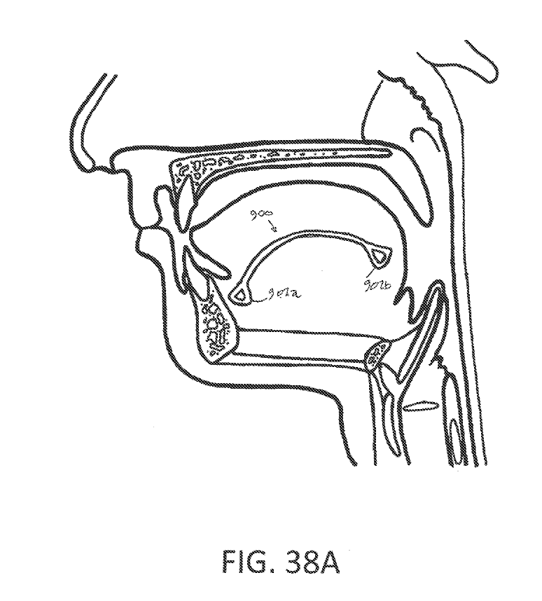

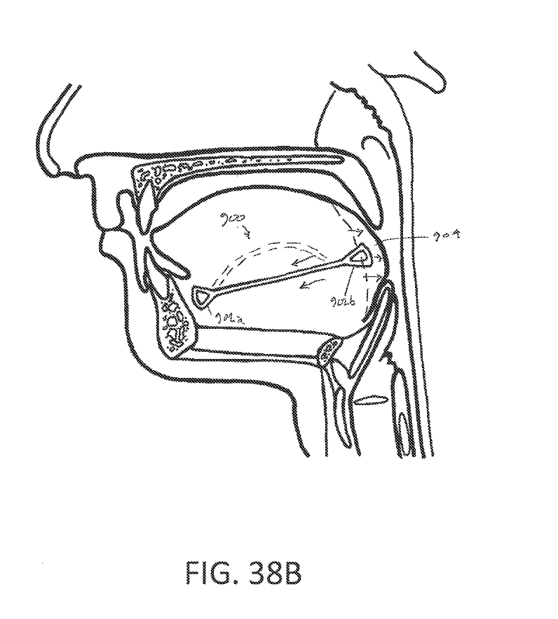

The above-described OSA implants in FIGS. 24-37 generally describe implant bodies and methods that are adapted to apply linearly-directed forces to airway interface tissue. Other embodiments described next relate to implants configured to displace tissue or apply forces in non-linear vectors, which can be used alone or in combination with the linear force-directing implant described previously. In one embodiment, FIGS. 38A-38B depict an elastic OSA implant 900 with anchor ends 902a, 902b that is curved in a repose state and can be implanted in either a curved or linear path, for example, in a vertical orientation in the patient's tongue (FIG. 38A). In FIG. 38B, it can be seen that if tongue base 904 is displaced posteriorly, the implant will be moved toward a straightened configuration wherein the elastic implant will apply forces anteriorly and upward to prevent airway interference. The implant of FIGS. 38A-38B can have any suitable ends for anchoring in tissue, for example, end portions with one or more openings resulting in tissue plugs anchors as described above.

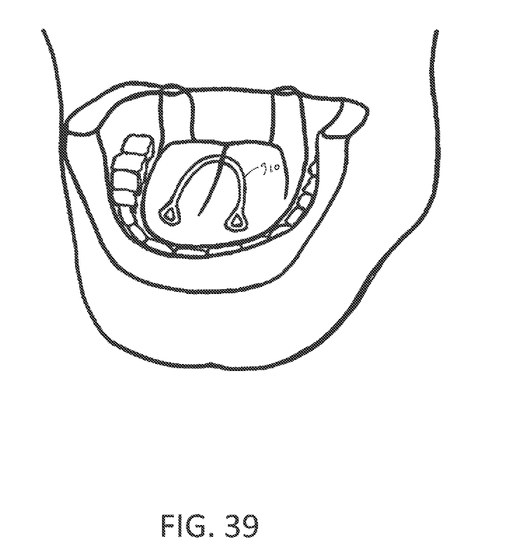

FIG. 39 depicts a curved implant 910 similar to that of FIGS. 38A-38B implanted in a horizontal plane in the patient's tongue. The implant 910 thus partly encircles tissue and applies forces in multiple vectors when stretched to move the tongue forward away from the airway. The implant of FIG. 39 can be implanted using a curved introducer as described previously.

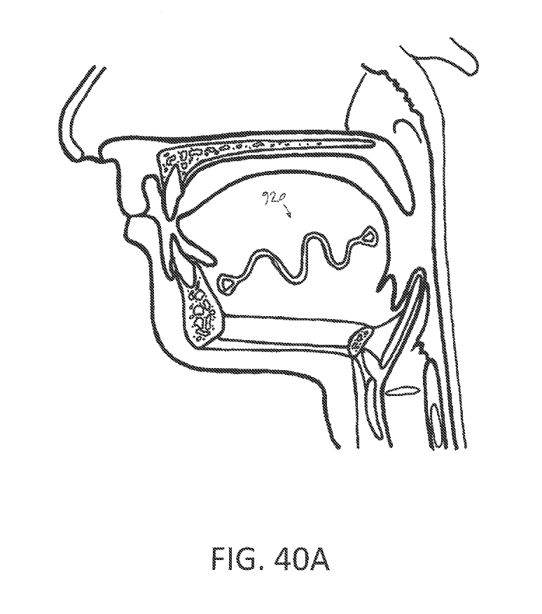

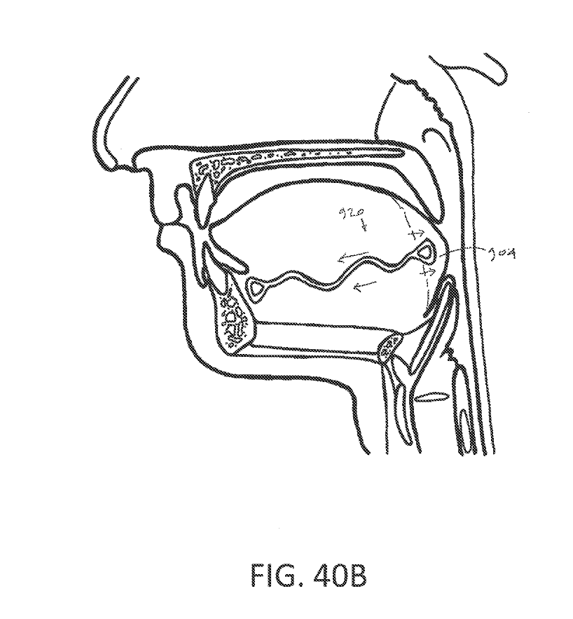

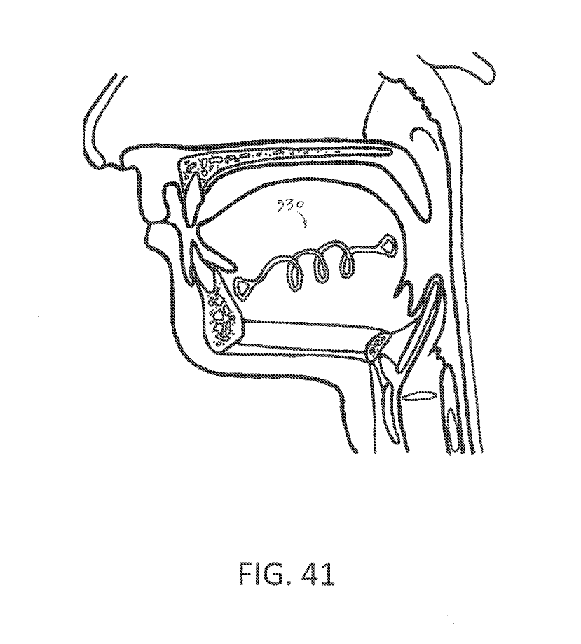

FIGS. 40A-40B depicts another implant 920 that has a serpentine or S-shape in a repose condition in a patient's tongue. As can be understood from FIG. 40B, if the tongue base 904 is displaced posteriorly, the implant will be stretched and the elastic implant will apply forces anteriorly and toward the serpentine condition to compress tongue tissue to prevent airway interference. FIG. 41 depicts another implant 930 that has a helical shape in its repose condition in a patient's tongue. This implant 930 would function as the serpentine implant of FIGS. 40A-40B to apply compressive and anteriorly directed forces to the patient's tongue.

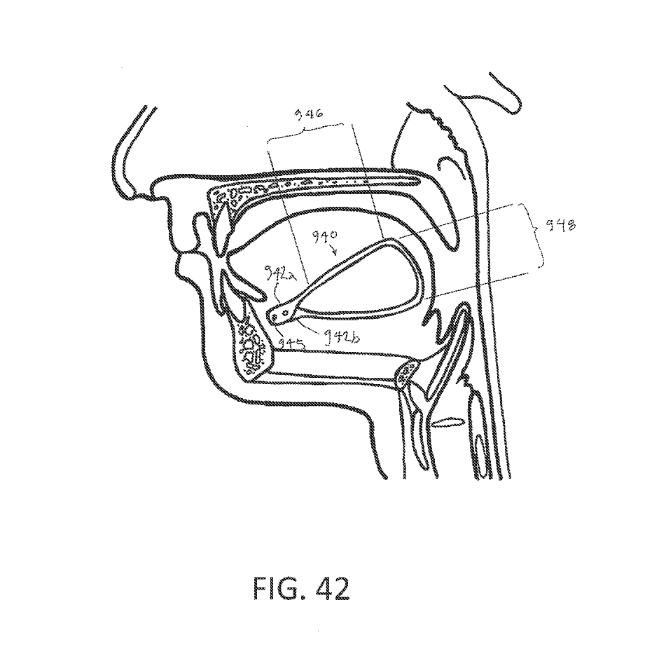

FIG. 42 depicts another type of OSA implant 940 that comprises a loop or tissue-encircling implant at least partly of an elastic material that encircles tongue tissue or other airway-interface. Such an encircling implant 940 can be implanted using introducer systems described further below, wherein first and second end portions 942a and 942b of the implant are coupled by connection means which can be clips, snap-fit features, pins, ratchets, sutures, stakes, clamps, welds, fusible materials, adhesives and the like indicated at 945. The portion between the ends may have a long curvilinear axis, wherein the medial portion is configured to tensile forces along the axis. Such an encircling implant can apply inwardly-directed, elastic and compressive forces on encircled tissue which may cause tissue to remodel to provide a reduced tissue volume. At the same time, the elastic encircling implant will apply forces in a plurality of vectors to return the implant and engaged tissue that is outside the encircling loop toward the repose shape of the implant and engaged tissue within its path in the targeted site. The implant of FIG. 42 can be configured with the bioerodible elements as described previously to allow the forces to be applied to the tissue slowly over a selected time interval. Still referring to FIG. 42, the encircling implant has anterior portion 946 that extends in first and second legs to the cross-over posterior portion 948, wherein the first (anterior) portion 946 has a first elasticity and the second (posterior) portion has a second elasticity. In one embodiment, the anterior implant portion 946 has greater elasticity than the posterior portion 948, and the posterior portion is adapted to distribute applied forces over a region of the tongue. In another aspect, the posterior region may have more than one elasticity.

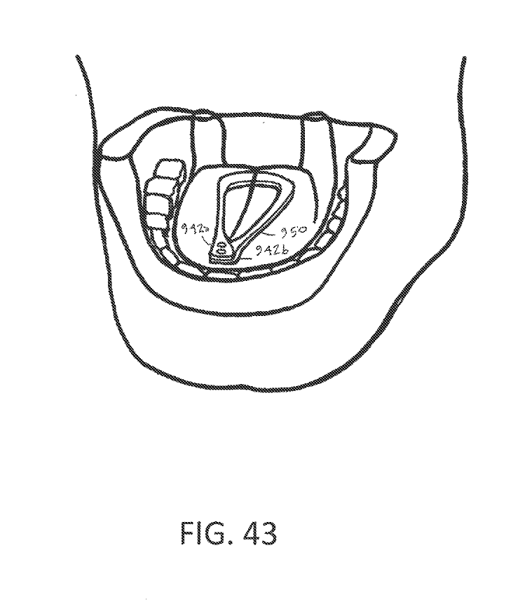

FIG. 43 depicts an encircling OSA implant 950 similar to that of FIG. 42 except that the tissue-encircling implant is placed in a horizontal orientation in the patient's tongue. It should be appreciated that a plurality of encircling implants such as those of FIGS. 42 and 43 can be implanted in a patient.

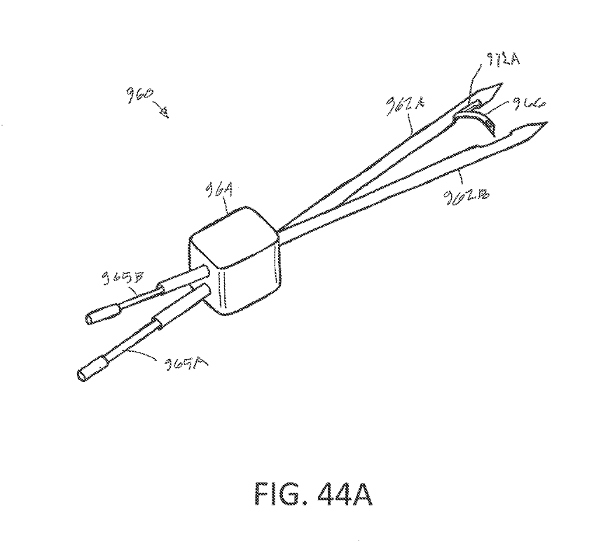

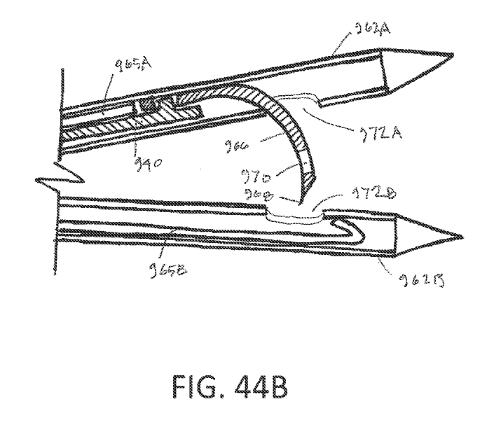

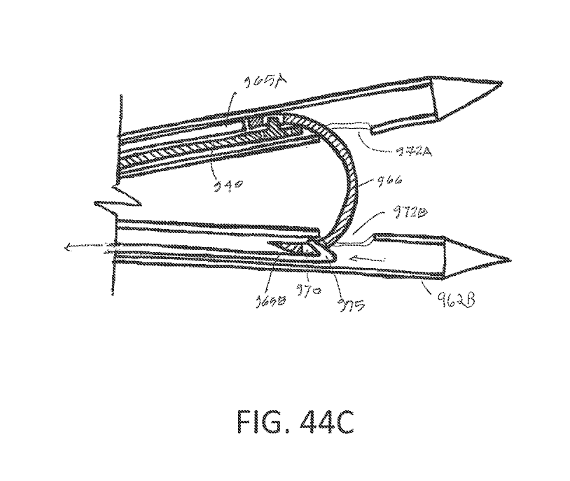

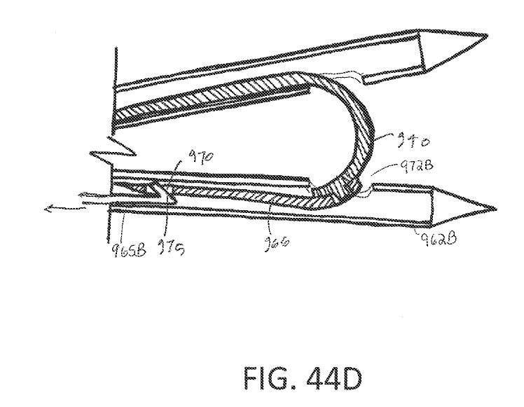

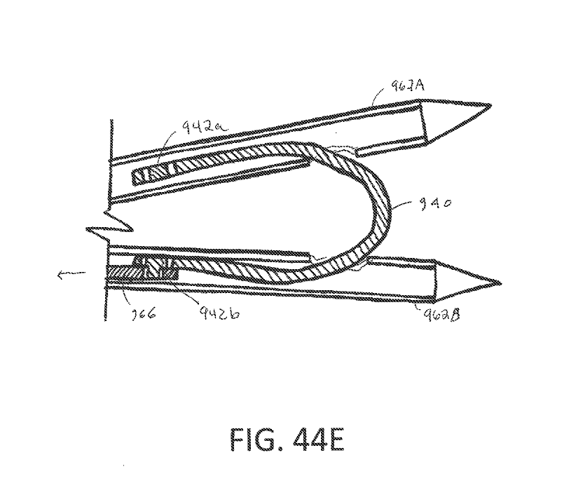

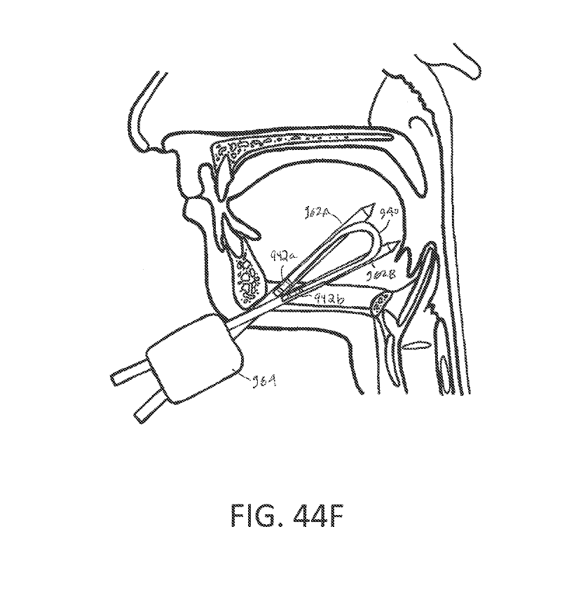

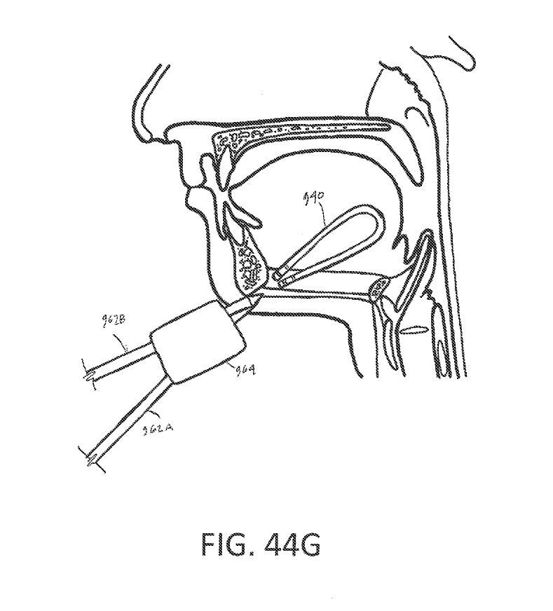

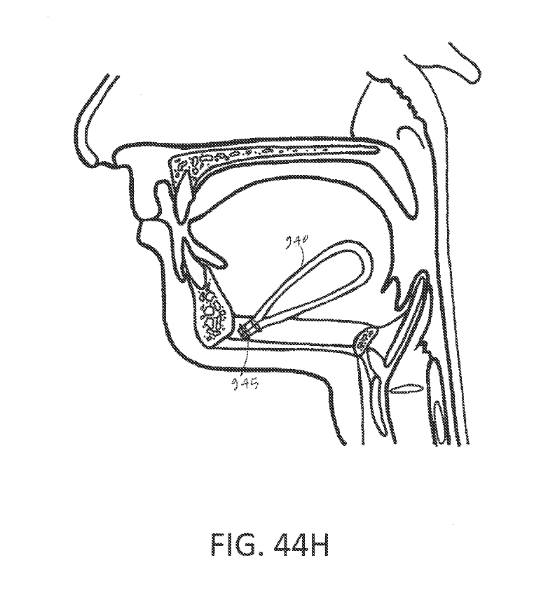

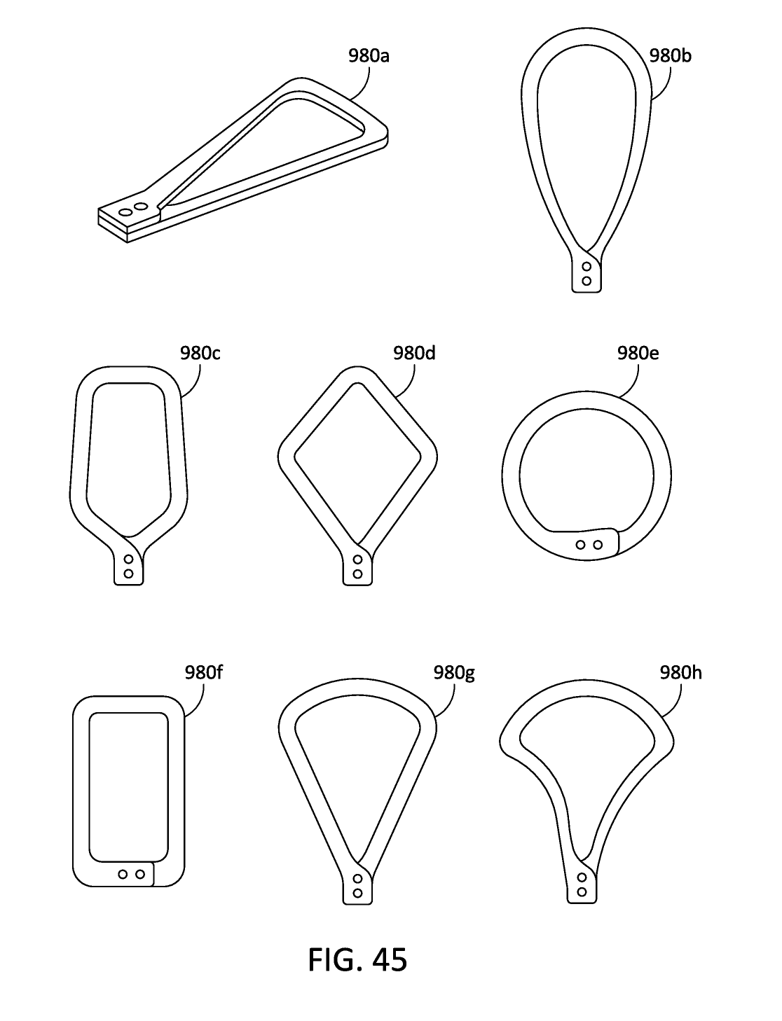

FIG. 44A depicts an introducer system 960 that is adapted for implantation of an encircling-type implant such as the OSA implant of FIG. 42. The introducer system 960 is shown schematically and includes first and second trocar elements, 962A and 962B, a guide block or member 964 which is configured to guide the trocars in a predetermined direction and relative angle when the trocars are extended from the guide block 964 into tissue. Further, the system 960 includes push-pull rods or controlling rods 965A and 965B that are slidably carried in respective bores of the trocar elements, 962A and 962B. In FIGS. 44A and 44B, it can be seen that a releasable, flexible tunneling element 966 that is pre-formed in curve with a sharp tip 968 is releasably coupled to control rod 965A. The distal end of tunneling element 966 is configured with an opening 970 or other grip feature that allows for its coupling to second control rod 965B. The tunneling element 966 has a preformed curvature and can be made of a shape memory alloy (e.g., NiTi) such that when the tunneling element is advanced from the distal port 972A of trocar element 962A, the element tunnels in a curved path to the distal port 972B of the other trocar element 962B.

FIG. 44B depicts a cut-away schematic view of the working end of the system of FIG. 44A in a method of use, wherein the distal portions of the trocar elements 962A and 962B are shown as if advanced from the guide block 965 into a targeted tissue site. FIG. 44B shows the tunneling element moved from retracted position (not shown) in a passageway in trocar element 962A to a first extended position outward of port 972A. It can be seen that an encircling implant 940 of the type shown in FIG. 42 is releasably coupled to tunneling element 966. In some embodiments, coupling is achieved by means of a hook on the tunneling element that holds the implant while the tunneling element and implant advance through tissue. The hook is released upon retraction of the tunneling element. In another embodiment, coupling is achieved by means of a clasp or other means well understood by those of skill in the art. FIG. 44C depicts the next step of the method wherein the curved tunneling element 966 is extended further by advancing rod 965A until the distal end of tunneling element 966 enters port 972B of the opposing trocar element 962B. Thereafter, control rod 965B is moved proximally wherein an engaging hook or other engagement element 975 engages the opening 970 in the tunneling element 966.

FIG. 44D depicts a subsequent step wherein control rod 965B is moved further in the proximal direction and the OSA implant 940 is pulled through the path in tissue created by the tunneling element 966 and then into port 972B of the trocar element 962B. FIG. 44E depicts another step wherein the implant 940 is disposed with ends 942a and 942b fully bridging between the opposing trocar elements 962A and 962B, such that the physician can prepare to withdraw both trocar elements from the tissue site to thereby release the implant and leave the implant in place in the encircling tissue.

Now turning to FIGS. 44F and 44G, the steps relating to FIG. 44E are shown schematically in an optional sub-mandibular access to the patient's tongue. FIG. 44F depicts the implant 940 fully bridged between the trocars 962A and 962B as in FIG. 44E. FIG. 44G shows the trocar elements 962A and 962B withdrawn leaving then implant 940 in place. FIG. 44H then depicts the final step of the method wherein the first and second ends 942a and 942b of the implant 940 are attached to one other by any attachment means 945 as described above of by tissue fibrosis as described above to thereby provide an encircling implant. In one embodiment, implant ends are attached to each another by means of tissue fibrosis. Tissue fibrosis may be induced by having the ends of the implant in sufficiently close proximity to one another such that the fibrotic responses to the implants substantially come in contact with one another. Tissue fibrosis may be induced as a consequence of tunneling (e.g. using trocar or stylet or other means) through the tissue to create a channel through some or all of the gap between the implant ends. The healing response to the channel creates the fibrotic response.

FIG. 45 depicts various shapes and configurations of loop or encircling implants 980a-980h.

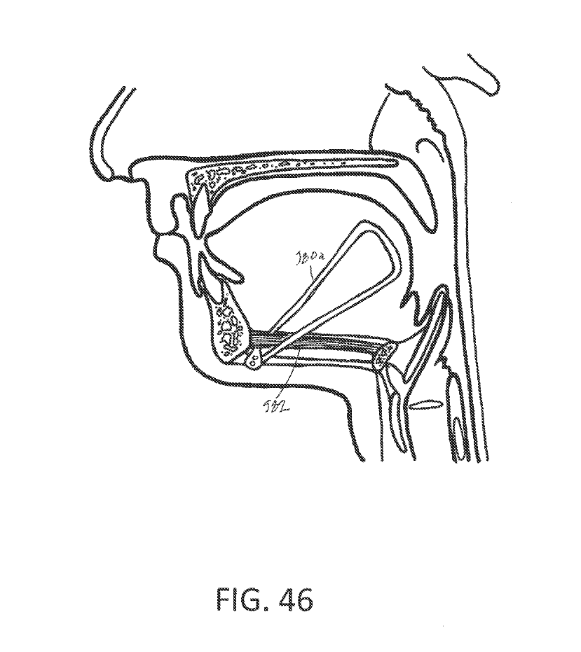

FIG. 46 depicts a loop or encircling implant 980a with its ends fixedly connected around the geniohyoid muscle 982 to serve as an anchor.

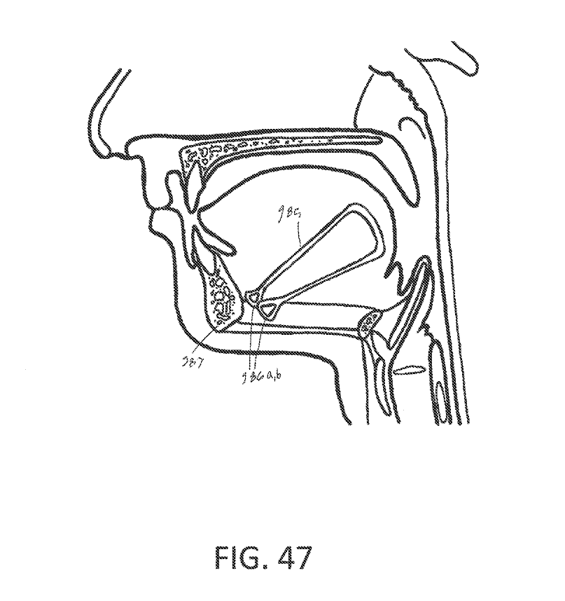

FIG. 47 depicts a U- or V-shaped implant 985 with two anchor ends 986a and 986b as described previously in an anterior position adjacent to the mandible 987. This implant can be placed by the same method as in FIGS. 44A-44H above, except that the ends 986 are not connected in a final step of the method.

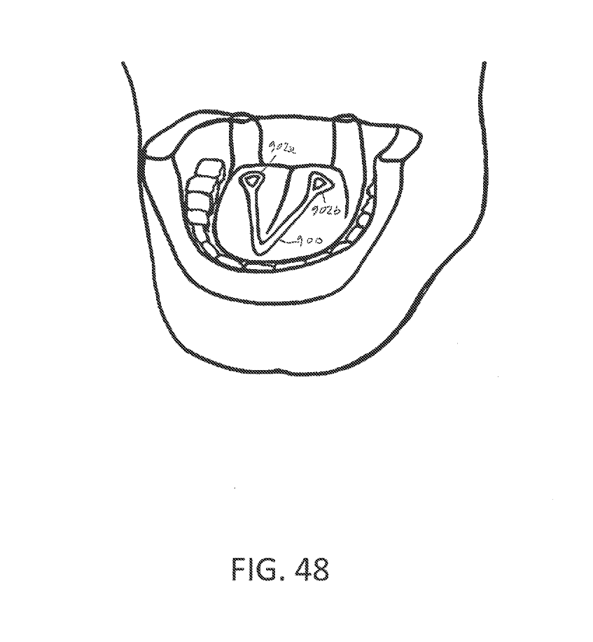

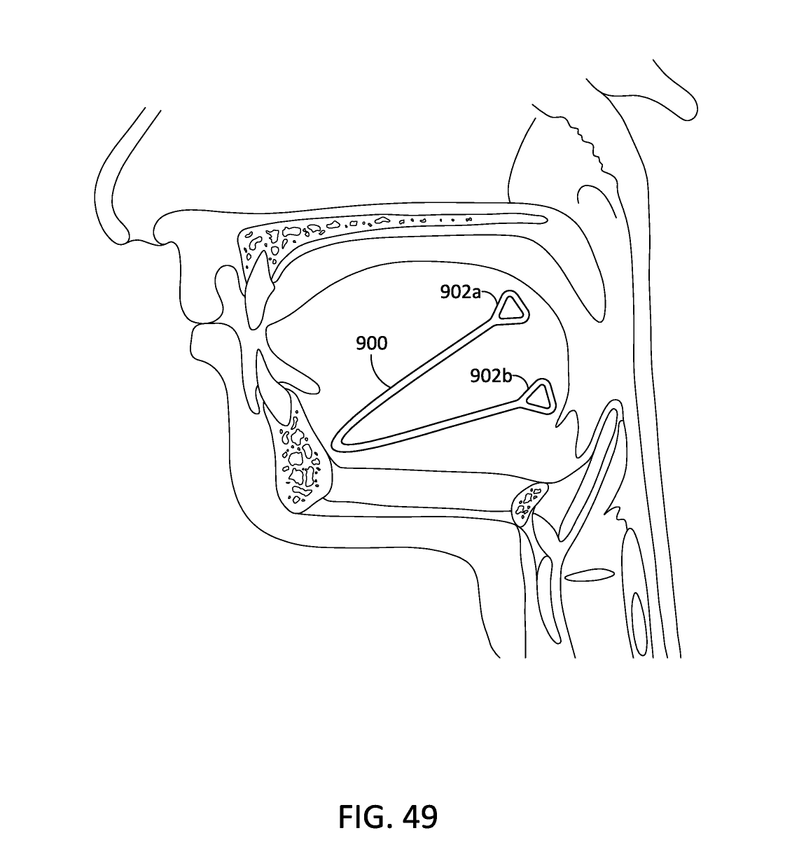

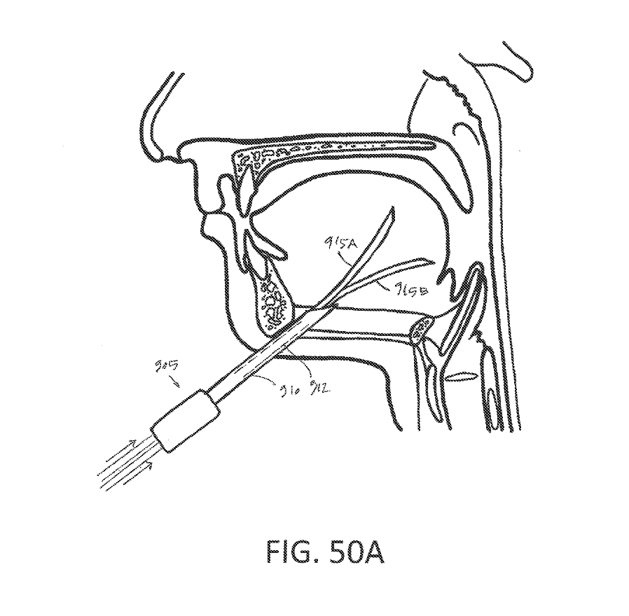

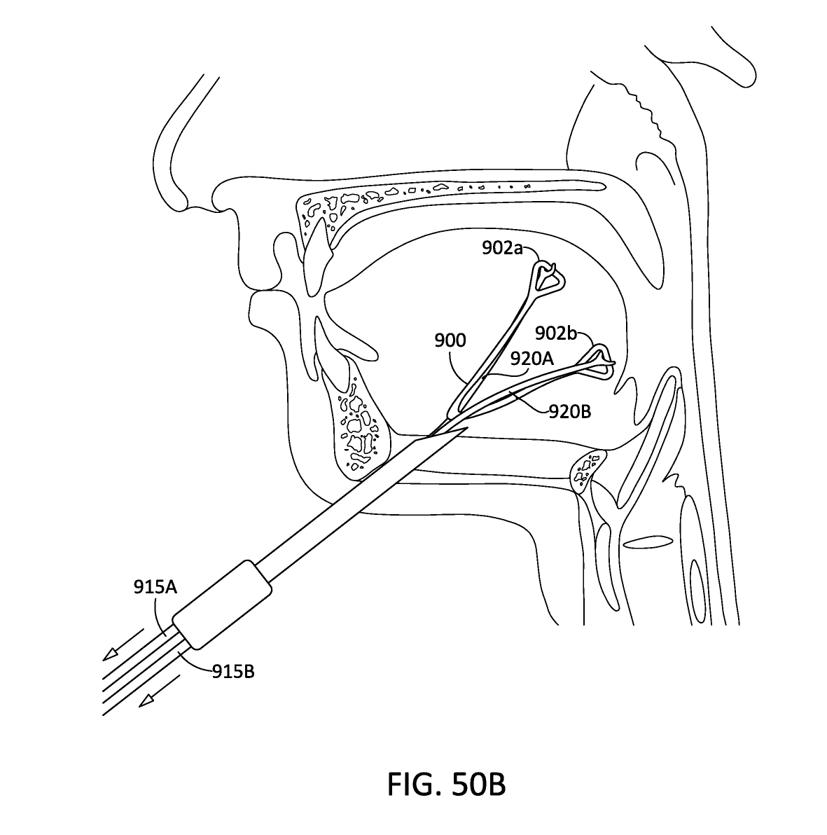

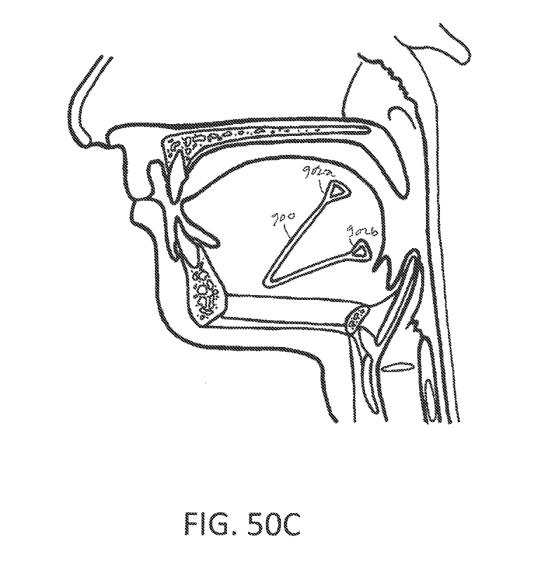

FIGS. 48-49 depict a V-shaped implant 900 with two anchoring portions 902a and 902b at the distal ends of legs of the V-shape. FIG. 48 shows implant 900 in a horizontal orientation, and FIG. 49 shows the implant 900 in a vertical orientation. FIGS. 50A-50C schematically illustrate an apparatus and method for implanting such V-shaped implants through a single entry point. In FIG. 50A, the disclosure provides a trocar 905 with a sharp-tipped trocar sleeve 910 that can be inserted into tissue. A passageway 912 in the trocar sleeve 910 carries first and second curved tunnelers 915A and 915B that can be extended into tissue to form pockets to accept the legs of a V-shaped implant, such as the V-shaped implant 900 that is shown in FIG. 49. A tunneler may have a resilient curved end. A tunneler may be comprised of a shape memory alloy. It can be understood that tunnelers 915A and 915B have a U-shaped transverse sectional shape wherein the longitudinal slot allows for release and deployment of the implant. FIG. 50B depicts the tunnelers 915A and 915B being withdrawn proximally wherein stylets 920A and 920B maintain the implant 900 in the targeted location by grasping implants ends 902a and 902b. FIG. 50C depicts the V-shaped implant 900 in its final deployed location wherein the implant ends 902a, 902b will be anchored in the tissue with tissue plugs as described previously.

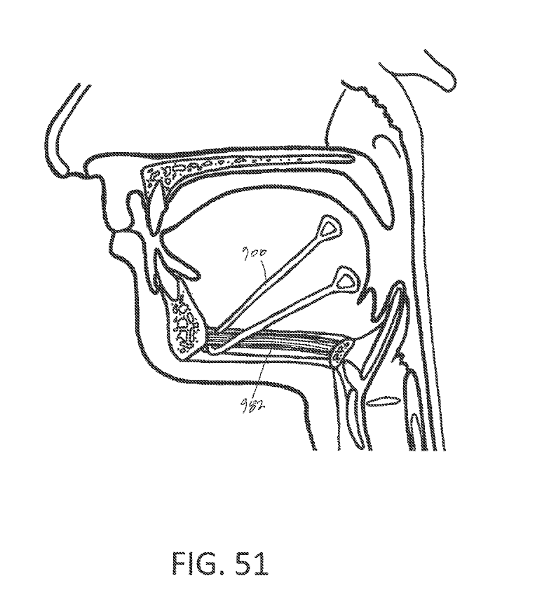

FIG. 51 illustrates a V-shaped implant 900 as in FIGS. 50A-50C anchored around the geniohyoid muscle 982.

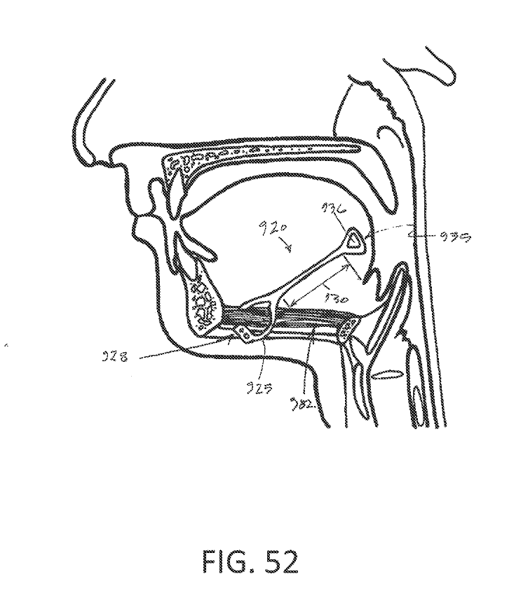

FIG. 52 illustrates an alternative OSA implant 920 that comprises a combination of previously described features wherein the implant includes an encircling portion 925 with attachment means 928 that is coupled to a linear implant portion 930 that extends to an anchoring end 935 that is configured with an opening 936 therein for tissue growth therethrough. The encircling portion 925 encircles the geniohyoid muscle 982.

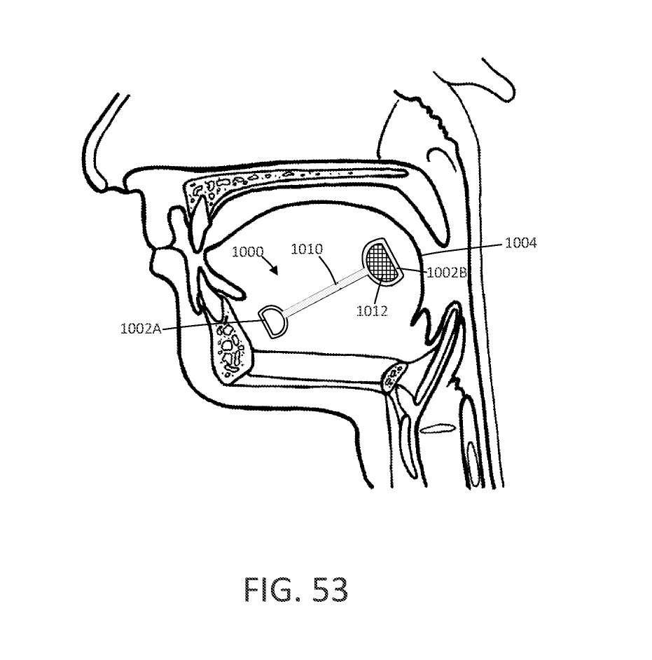

In another aspect of the invention, referring to FIG. 53, an implant 1000 and method are provided for limiting the pressure applied by the implant to the patient's tongue. In FIG. 53, it can be seen that the elongated implant body is configured for treating an airway disorder by implantation in a patient's tongue, wherein a first end portion 1002A of the implant is within an anterior region of the tongue and a second end portion 1002B is in close proximity to a posterior surface 1004 of the tongue. As described in previous embodiments, the medial portion 1010 of the implant body comprises an elastomeric or spring material that is configured to apply tensioning forces to tissue. In this embodiment, the medial portion 1010 can comprise a silicone elastomer or metal spring embedded in a biocompatible elastomer that is designed to provide pressures of less than 20 kPa during normal physiological functioning of the patient's tongue. For clarity, it can be understood that the medial implant portion 1010 will be stretched during tongue function, and the maximum pressure of 20 kPa would thus occur when the implant is stretched to the maximum extent during normal function of the tongue, for example during swallowing. In other embodiments, the medial portion 1010 of the implant 1000 of FIG. 53 can be configured to apply a pressure of less than 15 kPa, less than 10 kPa or less than 5 kPa.

In general, the invention provides a method of treating an airway disorder comprising placing an implant in a patient's tongue wherein the implant has first and second end portions that attach to tissue and a tensioned medial portion between the first and second ends, wherein the medial portion is configured to apply a pressure of less than 20 kPa, less than 15 kPa, less than 10 kPa or less than 5 kPa.

In another aspect of the invention, it is desirable to distribute forces applied by the implant, as in FIG. 53, over a broad area of the tongue to prevent point loads on tissue which could cause tissue dissection, tissue damage or unwanted tissue remodeling. For this reason, referring to FIG. 53 it can be seen that the second end 1002B of the implant body has a planar shape with a cross-section that is substantially larger than the cross-section of the medial extension portion 1010. The planar end portion 1002B can comprise hooks, prongs, loops, mesh, porous structures or any combinations thereof and in FIG. 53 it can be seen that a mesh 1012 is surrounded by a loop element. In one embodiment, the cross-sectional area of the second end is at least 500% of the cross-sectional area of the medial extension portion 1010. In other embodiments, the cross-sectional area of the second end portion is at least 750% of the cross-sectional area of the extension portion, or at least 1,000% of the cross-sectional area of the extension portion.

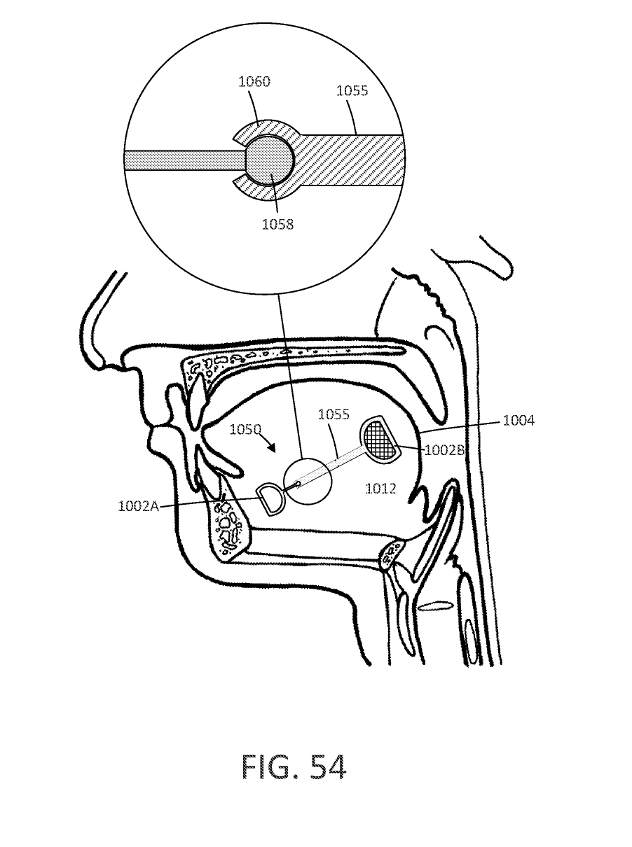

In another aspect of the invention, an implant body 1050 is provided as depicted in FIG. 54 that is configured with a different mechanism to prevent excessive pressures being applied to the tissue, and particularly to the anchoring end portions of the implant body. In the embodiment of FIG. 54, a release mechanism is provided which can include a projecting element 1058 that is gripped by a surrounding grip structure such as polymer flex arms or elements 1060 connected to the second portion of the extension member 1055. It can be understood that under a certain force, the flex arms 1060 can flex to thus release the projecting element 1058. In general, a method for treating an airway disorder comprises providing an elongated implant for implanting in a patient's tongue, wherein the implant comprises an extension member having first and second end portions and an intermediate release element that releases the first end portion from the second end portion upon a preselected pressure on the tongue tissue above the implant, which translates to a force applied to flex arms 1060. The pressure can be less than 20 kPa, less than 15 kPa, less than 10 kPa or less than 5 kPa. In this embodiment, the implant body 1050 will post-failure have the implant with disconnected end, and a minor surgery can be used to revise, remove, re-couple or otherwise adjust the implant.

In another embodiment (not shown) the extension portion of the implant can have a ratchet mechanism that allows the implant to slip between various ratchet elements to thereby adjust the overall length of the implant after when forces exceed a predetermined value as described above.

In another embodiment (not shown) the extension portion of the implant can have a ratchet mechanism that allows for user manipulation to adjust the overall length of the implant. For example, if the implant experiences force requirements greater than a pre-selected level, then the user can manipulate the implant with his fingers to return the first and second end to ratchet toward a shorter overall length of the implant body to allow the implant to apply more force.

In another embodiment, the implant body can be configured with a transponder or RFID type of mechanism which upon an electromagnetic query signal from a remote source, the coil and circuitry in the implant will respond with an electromagnetic answer signal indicating an operational parameter of the implant, for example the implant's length. In another example, the query signals could be periodic or continuous during a patient's sleep to provide information on pressure or force parameters. In one aspect, the invention would be useful for implants that are length-adjustable by the patient, so that the patient can adjust the length before and/or after a sleep interval.

Now turning to FIGS. 53A-53B, other OSA implants 1000 and 1005 are shown wherein each implant body is shown implanted in a patient's tongue and includes an elastic portion that allows for normal physiological function during non-sleep intervals and can apply sufficient retraction forces along implant axis 1008 to alleviate airway obstruction during sleep intervals. More in particular, implant 53A has first and second anchoring end portions 1010a and 1010b that extend about axis 1008 with medial portion 1115 therebetween. The end portions and the medial portion 1115 can comprise a suitable biocompatible elastomer such as silicone. Further, the end portions 1010a and 1010b are configured for anchoring in tissue and thus have openings 1018 or other tissue in-growth features therein as described previously. The medial portion 1115 can be releasably maintained in a stretched configuration during an initial period of tissue in-growth into the end portions 1010a and 1010b as described previously. Of particular interest, the anchoring end portions 1010a and 1010b are flexible but axially non-stretchable or inelastic. The inelastic characteristics of the end portions allows for tissue in-growth to occur more effectively since axial forces are not changing the length of the anchoring end. Further, after the implant is in use to apply retraction forces, each anchoring end portion 1010a, 1010b engages tissue along the entire length of the end portion without greater force being applied to tissue closer to the medial elastic portion 1115, as would be that case if the anchoring end portion was even slightly axially elastic. Any slightly elastic anchoring end could contribute to unwanted tissue remodeling over time.

Referring to FIG. 54, it can be seen that an anchoring end portion 1010a is made axially inelastic by means of non-stretchable reinforcing filaments or elements 1022 embedded therein. Such filaments 1022 can be an inelastic, flexible polymer (e.g., Kevlar), metal wires (e.g. stainless steel, NiTi), carbon fiber or the like. The filaments 1022 can be substantially linear elements or can be knit, woven or braided structures as in known in the art. As can be understood from FIG. 54, the end portion 1010a is thus axially inelastic but is still flexible and twistable relative to axis 1008.

FIGS. 53A and 53B further illustrate that the anchor portion's axial length of AL can have a selected relationship to the medial portions axial length AM, and thus the overall implant length which is dependent on the desired amount of axial retraction forces applied by the implant. For example, in FIG. 53A, in one embodiment each anchoring end length AL can be 15% of the overall length of the implant which has a medial portion 1115 configured to apply a retraction force of 3.0 Newtons. FIG. 54A depicts another embodiment wherein each anchoring end length AL′ can be 35% of the overall length of the implant and the medial portion 1115 with length ML′ can still be configured to apply a retraction force of 3.0 Newtons. In this embodiment, the design in FIG. 53B may be preferred because of the increased anchoring length, which would decrease the likelihood of tissue remodeling over time.

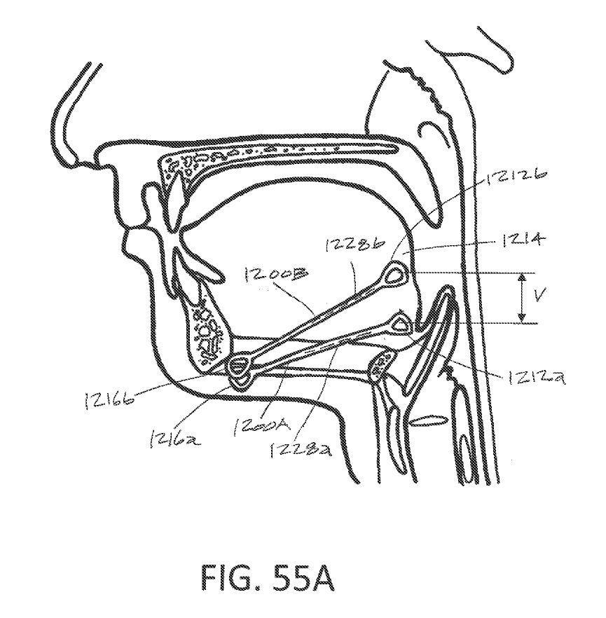

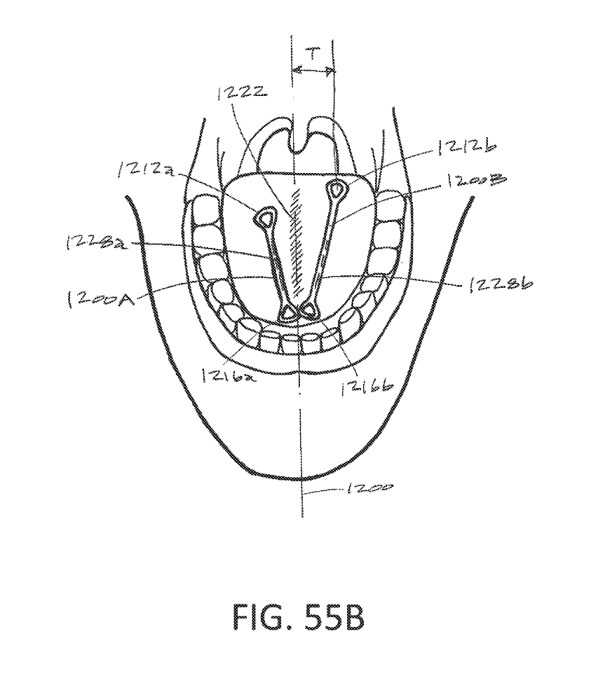

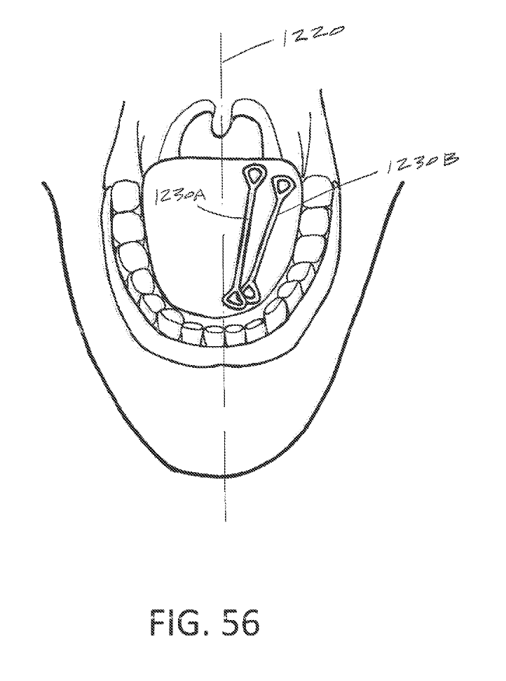

FIG. 55 illustrates a single implant 1005 of the type shown in FIGS. 53A-53B implanted in a patient's tongue wherein the anchoring end portions 1010a, 1010b have an axial length AL′ suited for a particular tissue site, for example close to the base of the tongue and close to the mandible. These end lengths may be the same or may vary, and multiple implants may be used as depicted schematically in FIG. 56. For example, multiple implants in FIG. 56 can collectively apply a selected retraction force and may be used instead of one implant to apply the desired force—but with less force applied per implant 1005, which can reduce remodeling forces applied to any single anchoring end portion.

In general, an implant according to the invention for treating an obstructive airway disorder comprises an elongated implant body having an axis and configured for implanting in airway-interface tissue, wherein the implant body has a medial portion extending between first and second anchoring end portions and wherein the medial portion is axially complaint and the end portions are axially non-compliant. The anchoring end portions are configured for tissue growth therein or therethrough yet allow normal physiological function during non-sleep and sleep intervals. The implant end portions comprise an elastomer with an embedded non-stretch structure. The medial portion 1115 can comprise an elastomer or an elastomer with an embedded helical spring element. The implant can be configured for implantation in the epiglottis, soft palate, pharyngeal wall or tongue tissue.

In one embodiment, the implant has a medial portion extending between the first and second anchoring end portions, wherein each end portion has an axial length of least 15%, 20%, 25%, 30%, 35% or 40% of the overall length of the implant in a repose state of the overall length of the implant. The implant end portions can each have an axial length of a least 4 mm, 6 mm, 8 mm, 10 mm or 12 mm.

In another embodiment, the implant has a medial portion extending between the first and second anchoring end portions, wherein the medial has an axial length of least 40%, 50%, 60% or 70% of the overall axial length of the implant.

FIGS. 55A-55B illustrate another method of treating an airway disorder which comprises implanting two elongated implants 1200A and 1200B similar to those described above in a patient's tongue 1204 in a non-parallel orientation. In the side view of FIG. 55A, it can be seen that the anterior ends 1206a and 1206b of the implants 1200A and 1200B, respectively are anchored proximate the patient's mandible 1208. The anterior ends can be fastened directly to the mandible or implanted in tissue adjacent the mandible. In another variation, the anterior ends can be coupled to each other or coupled to one another and slidably coupled to an anchor in the mandible.

In FIGS. 55A and 55B, it can be seen that the posterior ends 1212a and 1212b of implants 1200A and 1200B, respectively, are positioned in a posterior region of the base 1214 of the patient's tongue. As can be seen in FIGS. 55A-55B, one variation of a method corresponding to the invention comprises implanting the two elongated implants in the tongue wherein the orientations of the implant axes are non-parallel. In particular, the posterior ends 1212a and 1212b of implants 1200A and 1200B are spaced apart vertically by a selected dimension V which can be at least 0.25 cm, at least 0.50 cm, at least 1 cm or at least 1.5 cm. In one variation, the spacing indicated at V in FIG. 55A can be between 1 cm to and 1.5 cm. Referring to FIG. 55B, the implants 1200A and 1200B can be on opposing sides of the mid-line 1220 of the tongue with the anterior implant ends 1216a and 1216b close to the mid-line 1220 and the posterior ends 1212a and 1212b spaced transversely from the mid-line 1220 a distance T that can range from 0 to 1 cm. The implants can lie on opposing sides of the median longitudinal raphe 1222 of the tongue. For example, the implants may be on opposite sides of a sagittal plane of the patient, and in particular may be on opposite sides of a mid-sagittal plane (a longitudinal plane that divides the body into left and right sections). In this variation, it has been found that restraint provided by the implants over a vertical region of the base of the tongue can assist in preventing airway obstruction. In all other respects, the implants depicted in FIGS. 55A-55B can be the same or similar to the implants described earlier in this disclosure, with all, some, or none of the features. For example, the implants may have an expanded configuration and a contracted configuration and may be held in the expanded configuration, such as by a bioerodible portion.

In general, a method includes implanting first and second elongated implants in a patient's tongue, wherein each implant has an anterior end in an anterior location and a posterior end in a posterior location in the patient's tongue, and wherein the posterior end locations are asymmetric relative to a transverse plane. Further, each implant may be asymmetric relative to the mid-line of the tongue.

A method of treating an airway disorder or otherwise treating airway, mouth, nasal, or throat tissue may include implanting at least first and second elongated implants in a tongue of a patient, wherein each of the first and second implants is configured to have a first, expanded configuration and a second, contracted configuration, wherein implanting comprises implanting the first and second implants having their first, expanded configurations, and wherein each implant has an anterior end in an anterior location and a posterior end in a posterior location in the patient's tongue and the posterior end locations are different vertical distances from a transverse plane of a patient. The implants may have a bioerodible portion and an elastomeric portion, and the method may include holding the respective elastomeric portion of each implant in the first expanded configuration with the respective bioerodible portion of the implant.

Another method of treating an airway disorder comprises implanting at least first and second elongated implants in a patient's tongue wherein each implant has an axis and wherein the first axis of 1228a of the first implant 1200A and the second axis 1228b of the second implant 1200B are non-parallel relative to the mid-line 1220 of the tongue (FIG. 55B). Further, the first axis 1228a and the second axis 1228b of the implants may be asymmetric relative to a transverse plane (FIG. 55A).

Another method of treating an airway disorder or otherwise treating airway, mouth, nasal, or throat tissue may include implanting at least first and second elongated implants in a tongue of a patient, wherein each implant is configured to have a first, expanded configuration and a second, contracted configuration and implanting comprises implanting the first and second implants in their first expanded configurations, and wherein each implant has an axis and wherein the axis of the first implant and the axis of the second implant are oblique relative to at least one of a midline plane of the tongue and a transverse plane of the tongue. In a particular embodiment, the axis of the first implant and the axis of the second implant may be oblique relative to both the midline plane of the tongue and the transverse plane of the patient.

FIG. 56 illustrates another implant configuration and method for treating an airway disorder which comprise implanting a plurality of axially-extending implants in a patient's tongue wherein the implants are disposed on one side of the patient's mid-line. For example, in FIG. 56, implants 1230A and 1230B are disposed on one side of the mid-line 1220 of the tongue.

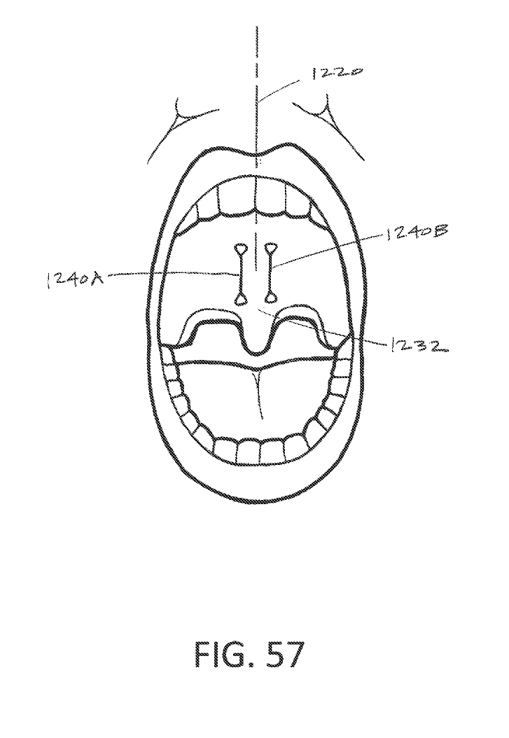

FIGS. 57-61 illustrate variations of methods for treating an obstructive airway disorder relating to implanting at least first and second elongated implants in a patient's soft palate 1232. The implants can be of the types described above which include anterior and posterior anchoring ends and an elongated resilient medial region. FIG. 57 illustrates implants 1240A and 1240B which are implanted in the soft palate 1232 with each implant axis extending between the anchoring ends being symmetric and parallel relative to the patient's mid-line 1220. In general, the palate implants have a length of about 2.5 cm to 3.0 cm.

A method of treating an obstructive airway disorder or otherwise treating airway, mouth, nasal, or throat tissue may include implanting at least first and second elongated implants in a patient's soft palate, each implant having anchoring ends and configured to have a first, expanded configuration and a second, contracted configuration, and implanting comprises implanting the implants each having a first, expanded configuration, each implant further having an axis extending between its anchoring ends, wherein the axis of the first implant and the axis of the second implant are symmetric relative to a mid-line of the patient.

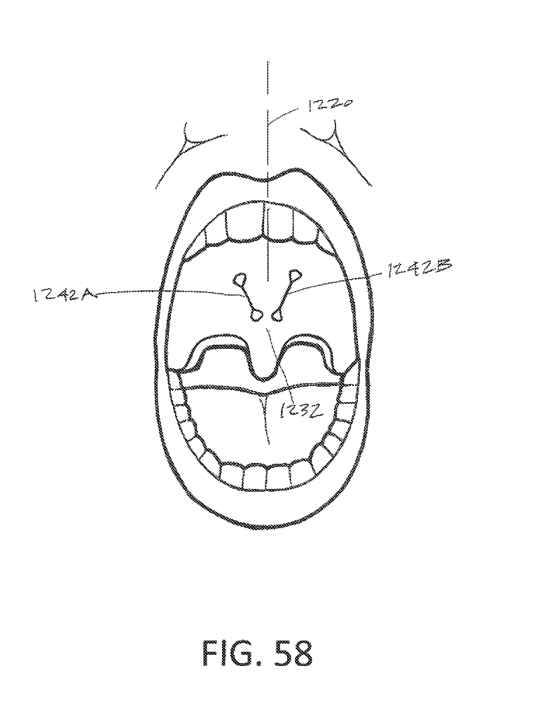

FIG. 58 illustrates another variation in which implants 1242A and 1242B are implanted in the soft palate 1232 with the implant axes being symmetric relative to the mid-line 1220 but converging in the posterior direction in the soft palate.

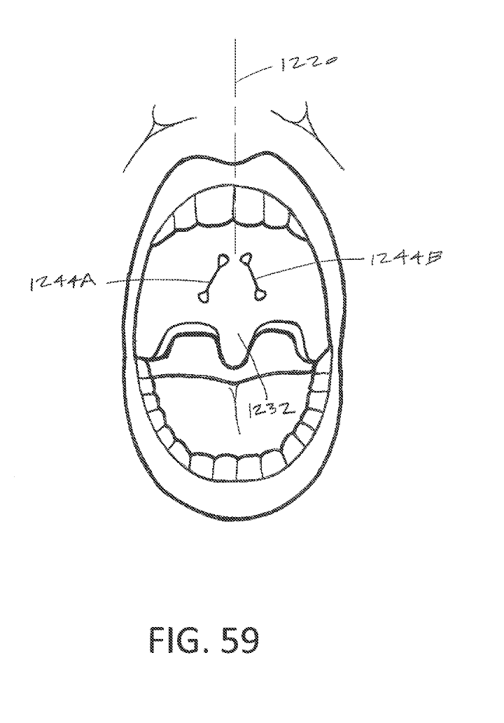

The variation of FIG. 59 is similar to that of FIG. 58 except the implants 1244A and 1244B in the soft palate 1232 have axes that are symmetric relative to the mid-line 1220 but diverge in the posterior direction in the soft palate.

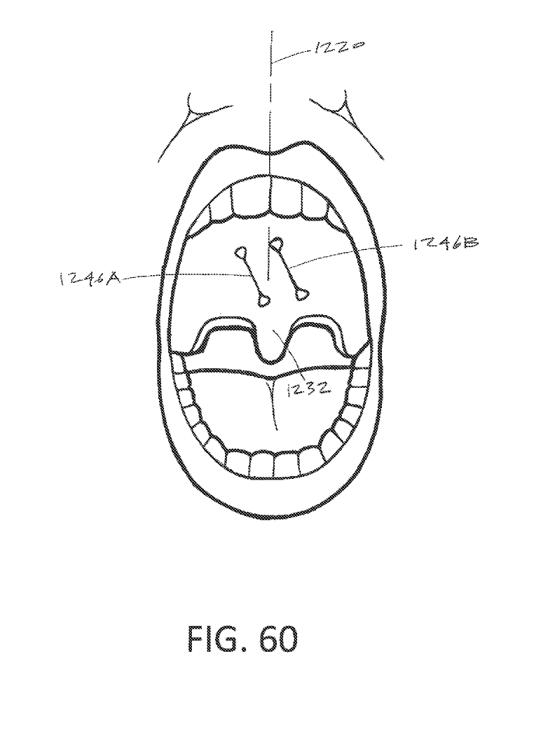

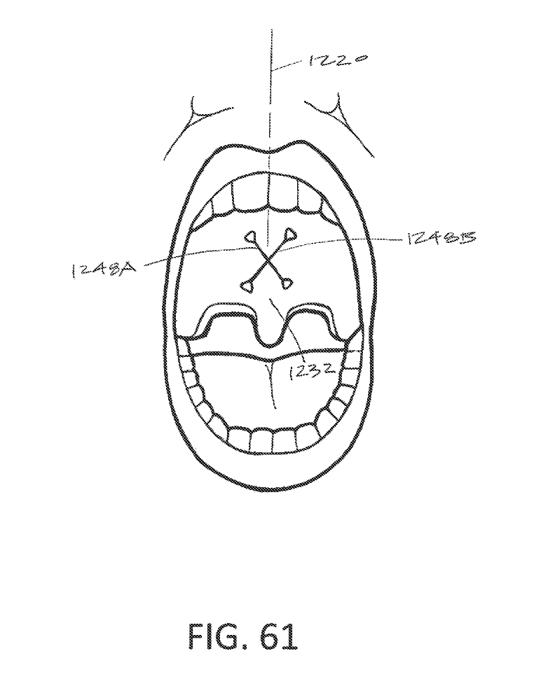

FIG. 60 illustrates another variation in which first and second implants 1246A and 1246B are implanted in the soft palate 1232 with axes that are parallel with each other but have an angled orientation relative to the mid-line 1220. The variation of FIG. 61 depicts first and second implants 1248A and 1248B implanted in the soft palate 1232 with axes that cross one another and are angled relative to the mid-line 1220. Implants that cross one another may contact each other or may cross over one another (e.g. may appear to cross each other if viewed from the top (head) of the patient).

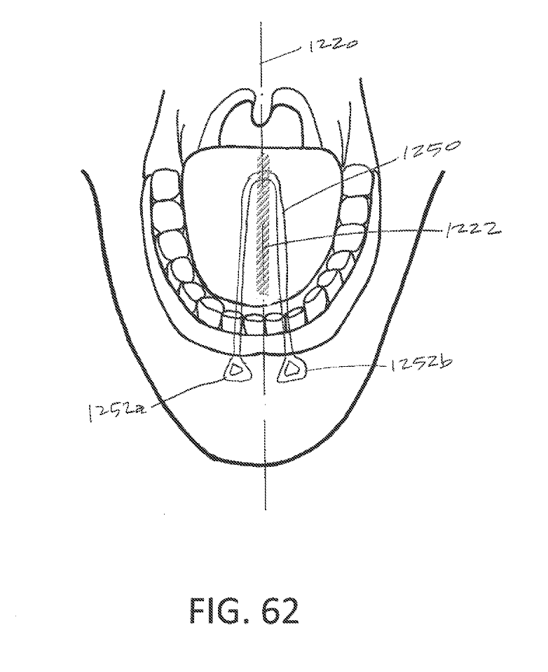

FIG. 62 represents another method of the invention which includes utilizing at least one implant having a posterior portion that extends through the median longitudinal raphe 1222 of the tongue. In this variation, the median raphe is believed to provide a more durable tissue region against which forces can be applied by an implant body which can result in less implant migration and a lower potential of tissue remodeling which also can reduce the effectiveness of the implant(s). In one embodiment depicted in FIG. 62, the implant 1250 can comprise one or more components and the implant may have first and second anterior ends 1252a and 1252b anchored near the patient's mandible as described previously. In this variation, the median raphe 1222 is penetrated in a single location but the implant system can also provide multiple penetrations. In some variations, the median raphe 1222 can be penetrated in two or more locations spaced apart vertically as generally indicated in the implant configuration of FIGS. 55A-5B.

In general, a method of treating an obstructive airway disorder comprises implanting at least one implant in a patient's tongue wherein a posterior anchoring end of the implant extends through the median longitudinal raphe of the tongue. The method can include providing an implant with an anterior anchoring end or ends proximate the patient's mandible. The method of treating an obstructive airway disorder can include implanting at least one implant in a patient's tongue wherein first and second portions of the implant extend through the median longitudinal raphe of the tongue.

Placing multiple implants in a patient may provide better tongue or other tissue remodeling, better tongue or other tissue control, fewer side effects and/or may allow smaller implants to be placed. Multiple incisions may be made and used to place implant(s) or two or more implants may be placed through a single incision. Another method of implanting an implant or treating a treating an airway disorder or otherwise treating airway, mouth, nasal, or throat tissue may include creating a surface incision on a surface of a tissue near an airway forming tissue, placing a delivery device holding a first elongate implant at least partially through the incision and into the airway forming tissue, placing the first elongate implant into a first position in the airway forming tissue, removing the delivery device from the airway forming tissue wherein the first elongate implant remains in the airway forming tissue, placing a second delivery device holding a second elongate implant through the incision and into the airway forming tissue, placing the second elongate implant into a second position in the airway forming tissue, and removing the second delivery device from the airway forming tissue wherein the second elongate implant remains in the airway forming tissue. A surface incision may be any size required but preferably is very small. An incision may be less than 3 cm, less than 2.5 cm, less than 2 cm, less than 1.5 cm, less than 1 cm, or less than 0.5 cm in a widest dimension. Placing the first implant may include placing it on one side of a midline of a tongue and placing the second implant may include placing it on the other side of the midline of the tongue.

If the first implant has a first axis forming a first angle with a transverse plane of the patient and the second implant has a second axis forming a second angle with the transverse plane of the patient, placing the first and second implants may include forming oblique angles between the first and second axes and the transverse plane. If the first implant has a first axis forming a first angle with a midline plane of the tongue and the second implant has a second axis forming a second angle with the midline plane of the tongue, wherein placing the first and second implants comprises placing each implant axis at an angle oblique to the midline plane. In some embodiments, the same delivery device may be used to place the first and second (or more) implants. In some embodiments, different delivery devices may be used to place the first and second (or more) implants.

In general, a method for treating an airway disorder comprises implanting an implant body into airway-interface tissue wherein the implant body is sized and shaped to conform in a manner compatible with normal physiological function of the site and to apply selected forces to the tissue, and wherein the implant is configured to receive an electromagnetic query and to respond with an electromagnetic signal indicating an operational parameter of the implant body during said normal physiological function of the site.

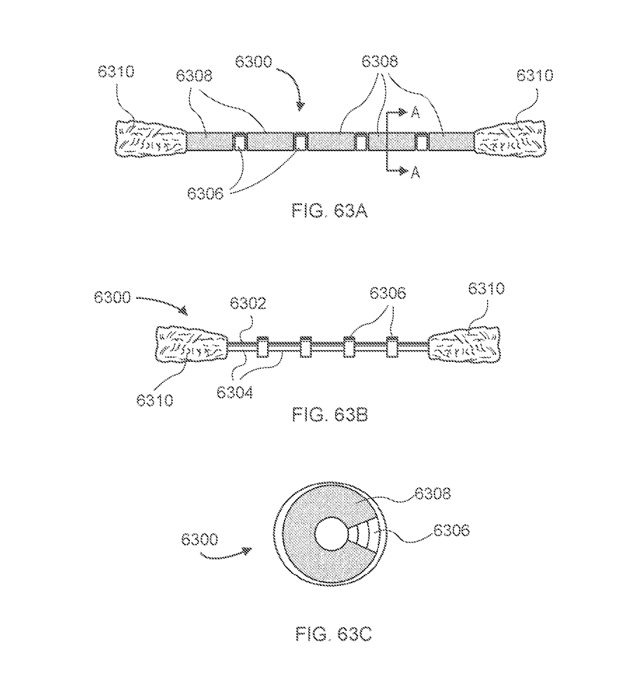

FIGS. 63A-C show one embodiment of a device 6300 that may be implanted in airway-forming tissue to maintain patency of the patient's airway. Device 6300 has a body 6302 with a plurality of narrow sections 6304 separated by wide sections 6306. As shown, the narrow and wide sections are cylindrical, although other shapes may be used. The body 6302 may be made of a resiliently deformable material, such as silicone rubber, polyurethanes or other resiliently deformable polymer or a coil of stainless steel, spring steel, or superelastic nickel-titanium alloy or other resiliently deformable metal, or a composite of the resiliently deformable polymer and metal.

FIG. 63B shows body 6302 in its at-rest shape. In FIG. 63A, body 6302 has been stretched to a deformed shape. Spacers 6308 formed from a bioerodable or bioabsorbable material (such as, e.g., polycaprolactone, polylactic acid, polyglycolic acid, polylactide coglycolide, polyglactin, poly-L-lactide, polyhydroxalkanoates, starch, cellulose, chitosan, or structural protein) have been inserted between wide sections 6306 to maintain the device in its deformed shape. In this embodiment, the spacers 6308 are injection molded and have a C shape, although other manufacturing techniques and other shapes may be used as desired.

Anchors 6310 are formed at both ends of body 6302. In this embodiment, anchors 6310 are formed from a non-woven fabric (such as polypropylene, polyethylene, or polyester) to promote tissue ingrowth. Other anchors may be used, as desired.

Device 6300 may be implanted in a patient's airway-forming tissue in the deformed shape shown in FIG. 63A. In some embodiments, the device 6300 is not affixed to the airway-forming tissue when implanted. Over time, tissue may grow into the fabric of anchors 6310 to at least partially affix the device to the airway-forming tissue. Also over time, the bioerodable spacers 6308 will bioerode, thereby permitting device 6300 to move back toward the at-rest form shown in FIG. 63A. As it attempts to return to its at-rest shape, device 6300 exerts a force on the airway-forming tissue into which it is implanted to maintain the patient's airway in a patent condition.

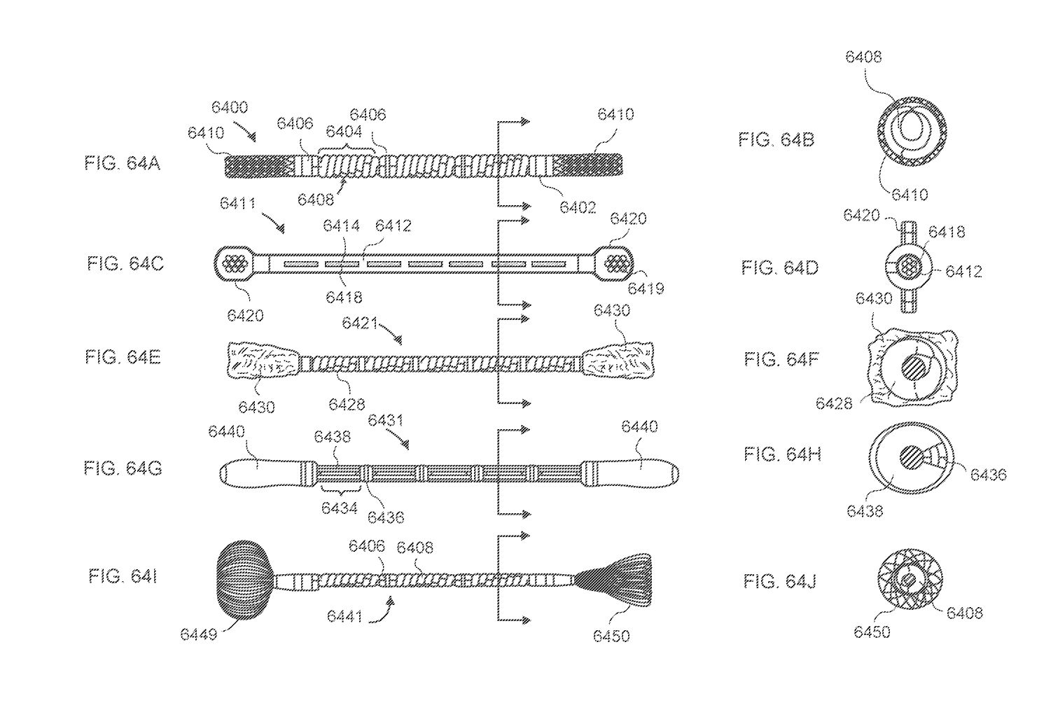

FIGS. 64A-J show various other embodiments of the invention in their deformed states. As in the embodiment of FIG. 63, these devices for maintaining patency of an airway may be implanted into airway-forming tissue of the patient in the illustrated deformed state. Over time, tissue may grow into the device anchors and possibly other parts of the device to at least partially affix the device to the airway-forming tissue. Also over time, the bioerodable spacer portions of the device may bioerode, thereby permitting the device to attempt to move toward a shorter at-rest shape, thereby exerting a force on the airway-forming tissue into which it is implanted to maintain the patient's airway in a patent condition. The deformable bodies of these devices may be formed, e.g., of silicone rubber.

In FIGS. 64A-B, device 6400 has a stiff bioerodable fiber 6408 helically wound within narrow sections 6404 of a resiliently deformable body 6402 between wide sections 6406 to maintain body 6402 in its stretched deformed state. Fiber 6408 may be made, e.g., of polyglactin 910, which is a copolymer of 90% glycolide and 10% L-lactide. When fiber 6408 bioerodes, body 6402 will attempt to shorten to its at-rest shape. Anchors 6410 are disposed at both ends of body 6402. Anchors 6410 may be formed from woven polyester, polyethylene or polypropylene to provide for tissue ingrowth.

FIGS. 64C-D show a device 6411 having a resiliently deformable body 6412 in which a plurality elongated openings 6414 are formed. In the depicted deformed state, bioerodable, rod shaped, spacers 6418 (formed from, e.g., polylactidecoglycolide (PLG)) are disposed in the openings 6414 to maintain the body's elongated deformed shape. Paddle-shaped anchor regions 6420 having a plurality of holes or depressions 6419 are disposed at both ends of body 6412. Holes or depressions 6419 permit tissue in-growth. Anchor regions 6420 may be integral with the central portion of body 6412 or may be formed from a different material, such as reinforced polyester. Anchor regions also may be integral with the central portion of body 6412 and contain a composite reinforcing element such as a polyester fabric.

FIGS. 64E-F show a device 6421 similar to that shown in FIGS. 63A-C in which the bioerodable portion 6428 is formed of a helically wound bioerodable fiber, such as that discussed above with respect to FIGS. 64A-B and contains anchoring regions 6430 of non woven fabric (e.g. polyester, polyethylene, or polypropylene).

FIGS. 64G-H show a device 6431 having a resiliently deformable body 6432 similar to body 6402 of FIG. 64A. As shown, body 6432 is in a stretched deformed shape. Bioerodable spacers 6438 (similar to those of the embodiment shown in FIG. 63A) are disposed in narrow portions 6434 between wide portions 6436 to maintain body in this stretched shape. Anchors 6440 on both ends are formed from an open or closed cell foam material to promote tissue in-growth.

FIGS. 64I-J show a device 6441 substantially the same as the device shown in FIGS. 64E-F with the exception of the anchors 6449 and 6450. In this embodiment, anchors 6449 and 6450 are self-expanding baskets that can be compressed to the form shown as anchor 6450 during implantation and will self-expand toward the at-rest shape shown as anchor 6449 after deployment. The open areas of the anchors provide material loops and spaces for tissue ingrowth and attachment.

Other embodiments of the airway maintaining device may use various aspects of the illustrated embodiments as needed. For example, the anchors at end of the device body may differ from each other.

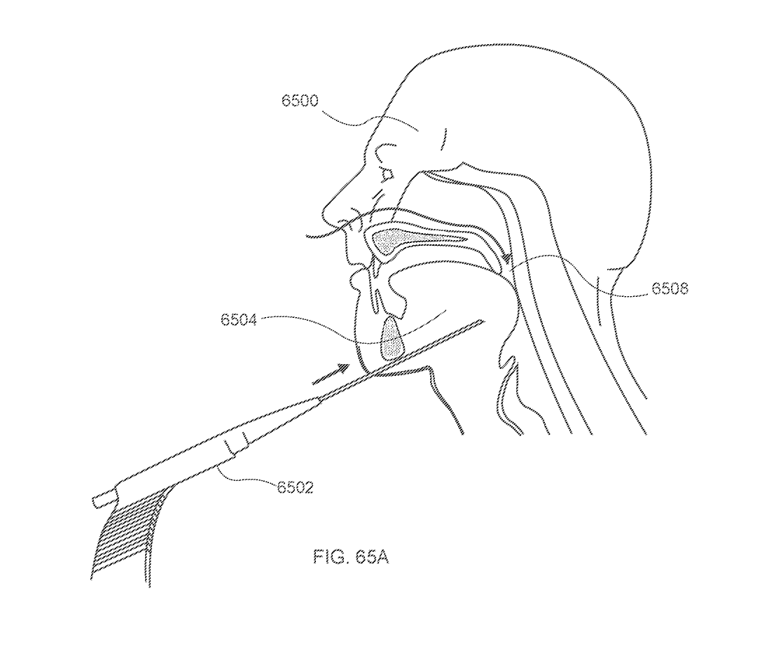

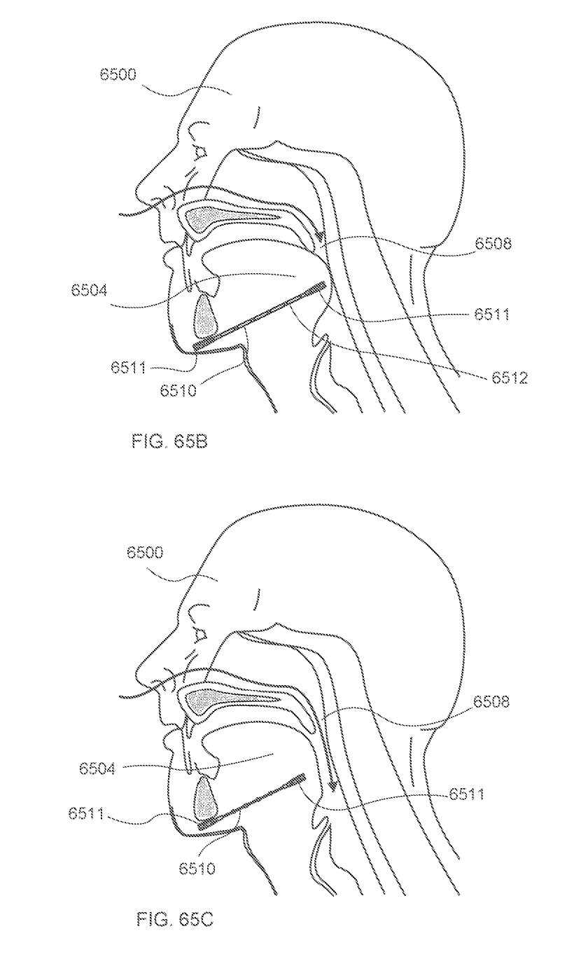

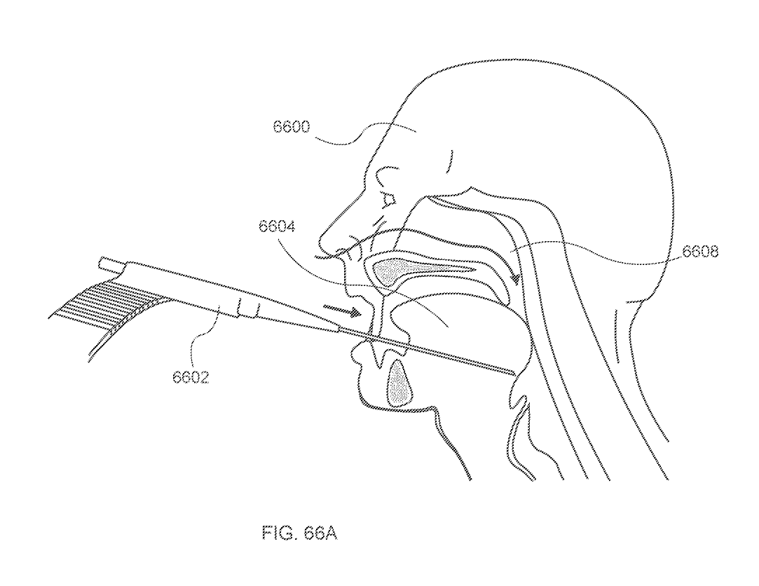

FIGS. 65-67 illustrate therapy provided by embodiments of this invention. In FIGS. 65A-C, a delivery tool 6502 has been inserted submandibularly into the patient 6500 to deliver an airway maintaining device 6510 into a region of the patient's tongue 6504 forming part of the patient's airway 6508, which is shown as being blocked in FIG. 65A. Device 6510 may be, e.g., any of the devices discussed above with respect to FIGS. 63 and 64. As shown in FIG. 65B, the device 6510 is delivered in an elongated deformed state. In some embodiments, device 6510 when first delivered is not affixed to the tongue tissue. Over time, however, tissue may grow into the anchors 6511 of device 6510 and/or other parts of the device. Also over time, bioerodable portions 6512 of device 6510 will bioerode, thereby permitting device 6510 to move toward a shorter at-rest shape, thereby applying a force to the patient's tissue to maintain the patency of the airway, as shown in FIG. 65C.

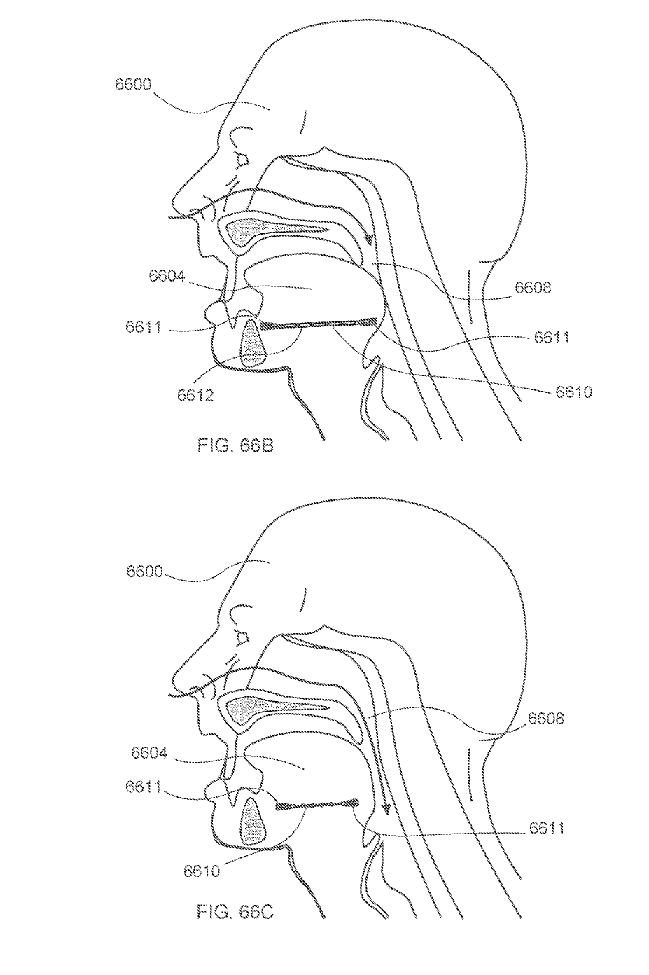

In FIGS. 66A-C, a delivery tool 6602 has been inserted intraorally and sublingually into the patient 6600 to deliver an airway maintaining device 6610 into a region of the patient's tongue 6604 forming part of the patient's airway 6608, which is shown as being blocked in FIG. 66A. Device 6610 may be, e.g., any of the devices discussed above with respect to FIGS. 63 and 64. As shown in FIG. 66B, the device 6610 is delivered in an elongated deformed state. In some embodiments, device 6610 when first delivered is not affixed to the tongue tissue. Over time, however, tissue may grow into the anchors 6611 of device 6610 and/or other parts of the device. Also over time, bioerodable portions 6612 of device 6610 will bioerode, thereby permitting device 6610 to move toward a shorter at-rest shape, thereby applying a force to the patient's tissue to maintain the patency of the airway, as shown in FIG. 66C.

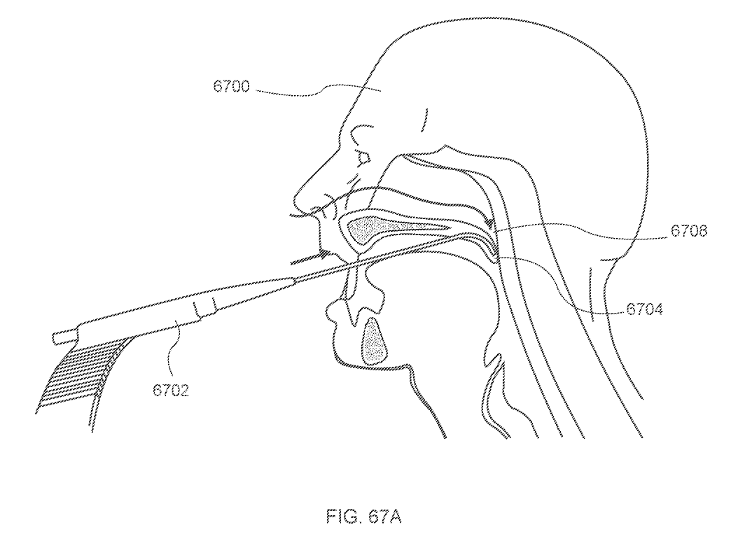

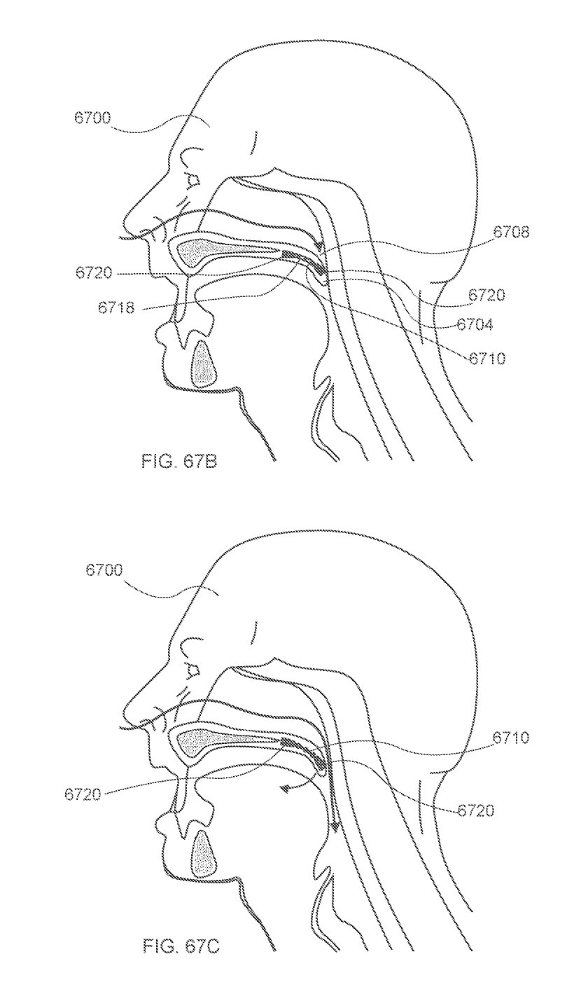

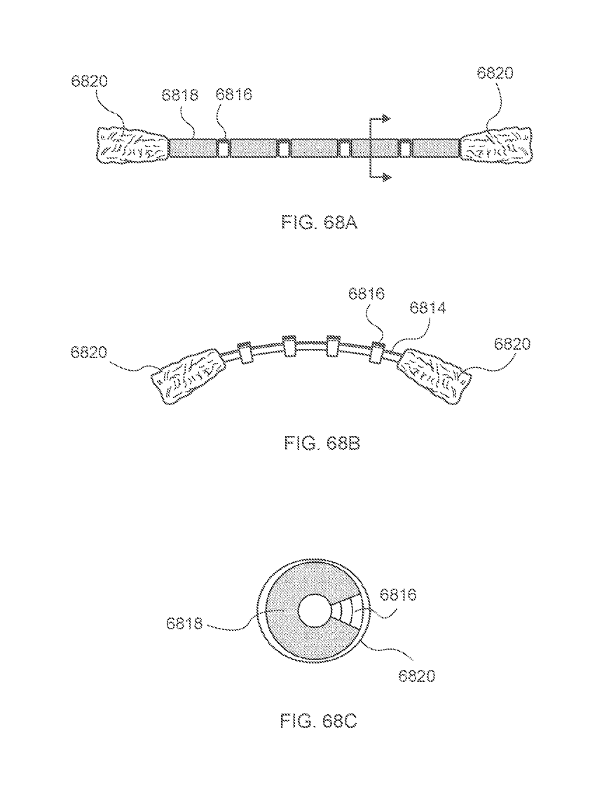

In FIGS. 67A-C, a delivery tool 6702 has been inserted intraorally into the patient 6700 to deliver an airway maintaining device 6710 into a region of the patient's soft palate 6704 forming part of the patient's airway 6708, which is shown as being blocked in FIG. 67A. Device 6710 is described in further detail below with respect to FIGS. 68 and 69. As shown in FIGS. 67B and 69A, the device 6710 is delivered in an elongated and straightened deformed state. In some embodiments, device 6710 when first delivered is not affixed to the soft palate tissue. Over time, however, tissue may grow into the anchors 6720 of device 6710 and/or other parts of the device. Also over time, bioerodable portions 6718 of device 6710 will bioerode, thereby permitting device 6710 to move toward a shorter and more curved at-rest shape, thereby applying a force to the patient's soft palate tissue to maintain the patency of the airway, as shown in FIGS. 67C and 69B.

FIGS. 68A-C and 69A-B show more details of an airway-maintaining device 6710 suitable for implantation in the soft palate. The device's deformed shape is shown in FIGS. 68A and 69A. In this shape, spacers 6718 formed from a bioerodable material are disposed in narrow regions 6714 of body 6712 between wide regions 6714 of body 6712. Body 6712 is formed from a resiliently deformable material (such as, e.g., silicone rubber, polyurethanes or other resiliently deformable polymer or a coil of stainless steel, spring steel, or superelastic nickel-titanium alloy or other resiliently deformable metal, or a composite of the resiliently deformable polymer and metal) and is deformed into the straight and elongated form shown in FIGS. 68A and 69A. The shorter and more curved at-rest shape of body 6712 is shown in FIG. 68B. This is the shape the device will attempt to return to after the bioerodable portions 6716 bioerode, thereby exerting force on the airway-forming tissue of the soft palate, as shown in FIG. 69B. In this embodiment, anchors 6720 are formed from a non-woven fabric (such as polypropylene or polyester) to promote tissue ingrowth. Other anchors may be used, as desired. In this embodiment, the spacers 6718 are injection molded from polycaprolactone, polylactic acid, polyglycolic acid, polylactide coglycolide, polyglactin, poly-L-lactide and have a C shape, although other manufacturing techniques (e.g., dipping processes for applying the spacers over the resiliently deformable polymer or metal), materials, and other shapes may be used as desired.

FIGS. 70A-B show another embodiment of an airway maintaining device 7000 implanted submandibularly into tongue tissue 7001 forming part of the patient's airway. Device 7000 has anchors 7002 and 7004 which differ from each other. Anchor 7004 is an expandable anchor, such as the self-expandable anchor 6449 described above with respect to FIG. 64I, whereas anchor 7002 is not expandable. As shown in FIG. 70A, device 7000 when implanted into tissue 7001 is in an elongated deformed shape. Over time, bioerodable portions 7006 of device 7000 will bioerode, and device 7000 will attempt to return to its shorter at-rest shape, thereby exerting a force on tissue 7001 to maintain the patency of airway 7008, as shown in FIG. 70B.

FIG. 71 is a graph comparing theoretical average tensile force provided to patient airway-forming tissue by various implantable obstructive sleep apnea therapy devices respect to the amount of stretching experienced by the implant. Tether devices are shown by the two lines formed by the square data points. As can be seen, such rigid devices provide no tensile force on the patient's tissue until all slack has been removed, at which point the tether provides a nearly infinite force, possibly exceeding the patient's tolerance limit.

The curve formed by the round data points show theoretical tensile force applied by magnet-based obstructive sleep apnea implants. As can be seen, such devices have a very narrow operational range falling with the therapeutic range providing a benefit to the patient through the application of a minimum therapeutic force.

The curves formed by the diamond and cross data points show theoretical tensile forces applied by two airway-maintaining devices according to this invention having two different spring constants in their deformable device bodies. As shown, these devices can be designed so that they provide beneficial airway maintenance therapy to the patient over a wide range of lengths.

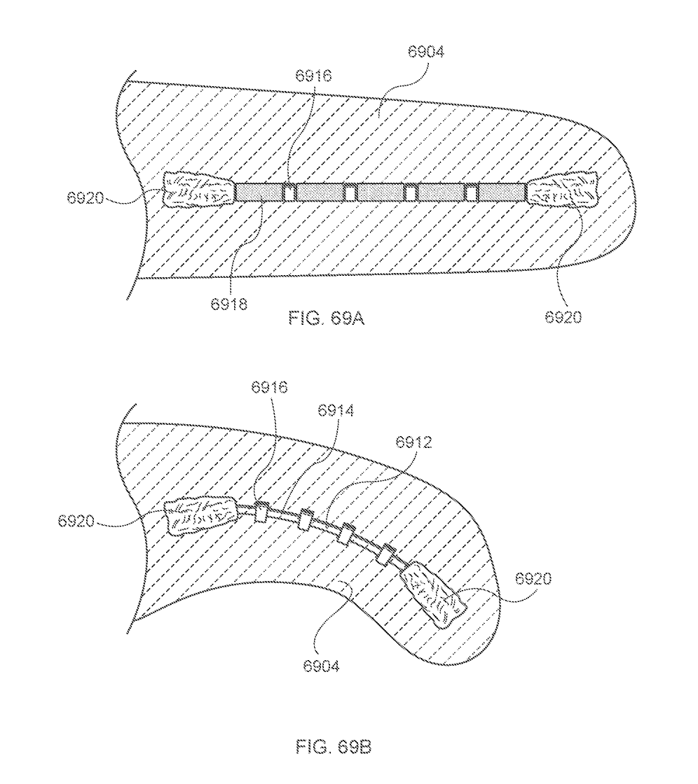

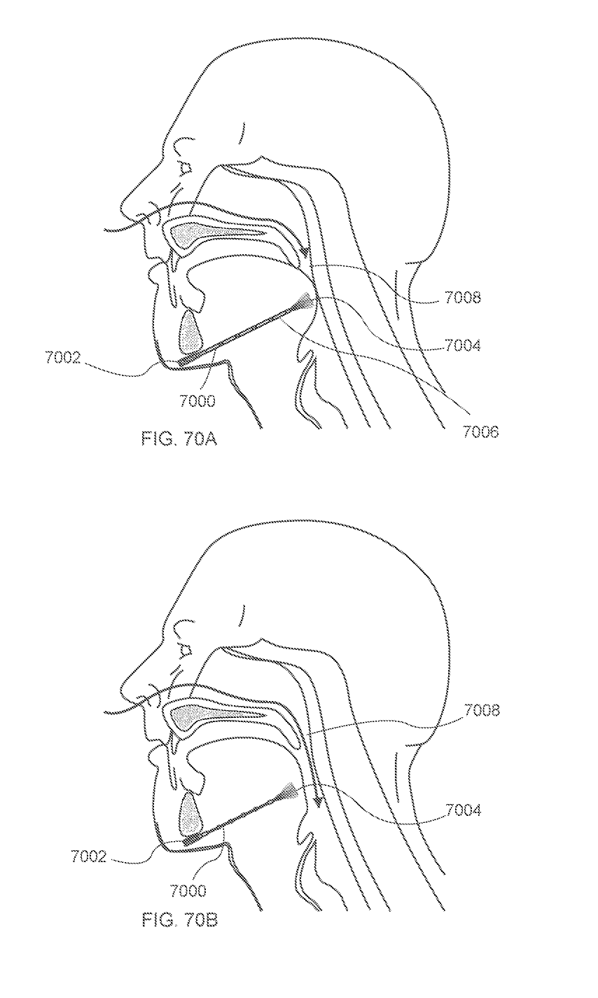

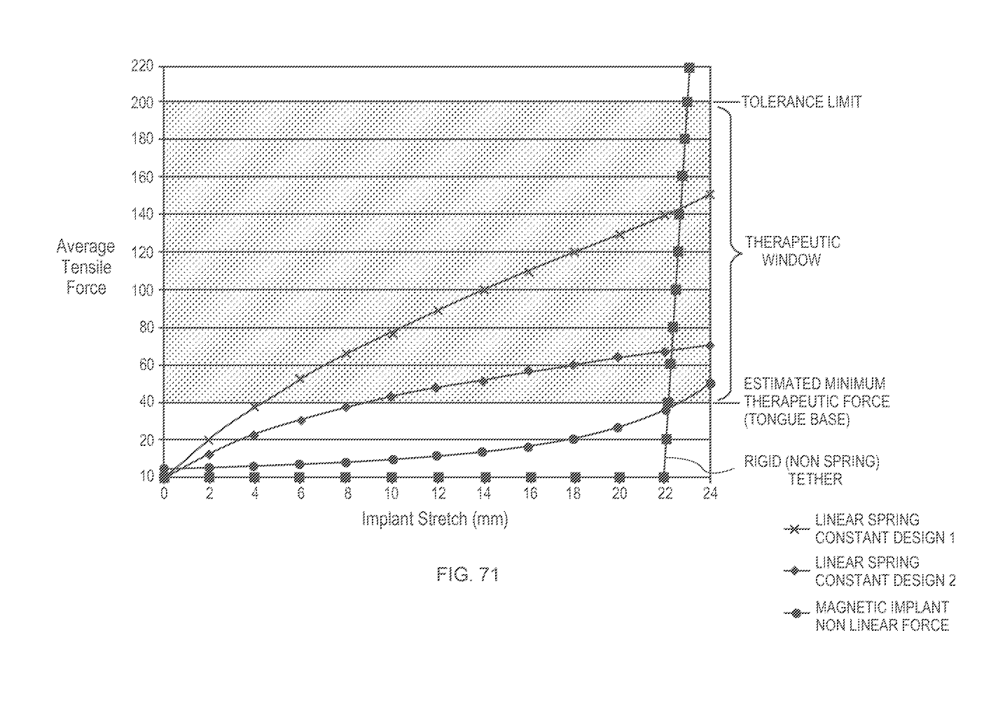

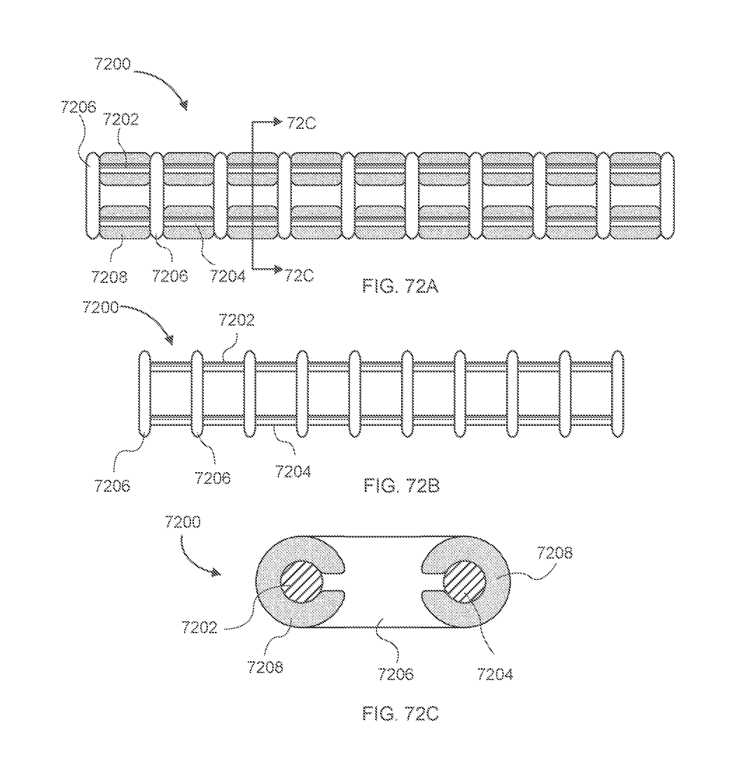

FIGS. 72A-C and 73A-B show yet another embodiment of the invention. Device 7200 has a device body with two elongate rails 7202 and 7204 formed from a resiliently deformable material, such as silicone rubber. A plurality of spaced-apart oval flanges 7206 are attached to rails 7202 and 7204. In the deformed state shown in FIGS. 72A and 73A, C-shaped bioerodable spacers 7208 are disposed between adjacent flanges 7206 to maintain the device in its elongated shape. When spacers 7208 bioerode over time, device 7200 moves toward the at-rest shape shown in FIG. 72B, thereby exerting a force on the patient's airway forming tissue (shown as the tongue 7210 in FIG. 73) to maintain patency of the airway 7212 as shown in FIG. 73B.

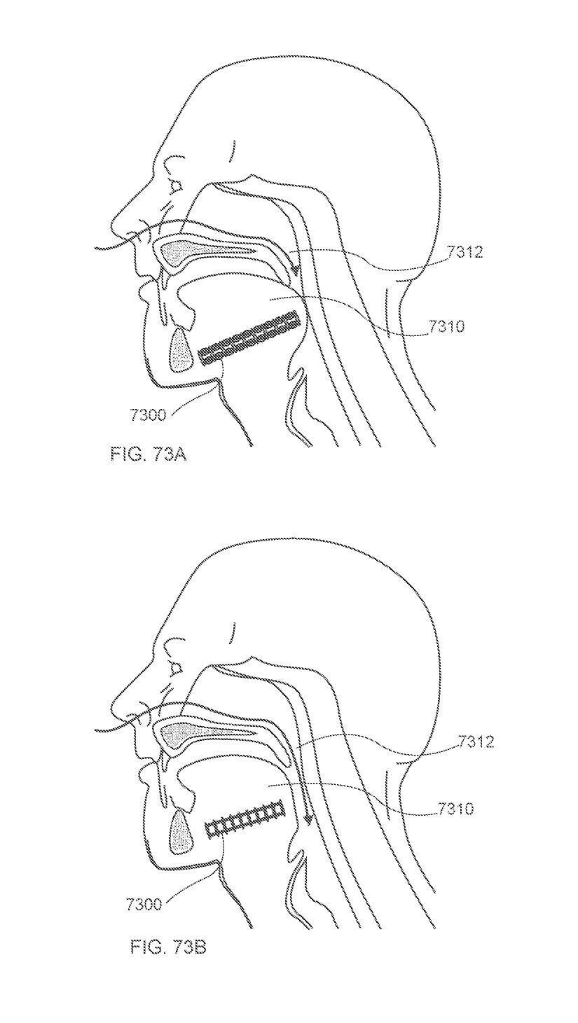

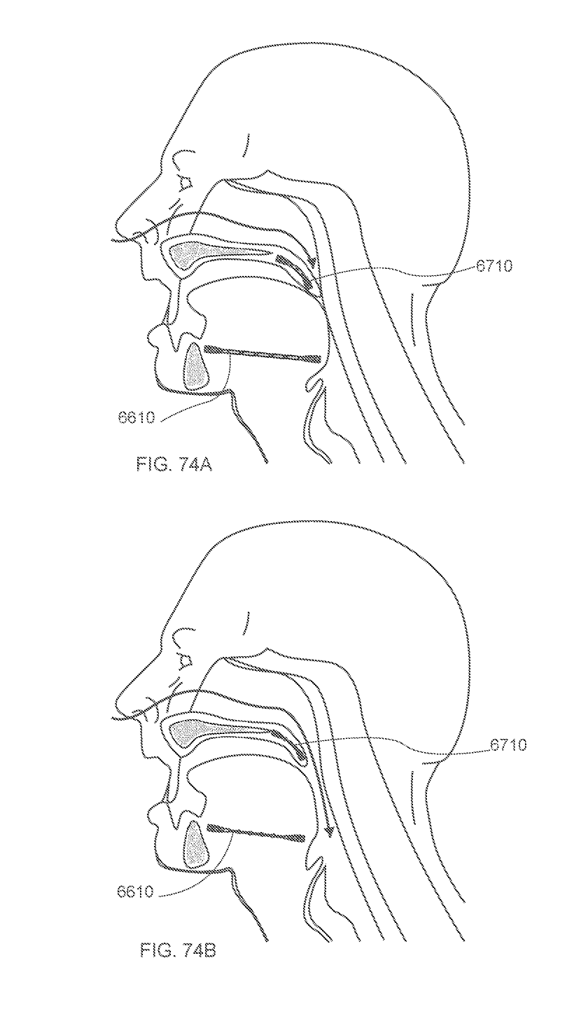

FIGS. 74A-B demonstrate how multiple airway-maintaining devices may be implanted into a single patient, such as the tongue device 6610 and the soft palate device 6710 described with respect to FIGS. 66 and 67 above, respectively.

Likewise, FIGS. 75A-C show how multiple airway-maintaining devices may be implanted into the same region of airway-forming tissue.

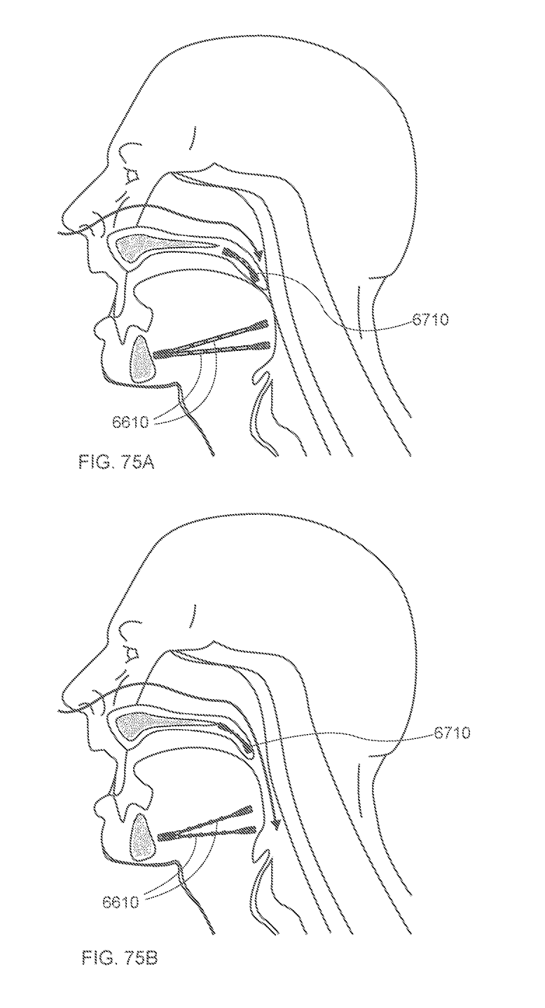

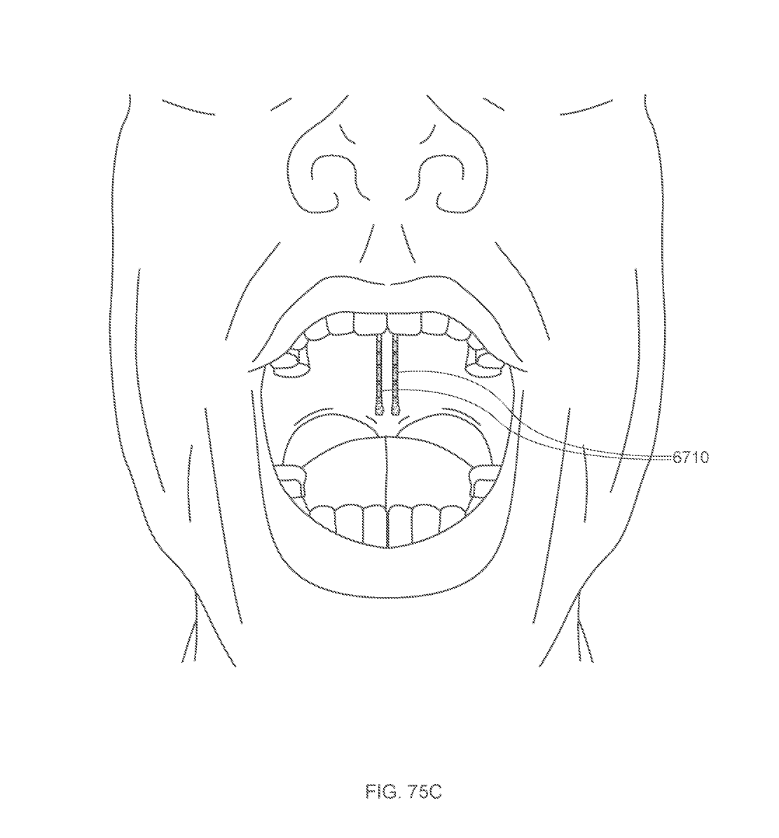

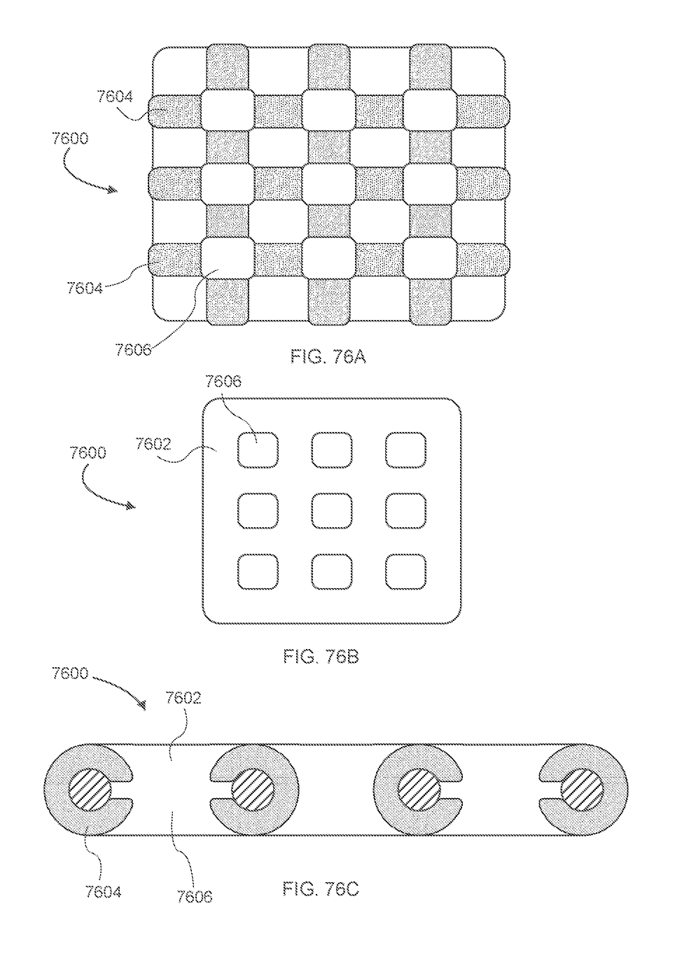

FIGS. 76 A-C show an embodiment of an airway-maintaining device 7600 in which the deformed state of the device body 7602 shown in FIG. 76A is both longer and wider than the at-rest state of the device body 7602 shown in FIG. 76B. Bioerodable spacers 7602 are disposed in openings 7604 formed in resiliently deformable body 7602. As the spacers erode, the body 7602 will move toward its at-rest shape. The openings in the deformed and at rest shapes 7604 and 7606 constitute anchoring elements. This embodiment could be placed in an anatomical structure such as the soft palate and could exert force on the airway forming tissue in two directions to maintain patency.

In some embodiments, the device may include one or more bioactive agents in the bioerodable portion(s). Bioactive agents such as drugs or hormones that are eluted during the course of erosion of the bioerodable materials, may serve, for example, to promote healing of the implant wound, or to promote stabilization of the implanted device within the tissue site by, for example, promoting the toughening the fibrotic tissue capsule that forms around the implanted device.

FIGS. 77A and 77B are schematic views of another embodiment of an in-situ adjustable implant that allows for adjustment of applied force. In FIG. 77A, an elastomeric implant body 7700 has first and second end portions 7705A and 7705B with a medial portion 7710 that can be temporarily maintained in an extended or stretched non-repose position by at least one bioerodible or biodissolvable element or segment, for example segments indicated at 7712a-7712d as described in co-pending application Ser. No. 11/969,201. The medial portion of the implant further comprises a cylindrical reservoir or chamber 7715 enclosed within walls 7718 that can carry a liquid, gel or gas media 7720 that can be increased in volume or decreased in volume to alter the effective length of L of the implant medial portion 7710 after the portions 7705A and 7705B have been secured in the tissue site. In one embodiment, the reservoir 7715 has exterior walls 7718 fabricated of an elastomeric material and configured with a helically woven material or helical spring 7724 that allows for the walls 7718 to stretch and contract axially without substantial change in the cross section of the reservoir within the walls 7718. FIG. 77B shows the implant medial portion 7710 of implant 7700 with altered length L′. In one aspect of a method of the invention, as depicted in FIG. 77B, the in-situ implant can be accessed with a needle 7730 tip that can penetrate the elastomeric wall 7718. The implant can carry at least one marker 7732 such as radiopaque marker(s) to allow the physician to insert to needle precisely into the reservoir. The material of the elastomeric wall 7718, such as silicone (e.g. materials as described in U.S. patent application Ser. No. 11/969,201) has a thickness and modulus that provides for self-sealing after the needle tip 7730 is withdrawn. In one embodiment, the liquid media 7720 can comprise a biocompatible silicone oil or saline solution. In another embodiment of FIG. 77C, the reservoir 7715 can extend over any part of medial portion 7710 such as over the entire length of the medial portion 7710, with a port indicated at 7732. The wall 7718 of the implant body is configured for axial stretching upon pressurizing the chamber 7715 and configured for resisting radial expansion under such pressure. In another embodiment, the reservoir 7715 can be enclosed in a bellows-like structure (not shown). In another embodiment, a gas may be used such as CO₂, nitrogen, argon or another biocompatible gas. It thus can be understood that increasing the effective length L of the implant can reduce forces applied by the implant. Alternatively, decreasing the effective length of the implant can increase forces applied by the implant.

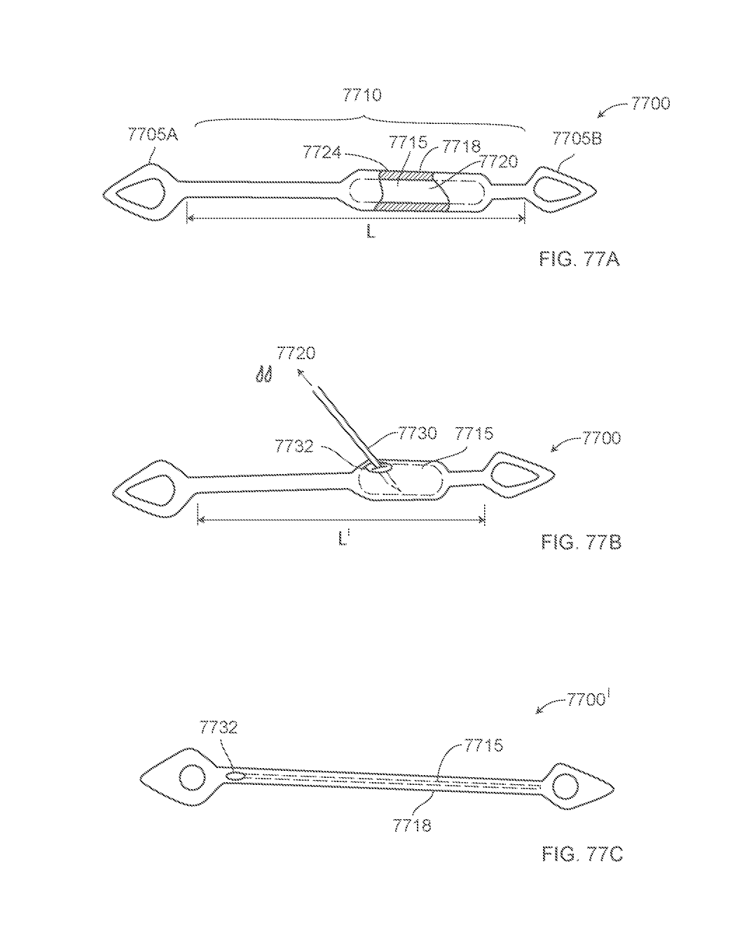

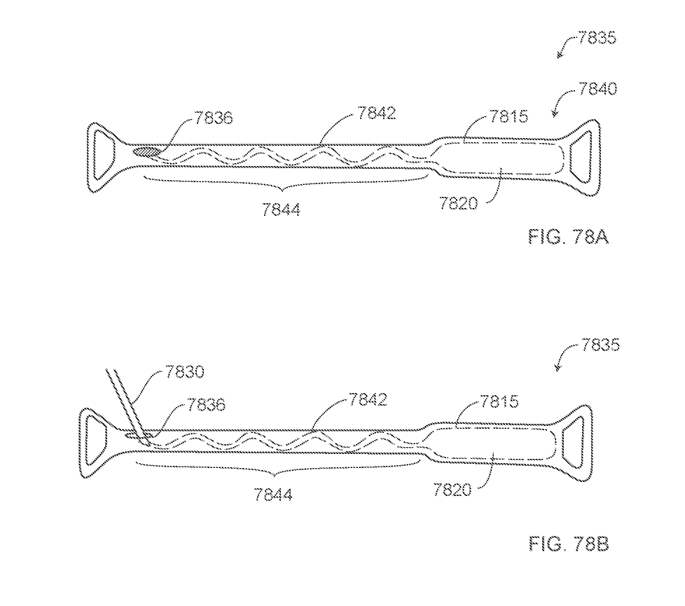

FIGS. 78A and 78B depict an alternative embodiment 7835 wherein the targeted needle port region 7836 adapted for access with a needle is remote from the fluid reservoir or chamber 7815, for example in an opposing axially-extending region 7840 of the implant. The needle port region 7836 is in fluid communication with chamber 7815 via lumen 7842 extending through region 7844. The configuration of FIG. 78A is suited for treatment sites wherein one end of the implant is more accessible to a needle tip 7830. As can be seen in FIGS. 78A-78B, the reservoir or chamber 7815 comprises a lumen portion in region 7840 of the implant which in a first condition is free of a fluid thus allowing the region to apply forces based on the elastomeric material of the implant. To adjust the forces applied by the implant, an incompressible fluid 7820 can be injected into the implant which will occupy the chamber 7815 thus preventing the elastomeric material of the implant in region 7840 from applying forces to tissue, at the same time as allowing the remainder of the elastomeric material to apply forces to the treatment site. It can be appreciated that the implant may be implanted with the chamber 7815 in region 7840 filled with a fluid, and the adjustment comprises utilizing the needle tip 7830 to extract fluid from the implant or add additional fluid to the implant. As can be seen in FIG. 78A, to insure that the incompressible fluid 7820 in region 7844 does not impinge significantly on the function of the elastomeric in said portion 7844, the lumen 7842 is non-axial or non-linear with respect to the implant 7835, but rather is helical, convoluted, zigzag or the like which would still allow the elastomeric portion to function without having to apply forces directly on an axially-extending chamber filled with an incompressible fluid.

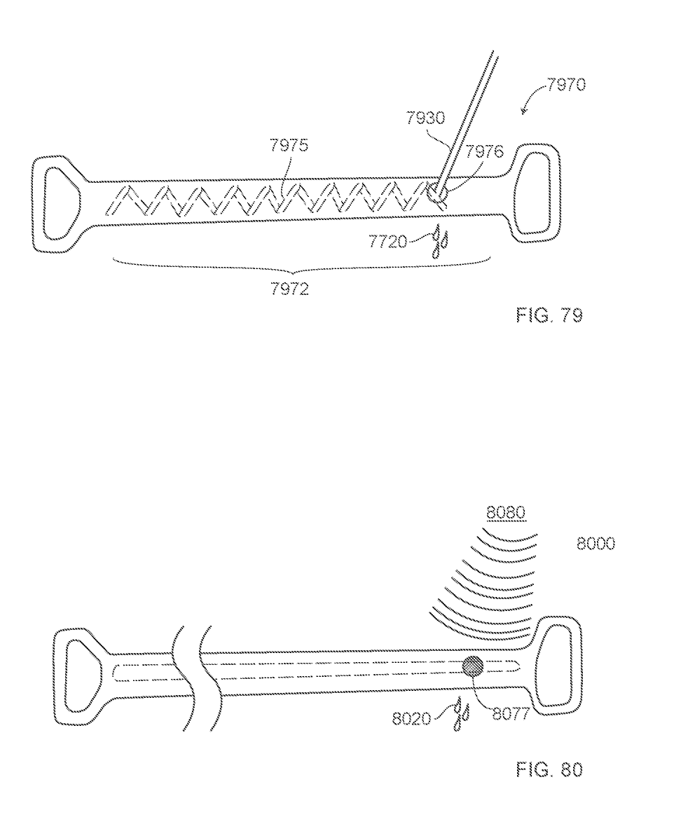

FIG. 79 depicts an alternative embodiment 7970 of in-situ adjustable implant body having an elastomeric medial region 7972 for applying forces to tissue. The medial region 7922 again includes at least one interior chamber 7975 filled with a fluid, for example a biocompatible fluid such as saline 7920, that is filled under pressure with the implant body in a stretched condition. In this embodiment, the chamber 7975 comprises a non-linear lumen, such as a helical lumen, that can be filled with an incompressible fluid or the fluid can be released from the lumen. It can be understood that if the helical lumen is fluid-filled, the elastomeric material can still apply retraction forces after being disposed in a treatment site, but the fluid 7920 will lessen or dampen the applied forces provided by the implant. If the fluid 7920 is evacuated from the lumen 7975, then the elastomeric portion will apply retraction forces without being impinged by the fluid. FIG. 79 depicts a needle tip 7930 puncturing a port region 7976 overlying the fluid chamber 7975 which thus allows the biocompatible fluid to escape into the treatment site. Alternatively, the fluid can be extracted through the needle tip 7930. A similar implant body can be configured with an elongated fluid-filled linear lumen that would restrict movement of the elastomeric body around the linear lumen as in the implant of FIG. 77C.

FIG. 80 illustrates another similar embodiment 8000 except that the implant includes a sacrificial seal or port 8077 that can be sacrificed or dissolved by application of energy from a remote energy source 8080 so that a tool does not need to be penetrated into the treatment site. In one embodiment, an electrical source 8080 can form an electric field and can inductively heat a conductively doped polymer that comprises the seal 8077 to melt the seal and thus release the biocompatible fluid. In another embodiment, light energy that produces a wavelength sufficient to heat a sacrificial seal may be used, or a coil may be provided in the implant that is responsive to electrical energy to create a current in the implant to sacrifice the seal 8077.

FIG. 81 depicts another similar embodiment 8180 wherein the implant carries a plurality of non-linear lumens 8182A and 8182B that each are filled with an incompressible fluid 720 that can be released independently through a seal 8185A or 8185B such as by any means described above to adjust the retraction forces applied by the implant. In the implant of FIG. 81, two helically-configured lumens 8182A and 8182B that overlap are shown, but the plurality of lumens can range from 2 to 10 or more and comprise axially overlapping lumens, partly overlapping lumens or non-overlapping lumens.

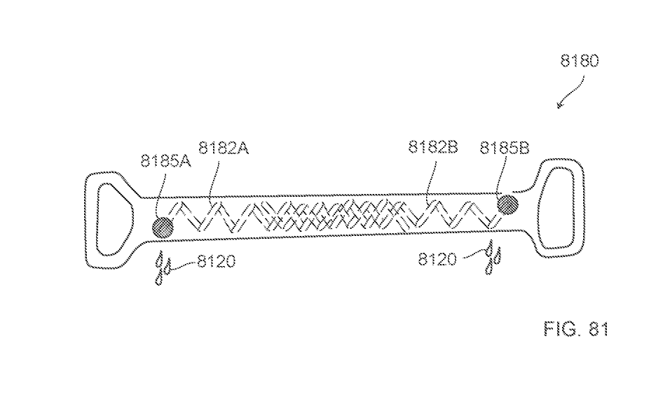

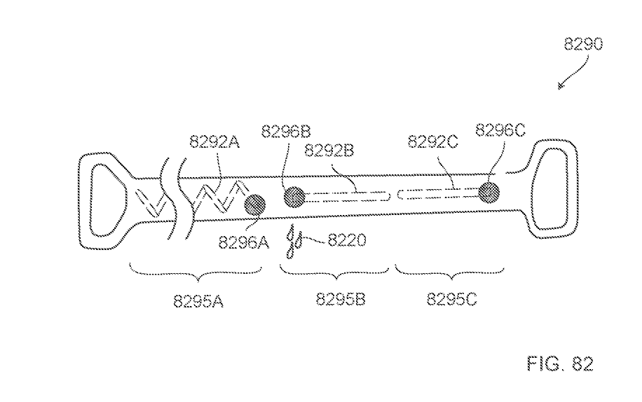

FIG. 82 depicts an implant embodiment 8290 similar to that of FIG. 81 wherein the implant 8290 again carries a plurality of lumens 8292A-8292C that are both non-linear (helical) and linear—each within elastomeric, axial-extending regions 8295A-8295C, respectively. In this embodiment, it can be understood that each linear lumen 8292B, 8292C is filled with an incompressible fluid 8220 that maintains the associate discrete region 8295B, 8295C in a stretched condition when the implant 8290 resides in a treatment site. Thus, the fluid 8220 in each region is adapted to prevent said regions 8295B, 8295C from applying retraction forces to tissue until the time that a sacrificial port or seal 8296B or 8296C is opened to allow one or more lumens to be freed of fluid 8220. The seals or ports can be opened, for example by any means described above, to thus adjust the retraction forces applied by the implant.

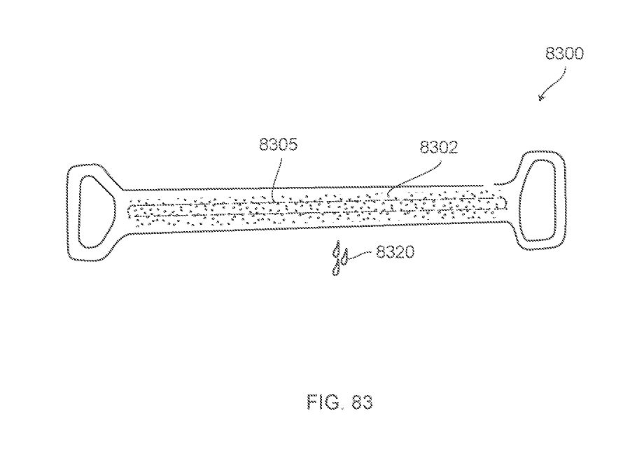

FIG. 83 depicts an alternative embodiment 8300 that is similar to those described above except a permeable wall 8302 surrounding the fluid-filled interior chamber 8305 can be slightly permeable to allow a controlled migration of fluid 8320 from the chamber to thus allow the elastomeric material to apply greater retraction forces to the tissue. The interior chamber or chambers can be non-linear or linear to thus function as described previously to permit the implant to increase retraction forces applied by implant to the treatment site.

In another embodiment, an implant similar to that of FIG. 83 can have an interior chamber filled with a salt and moisture absorbed through the slightly permeable wall can cause the salt to dissolve which will change the forces applied by the implant, typically to reduce the forces applied by the implant.

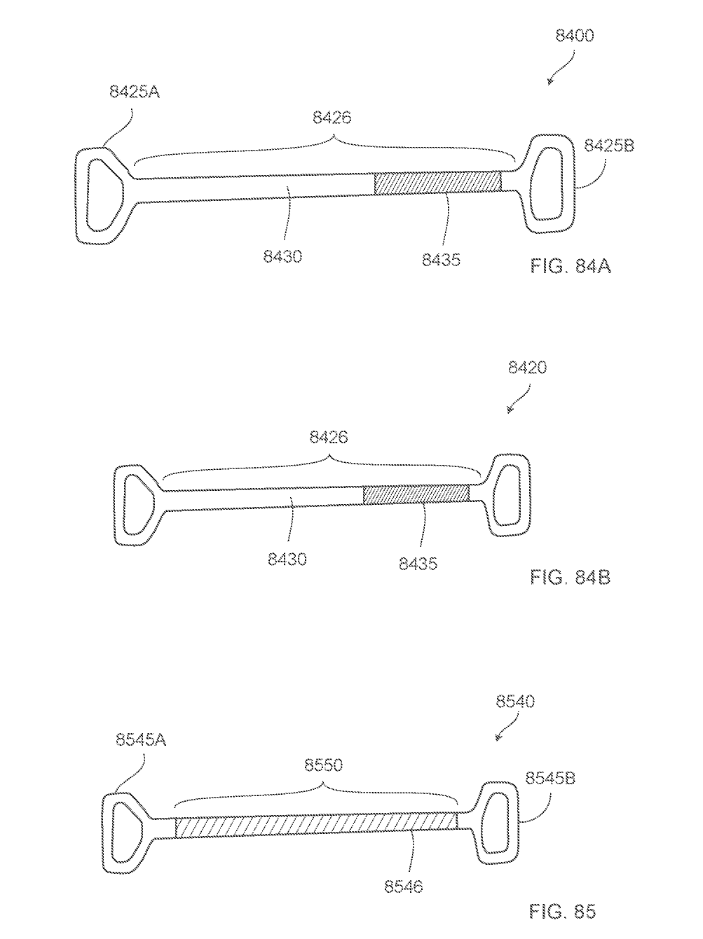

FIGS. 84A-84B depict another implant embodiment 8420 that has first and second end portions 8425A and 8425B with openings therein configured for securing in a treatment site with tissue plugs as describe previously. In this embodiment, the medial portion 8426 of implant 8420 includes an elastomeric portion 8430 that applies retraction forces to tissue as described in previous embodiments. The medial portion 8426 of the implant further includes an adjustable non-elastomeric portion 8435 that comprises a heat-shrink polymer that can be shortened upon heating. In one embodiment, the heat shrink material 8435 can comprise a conductively-doped heat-shrink polymer that can be inductively heated to thereby increase in temperature cause its shrinkage and adjust upwardly the forces applied by the implant to the engaged tissue. FIG. 84B shows the medial portion 8426 of the implant being shortened by actuation of the heat shrink material 8435.

FIG. 85 depicts another implant 8540 with end portions 8545A and 8545B with openings configured for growth of tissue plugs therethrough as described previously. The implant can function in a manner similar to that of FIGS. 84A-84B. In implant 8540 of FIG. 32, the implant has a medial portion 8546 comprising at least in part a shape memory polymer (SMP). By the term shape memory polymer, it is meant that the polymer demonstrates the phenomena of shape memory based on the fabrication of a body comprising a segregated linear block co-polymer, typically of a hard segment and a soft segment. The shape memory polymer generally is characterized as defining phases that result from glass transition temperatures (Tg) in the hard and soft segments or other types of phase change. The hard segment of SMP typically is crystalline with a defined melting point, and the soft segment is typically amorphous, with another defined transition temperature. In some embodiments, these characteristics may be reversed together with the segment's glass transition temperatures. The SMP portion 8550 of the implant body can be fabricated to an initial extended (temporary) memory shape. In such an embodiment, when the SMP material is elevated in temperature above the melting point or glass transition temperature of the hard segment, the material is then formed into its memory shape. The selected shape is memorized by cooling the SMP below the melting point or glass transition temperature of the hard segment. When the shaped SMP is cooled below the melting point or glass transition temperature of the soft segment while the shape is deformed, that temporary shape is fixed. The temporary shape can comprise an extended shape, a non-extended shape or any other shape for implanting in a treatment site.

The original memory shape is recovered by heating the material above the melting point or glass transition temperature T_{g} of the soft segment but below the melting point or glass transition temperature of the hard segment. (Other methods for setting temporary and memory shapes are known which are described in the literature below). The recovery of the original memory shape is thus induced by an increase in temperature, and is termed the thermal shape memory effect of the polymer. The transition temperature can be body temperature or somewhat below 37° C. for a typical embodiment. Alternatively, a higher transition temperature can be selected and a remote source can be used to elevate the temperature and change the SMP structure to its memory shape (i.e., inductive heating or light energy absorption). Referring to FIG. 85, the shape memory polymer portion of the implant can be conductively doped to allow for inductive heating, or an inductively heated material may comprise a jacket around the SMP or be embedded in the SMP. Thus, heating the SMP can cause a change in its length to a greater length or less length.

The SMP component 8550 of the implant of FIG. 85 can also be used to directly adjust another parameter of the implant 8540 to alter applied forces, other than the length of the implant. In other words, the thermal shape memory effect of the polymer can be configured to provide a memorized physical property of the SMP portion which can be controlled by its change in temperature or stress, for example the parameter can comprise the elastic modulus, hardness, flexibility or permeability. Examples of polymers that can be utilized in the hard and soft segments of SMPs include polyurethanes, polynorborenes, styrene-butadiene co-polymers, cross-linked polyethylenes, cross-linked polycyclooctenes, polyethers, polyacrylates, polyamides, polysiloxanes, polyether amides, polyether esters, and urethane-butadiene co-polymers and others identified in the following patents and publications: U.S. Pat. No. 5,145,935 to Hayashi; U.S. Pat. No. 5,506,300 to Ward et al.; U.S. Pat. No. 5,665,822 to Bitler et al.; and U.S. Pat. No. 6,388,043 to Langer et al.; Mather, Strain Recovery in POSS Hybrid Thermoplastics, Polymer 2000, 41(1), 528; Mather et al., Shape Memory and Nanostructure in poly(norbornyl-POSS) Copolymers, Polym. Int. 49, 453-57 (2000); Lui et al., Thermomechanical Characterization of a Tailored Series of Shape Memory Polymers, J. App. Med. Plastics, Fall 2002.

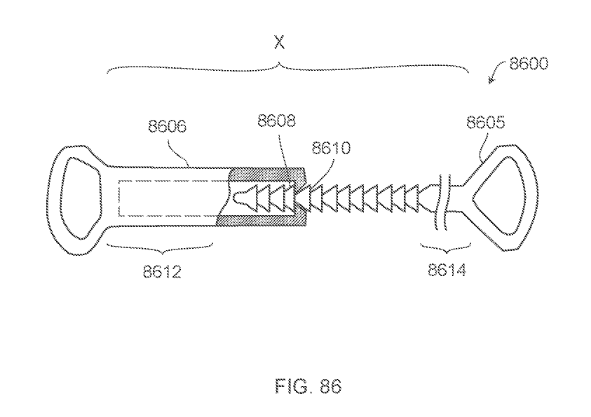

FIG. 86 depicts another embodiment of OSA implant 8600 that is adapted for implantation with a first extended length X and thereafter can be actuated to move the implant toward a second less extended length. In one embodiment and method of the invention, the implant 8600 is implanted in a treatment site such as a patient's tongue. According to the method of adjustment, rather than accessing the implant with a tissue-penetrating tool, the implant 8600 of FIG. 86 is configured to be shortened by physical manipulation of the tongue by gripping the exterior of the tongue with fingers or a suitable jig or device to move a first component 8605 of the implant 8600 relative to a second component 8606 wherein a slightly flexible tooth mechanism 8608 is configured to grip one of a series of tooth-engaging elements 8610. It can be understood that regions 8612 and/or 8614 can comprise an elastomeric portion of the implant, and that the tooth mechanism comprises an independent length adjustment mechanism. The system also can include any latch mechanism or the like that can be manipulated manually to alter the forces applied by the implant.

It should be appreciated that the method of manipulating the exterior of the tongue to actuate a force-receiving mechanism carried by the implant body can be utilized in implants in any airway-interface tissue described above. In another system and method embodiment, the patient can utilize such external manipulation to actuate a fluid-filled implanted squeeze bulb component carried by the implant body, or separated from but communicating with the implant body, to move a fluid into or out of a chamber in an implant body to adjust forces applied by an implant body as described above. The chamber of the implant body can include a leaky valve to slowly allow the biocompatible fluid to return to the bulb over a time interval such as any planned sleep interval. In another embodiment, the system can have first and second squeeze bulbs to allow for manipulation to move the fluid into the chamber in the implant body and the out of the chamber in the implant body, respectively. A system for moving fluid into and out of a chamber of an OSA implant also can be operatively coupled to a pump known in the art for pumping the fluid in a microchannel of the implant, with the pump stimulated by a remote energy source. In this embodiment, the implant thus can be adjusted by the patient following implantation between first and second conditions on a repetitive basis, in one example, for greater applied retraction forces during a sleep interval and for lesser or no applied forces during awake intervals.

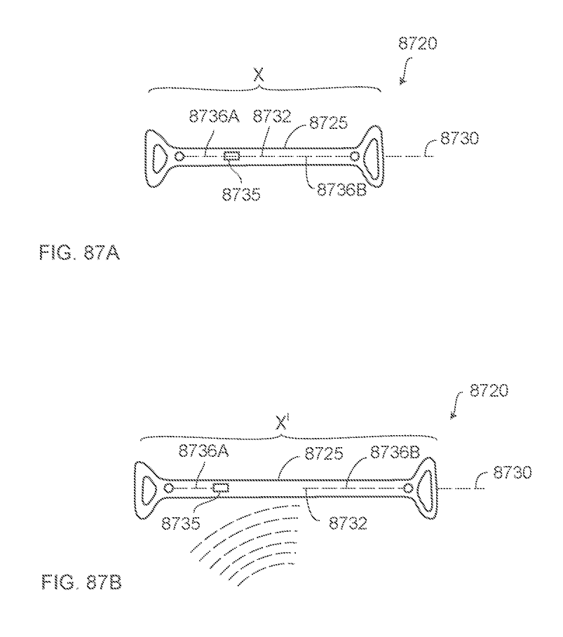

FIGS. 87A and 87B depict another embodiment of OSA implant 8720 that is adapted for implantation with a non-extended length X and thereafter can be actuated to move the implant toward a second extended length X′. In one embodiment and method of the invention, the medial portion 8725 of the implant comprises an elastomeric material that is axially compressed along axis 8730 and releaseably maintained in the axially compressed condition by an elongate tension element 8732 carried by the medial portion. The tension element further carries release means indicted at 8735 which can comprise a sacrificial element of frangible material that releases first end portion 8736A of the tension element 8732 from the second end portion 8736B of element 8732. In one embodiment, the release mechanism comprises a miniature frangibolt which comprises a shape memory alloy sleeve, such as a nickel titanium alloy sleeve, which instantly elongates after reaching a certain temperature. That trigger temperature may be achieved by a heater that is disposed about the sleeve. In this embodiment, the sleeve expands a predetermined amount between surrounding collars upon heating which breaks a wire element. In the embodiment of FIG. 87A-87B, the NiTi sleeve can be heated by an inductively-heated doped polymer that responds to an alternating electric field (FIG. 87B). In another embodiment, the release element can comprise a sacrificial or fuse-like polymer portion that is sacrificial upon a selected voltage passed through such a release element as described in other embodiments above. While the tension member 8732 in FIG. 87A is shown releasably maintaining the implant in an axially-compressed condition, it should be appreciated that such a tension element or compression element with a frangible or sacrificial element can also be use to releasably maintain an elastomeric implant in an axially-extended condition for implantation in a treatment site.

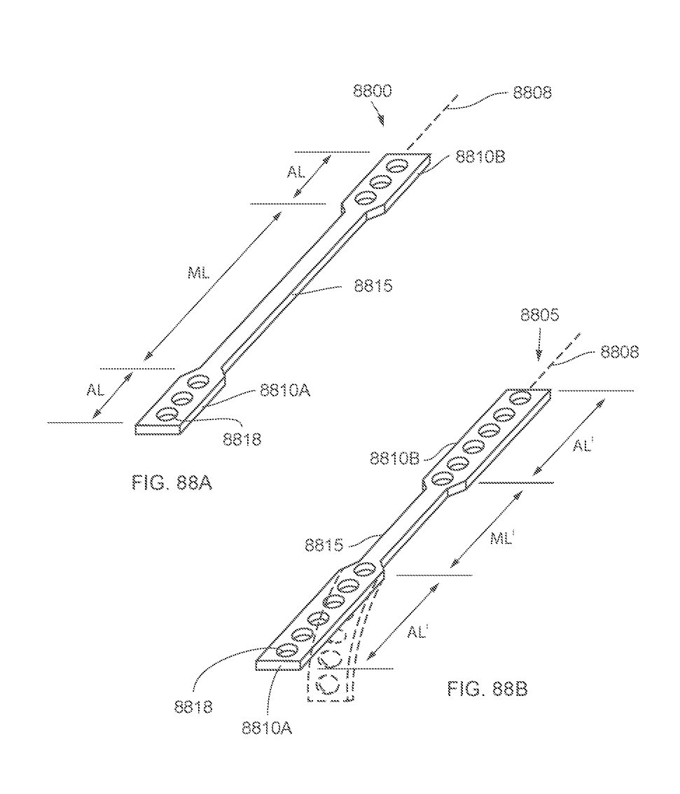

Now turning to FIGS. 88A-88B, other OSA implants 8800 and 8805 are shown. Each implant body include an elastic portion that allows for normal physiological function during non-sleep intervals and can apply sufficient retraction forces along implant axis 1008 to alleviate airway obstruction during sleep intervals. More in particular, the implant shown in FIG. 88A has first and second anchoring end portions 8810a and 8810b that extend about axis 8808 with medial portion 8815 therebetween. The end portions and the medial portion 8815 can comprise a suitable biocompatible elastomer such as silicone. Further, the end portions 1010a and 1010b are configured for anchoring in tissue and thus have openings 8818 or other tissue in-growth features therein as described previously. The medial portion 8815 can be releasably maintained in a stretched configuration during an initial period of tissue in-growth into the end portions 8810a and 8810b as described previously. Of particular interest, the anchoring end portions 8810a and 8810b are flexible but axially non-stretchable or inelastic. The inelastic characteristics of the end portions allow for tissue in-growth to occur more effectively since axial forces are not changing the length of the anchoring end. Further, after the implant is in use to apply retraction forces, each anchoring end portion 8810a, 8810b engages tissue along the entire length of the end portion without greater force being applied to tissue closer to the medial elastic portion 8815, as might otherwise be the case if the anchoring end portion was axially elastic. Elastic anchoring end characteristics, even very small ones, could contribute to unwanted tissue remodeling over time. Therefore, according to some embodiments of the present disclosure, implants are provided having anchoring end portions that do not exhibit even slight axial elasticity.

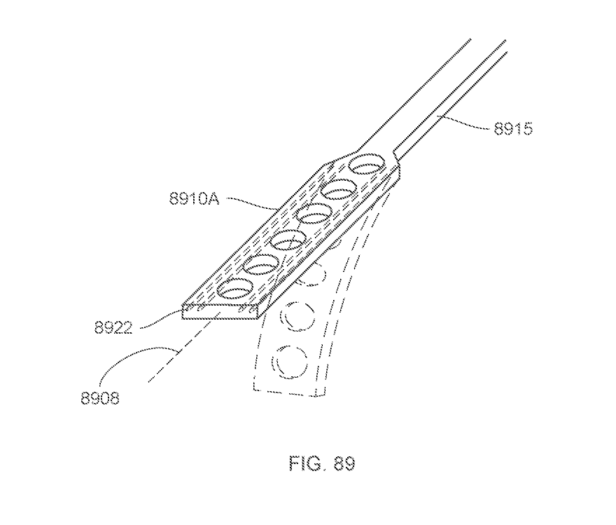

Referring to FIG. 89, it can be seen that an anchoring end portion 8910a of an OSA implant is made axially inelastic by means of non-stretchable reinforcing filaments or elements 8922 embedded therein. Such filaments 8922 can be an inelastic, flexible polymer (e.g., Kevlar®, or polyester), metal wires (e.g. stainless steel, NiTi), carbon fiber or the like. The filaments 8922 can be substantially linear elements or can be knit, woven, non woven, or braided structures as in known in the art. In another embodiment, the anchoring end portion may be made of a non-stretchable material without the addition of reinforcing filaments or elements. As can be understood from FIG. 89, the end portion 8910a is thus axially inelastic but is still flexible and twistable relative to axis 8908.

FIGS. 88A and 88B further illustrate that the anchor portion's axial length of AL (or AL′) can have a selected relationship to the medial portion's axial length ML (or ML′), and thus the overall implant length which is dependent on the desired amount of axial retraction forces applied by the implant. For example, in FIG. 88A, in one embodiment each anchoring end length AL can be 15% of the overall length of the implant which has a medial portion 8815 configured to apply a retraction force of 3.0 Newtons. FIG. 88B depicts another embodiment wherein each anchoring end length AL′ can be 35% of the overall length of the implant and the medial portion 8815 with length ML′ can still be configured to apply a retraction force of 3.0 Newtons. In this embodiment, the design in FIG. 88B may be preferred because of the increased anchoring length, which would decrease the likelihood of tissue remodeling over time.

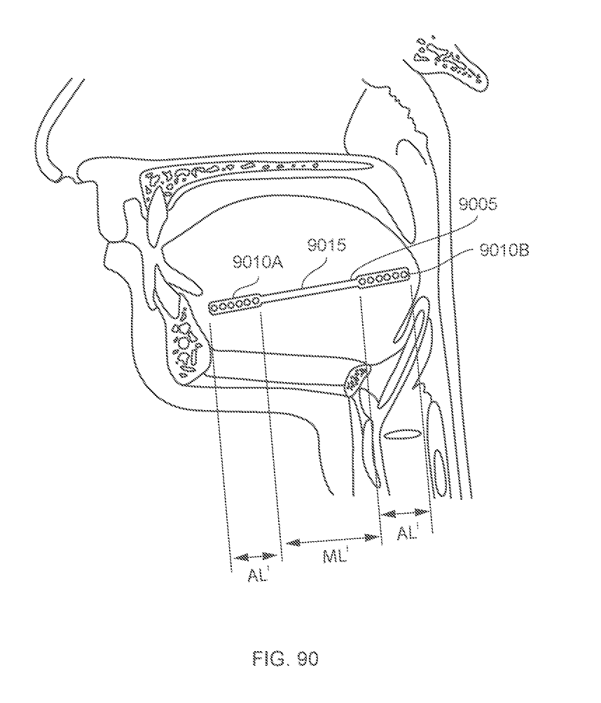

FIG. 90 illustrates a single implant 9005 of the type shown in FIGS. 88A-88B implanted in a patient's tongue wherein each of the anchoring end portions 9010a, 9010b has an axial length AL′ suitable for a particular tissue site, for example close to the base of the tongue and close to the mandible. These end lengths may be the same or may vary, and multiple implants may be used as depicted schematically in FIG. 91. For example, multiple implants in FIG. 91 can collectively apply a selected retraction force and may be used instead of one implant to apply the desired force—but with less force applied per implant 9105, which can reduce remodeling forces applied to any single anchoring end portion.

In general, an implant according to the invention for treating an obstructive airway disorder comprises an elongated implant body having an axis and configured for implanting in airway-interface tissue, wherein the implant body has a medial portion extending between first and second anchoring end portions and wherein the medial portion is axially compliant and the end portions are axially non-compliant. The anchoring end portions are configured for tissue growth therein or therethrough yet allow normal physiological function during non-sleep and sleep intervals. The implant end portions comprise an elastomer with an embedded non-stretchable structure. The implant end portions may have non-isotropic elasticity. The medial portion 1115 can comprise an elastomer or an elastomer with an embedded helical spring element. The medial portion may have isotropic elasticity. The implant can be configured for implantation in the epiglottis, soft palate, pharyngeal wall or tongue tissue.

In one embodiment, the implant has a medial portion extending between the first and second anchoring end portions, wherein each end portion has an axial length of least 15%, 20%, 25%, 30%, 35% or 40% of the overall length of the implant in a repose state of the overall length of the implant. The implant end portions can each have an axial length of at least 4 mm, 6 mm, 8 mm, 10 mm or 12 mm.

In another embodiment, the implant has a medial portion extending between the first and second anchoring end portions, wherein the medial portion has an axial length of least 40%, 50%, 60% or 70% of the overall axial length of the implant.

In some embodiments (not shown), one or more axially non-compliant anchoring end portions may each comprise a single loop of material. The single loop may be provided with an aperture sized and configured to permit tissue to grow therethrough. Non-stretchable reinforcing filament(s) or element(s), such as those depicted in FIG. 89, may extend around the circumference of the loop.

In general, a method for treating an airway disorder comprises implanting an implant in a patient's tongue wherein the implant has first and second end portions that attach to tissue and a tensioned medial portion between the first and second ends, wherein the medial portion is configured to apply a pressure of less than 20 kPa, less than 15 kPa, less than 10 kPa or less than 5 kPa.

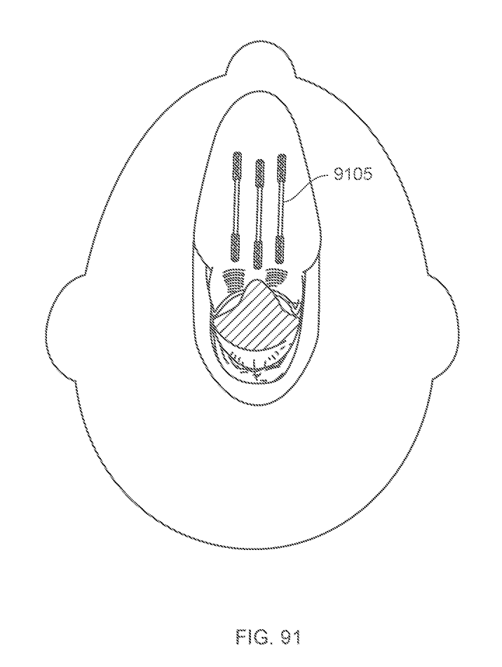

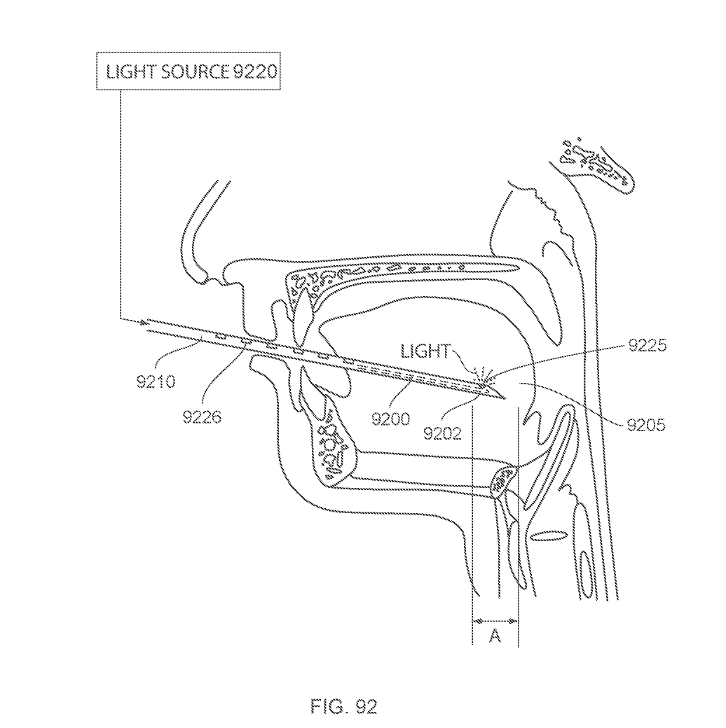

In another aspect of the invention, referring to FIG. 92, another apparatus and method is shown for implanting an implant 9200 and localizing the distal anchoring end 9202 of the implant in the base 9205 of a patient's tongue. In FIG. 92, it can be seen that an elongate, sharp-tipped introducer 9210 carries the implant 9200 in an interior passageway, as described previously. In this embodiment, the system includes a light source 9220 that is coupled to a light emitter 9225 carried at a distal end of the introducer. The light source can be any non-coherent or coherent light in wavelength(s) that will be visible by the physician during the implantation procedure. In use, the physician can observe the light as the introducer penetrates closer to the surface of the tongue, and thus can determine the optimal insertion location of the anchor end 9202 of the implant 9200. In general, it is desirable to position the implant anchor end quite close to the tongue surface, with such a targeted tissue region in the tongue base indicated at A in FIG. 92.

In FIG. 92, it can be further seen that the introducer shaft has markings 9226 along its distal and medial regions (and in some embodiments along the proximal region of the introducer) which can be used to determine the penetration depth when the physician has used the light emission to optimize the location of the distal implant anchor end 9202. The depth of penetration data can be used to load an implant in the interior passageway of the introducer, or can be used to confirm the length of a pre-loaded implant.

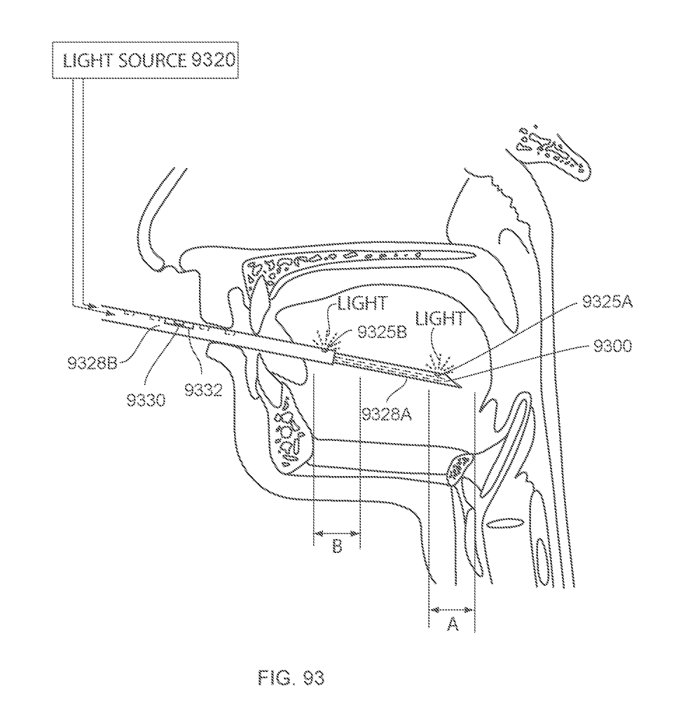

FIG. 93 is a schematic view of another introducer system similar to that of FIG. 92. In this embodiment, the implant 9300 is again carried in a passageway of the elongate, sharp-tipped introducer assembly that includes first and second concentric, slidable sleeves 9328A and 9328B that each carry a light emitter 9325a, 9325b at a distal portion thereof. The emitters 9325a and 9325b are both detachably coupled to light source 9320. It can be understood that the targeted tissue region A in the tongue base can be located with the light emitter as described above. Further, a targeted tissue region B in the anterior portion of the tongue can be located with light emitter 9325b in sleeve 9328B. After both emitters 9325a, 9325b are localized and light emissions are observed, then one of several markings 9330 on inner sleeve 9328A can be viewed through a notch 9332 or window in 9328B to determine the appropriate length of implant 9300. The spacing between the emitters 9325a, 9325b thus can be determined to further determine the appropriate length implant 9300 that can be inserted into an interior passageway in the introducer system. It should be appreciated that visual observation of markings on the introducer sleeves is only one manner of determining the axially spaced apart relationship of the light emitters. The scope of the invention includes other means such as cooperating electrical contacts in slidable sleeves 9328A and 9328B that contact one another to indicate the axial dimension between targeted tissues for anchoring first and second ends of an implant 9300.

FIG. 94 represents another introducer system that functions in a similar manner to the systems of FIGS. 92-93. In this embodiment, the implant 9400 is again disposed in an elongated introducer 9410 that carries a plurality of light emitters 9425a-9425d that are axially spaced apart in a manner that will assist the physician in determining a suitable length of implant, and localizing the anchoring ends of the implant 9400 in tongue tissue. The light emitters 9425a-9425d can range in number from two to ten or more and be spaced apart by a dimension of 1 mm to 10 mm. A controller and switching mechanism may be provided to activate the light emitters one at a time or in sequence. Also, the light emitter can provide different wavelength and thus different visible colors to assist in determining the location of each light emitter in the tissue. Alternatively, the light can be emitted through colored lenses to provide a plurality of colored light emissions.

In general, the term light emitter as used herein includes a remote light source coupled to a light guide in the introducer, wherein the light guide can comprise an optic fiber or other channel with light emission from the distal end of the channel. In the embodiment of FIG. 94, the plurality of emitters can be coupled to a plurality of light guides or a single light guide can have a plurality of light emitting points, for example light emission regions along the length of an optic fiber. In one embodiment, an optic fiber is carried in the wall of the introducer sleeve. In any embodiment, the light emitter also can comprise an LED or similar light emission source disposed on the introducer that is coupled to a power source.

FIG. 95 depicts a method of the invention using an introducer system of FIGS. 92-94 wherein a pusher 9535 is used to stabilize the axial position of the implant while the introducer sleeve 9510 is withdrawn slightly to deploy the distal anchor end 9502 of the implant 9500 in the targeted location. With the anchor end 9502 and openings 9536 exposed in tissue, the physician can further penetrate a second introducer 9538 along path P into and through an opening 9536 to further stabilize the distal anchor end in the tissue. The second introducer can also deploy a second implant (not shown) that forms a cross-bar with implant 9500. The second implant thus can distribute forces over a larger portion of the tongue base.

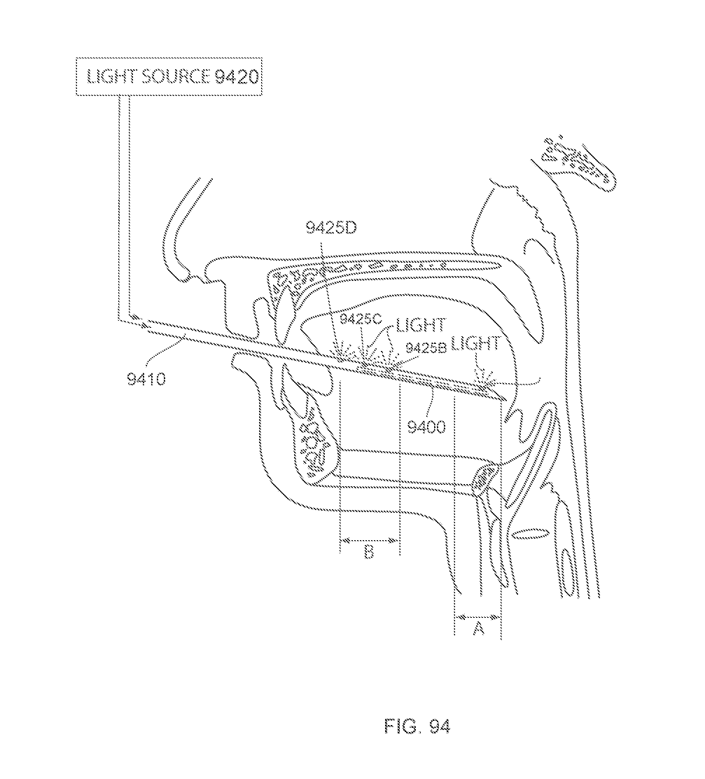

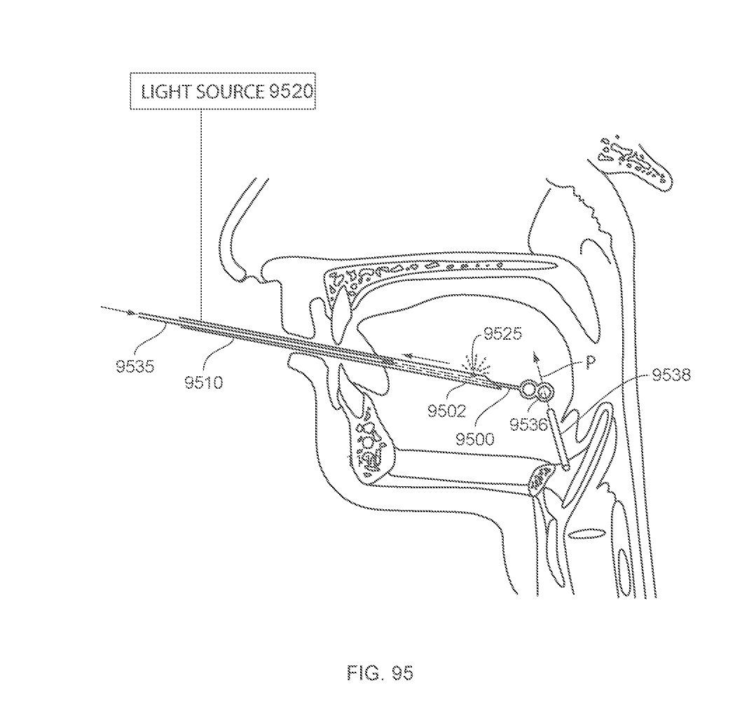

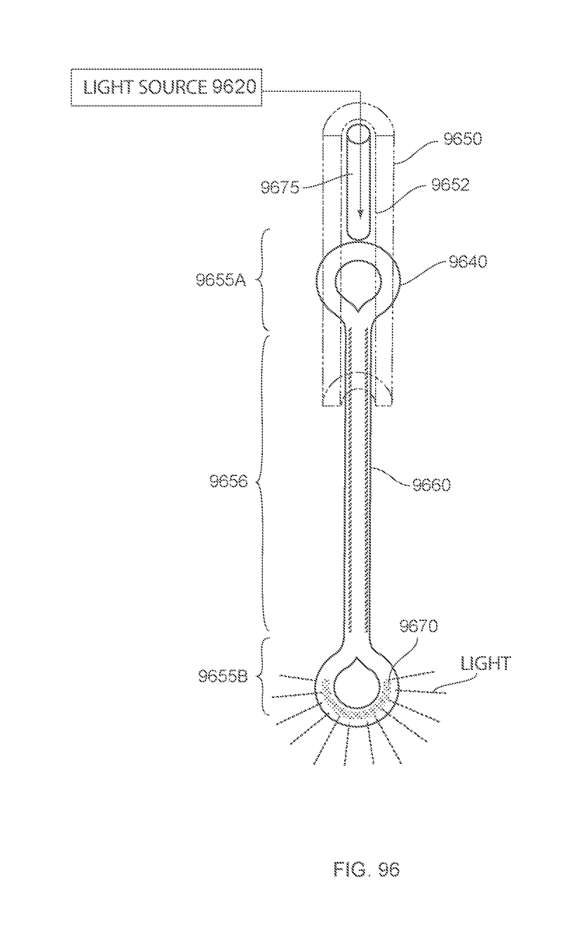

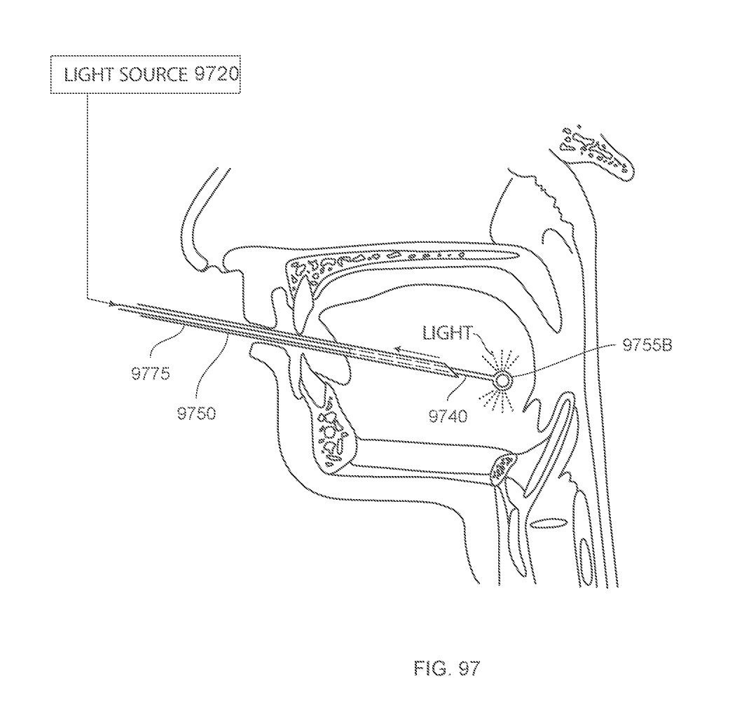

FIGS. 96-97 illustrate another implant 9640 and method corresponding to the invention. In this embodiment, the introducer system includes an introducer sleeve 9650 (distal portion in phantom view) with an interior passageway 9652 for carrying the implant 9640. The implant has a proximal anchor end 9655A and a distal anchor end 9655B. The implant 9640 is configured to function as a light channel and light emitter. More particularly, the implant can be fabricated of a polymer that is transparent or translucent, with the proximal anchor end 9655A free of any reflective material to allow light transmission therethrough. The medial portion 9656 of the implant body carries tubular region of reflective material to provide a light guide region indicated at 9660. Alternatively, a flexible optic fiber may be provided in the implant. The distal anchor end 9655B of the implant carries reflective material 9670 that can reflect light generally to allow viewing of the anchor end when illuminated. Thus, the medial portion 9656 of the implant comprises a light guide that allows light propagation therethrough by internal reflection in the light guide region 9660, and then outward light emission by the reflective material 9670.

In the introducer system of FIG. 96, the light can be delivered by a removable, elongate member 9675 with a light guide therein that is inserted in passageway 9652, or the walls of the passageway 9652 itself may be internally reflective to serve as a light guide. The light guide member 9675 thus also can be used as a pusher and/or puller member to assist is deploying the implant 9640. FIG. 97 shows a method of using the invention wherein the implant 9740 has its distal anchor end 9755B disposed in a targeted tissue region with the introducer sleeve being withdrawn, and light being emitted from the anchor end 9755B of the implant.

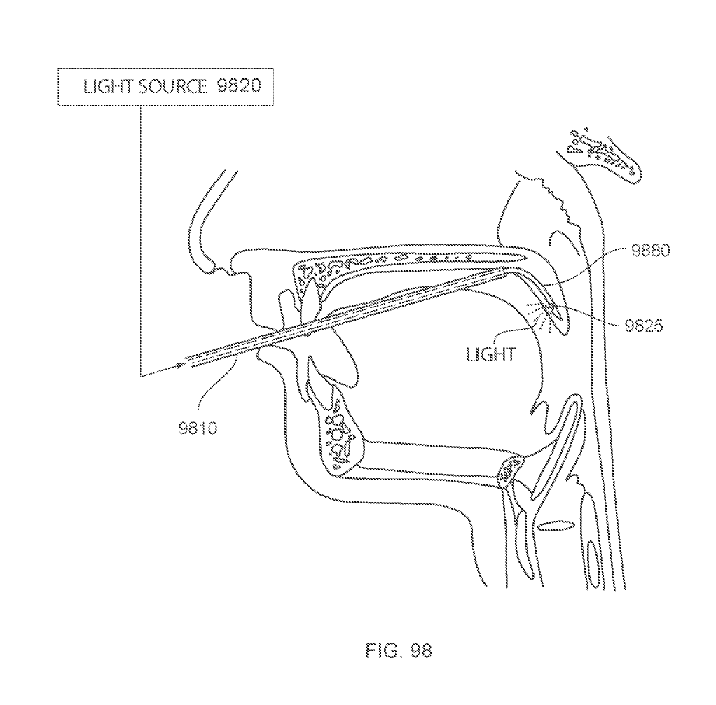

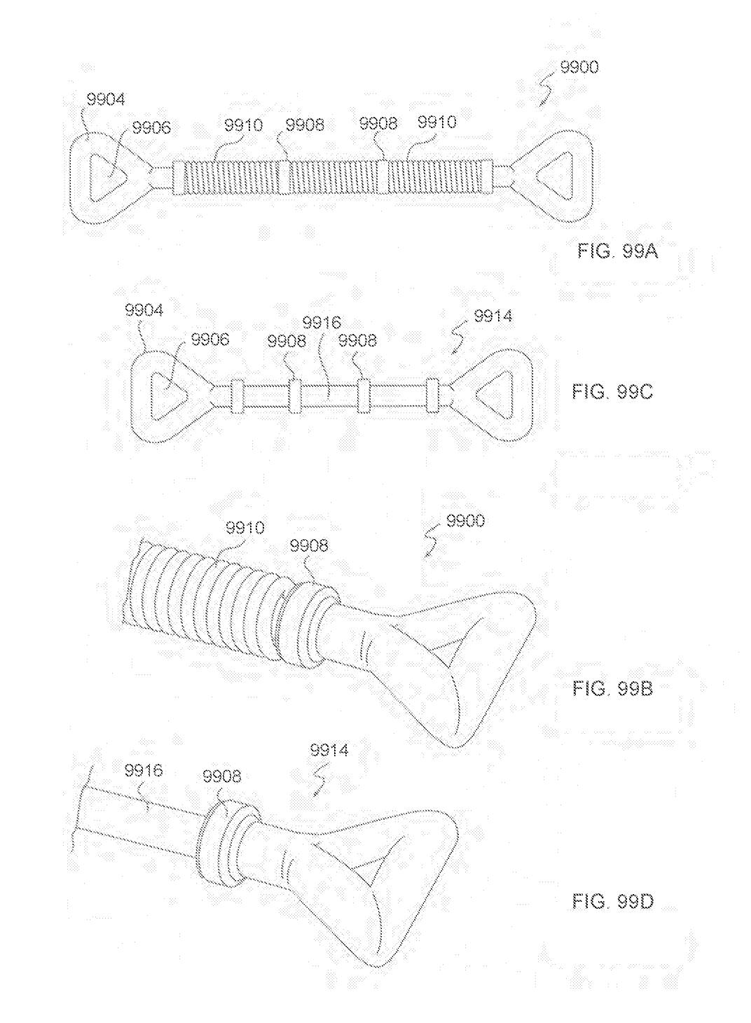

FIG. 98 illustrates another system embodiment configured for deploying an implant in soft palate tissue, wherein the introducer system can have the light emitter 9825 carried by a curvilinear introducer sleeve 9880. In all other respects, the system would generally function as any above described embodiment.

In general, a method of treating an airway disorder according to some aspects of the invention comprises introducing an introducer working end carrying a deployable implant into an airway-interface tissue, and localizing an implant anchoring end within the tissue by observing light emission from an emitter in the working end. The light emission can be provided by light propagating in a light channel extending to the working end, or from an LED carried by the working end.

Another method for treating an airway disorder comprises introducing an introducer working end carrying a deployable implant into an airway-interface tissue, and localizing an anchoring end of the implant in the tissue by observing a light emission from the implant.

In another aspect, an implant according to the invention for treating an obstructive airway disorder comprises an elongate body configured for implanting in an airway-interface tissue wherein at least a portion of the elongate body carries a light guide for directing light transmission therethrough. Further, the implant includes a body portion that carries a light reflective material for reflecting light transmission therein.

FIGS. 99A-D show an embodiment of the invention similar to the devices shown in FIGS. 64A-J with long term elongate implant portion 9914 shown without a bioerodable material portion (FIGS. 99 C-D) and implant system 9900 shown with bioerodable portion 9910 at least partially enveloping the long term elongate implant portion of the device (FIGS. 99 A-B). In this example, the bioerodable portion is helically wound around the elongate implant. Bioerodable portions 9910 are adjacent or connected with wide sections 9908 of the long-term implant and may be configured to resist a compressive force from the long term implant or to apply an expansive force to the long term implant. The long term implant may be made of a resiliently deformable material, such as a silicone material (e.g. silicone rubber), polyurethane or other resiliently deformable polymer or a coil of stainless steel, spring steel, or superelastic nickel-titanium alloy or other resiliently deformable metal, or a composite of the resiliently deformable polymer and metal. In particular, when placed in an animal's body, one or more bioerodable portions 9910 may hold the elongate implant in a first, elongated shape until the implant is anchored to an airway tissue, such as by a fibrotic response or tissue growth through one or more holes 9906 in anchor end(s) 9904. Over time, the bioerodable material bioerodes, the device shortens and exerts a therapeutic force on airway tissue. Compare the relative compositions, shapes, and lengths of foreshortened device 9914 (FIGS. 999C-D), having exposed narrow sections 9916 after bioerosion, with device 9900 (FIGS. 99A-B) before substantial bioerosion has taken place and having bioerodable material 9910 partially enveloping the long-term implant.

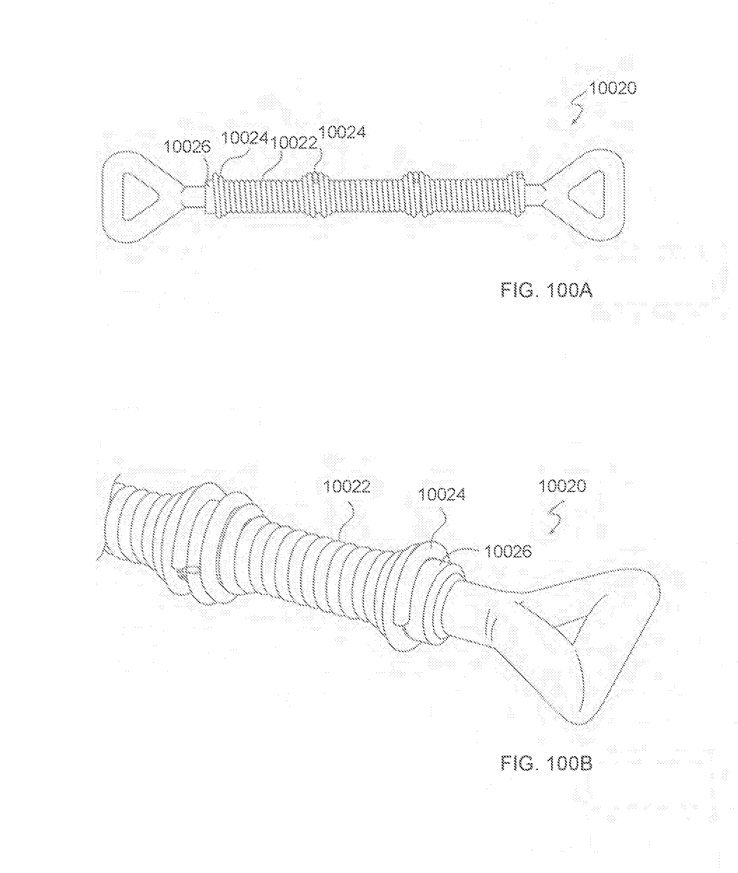

It may be beneficial for a device to remain in an elongated shape until substantial or sufficient tissue growth has taken place and the ends of the device are anchored into airway tissue. However, in some conditions a device might not maintain a sufficiently elongated, (stretched) configuration as shown in FIGS. 99A-B for a period of time sufficient to allow tissue to anchor the implant into tissue (e.g. to anchor the implant so that it can exert a desired therapeutic force on the tissue). Rather, if the implant shortens too much from its first, elongated shape before becoming anchored in tissue (or does not become anchored), it may not be capable of exerting sufficient force on airway tissue to have a therapeutic effect. Some specific devices, similar to those shown in FIGS. 99 A-B, when tested in an animal model, did not exhibit the desired tissue effect. It is hypothesized that normal airway tissue movement imposed mechanical forces on the devices that led to premature device foreshortening. See Example 2. In particular, in vitro testing showed that one explanation for the lack of the desired tissue effect could be premature long-term implant foreshortening due to mechanical agitation causing its premature release from the bioerodable material that otherwise holds it in an elongated configuration (shape). When implant systems were subject to ultrasound vibration in a saline bath (to model or mimic the implant environment in a body), coiled bioerodable material, such as that shown in FIGS. 99A-B, unwound relative to the long-term implant axis. See Example 2. FIGS. 100 A-B show an example of an implant, such as the one depicted in FIGS. 99A-B, in which coils including end coil 10024 at an end of bioerodable helix 10022 on device 10020 have unwound and part of wide section 10026 of the long term implant has retracted inside the end coils. The coils remaining wound around the narrow long term implant portion may not be able to provide a sufficient resistive force to hold the long term implant in a desired, elongated configuration (shape). Therefore, it may sometimes be beneficial to create, reinforce or change a device structure such that it will substantially hold a desired shape in vivo for a sufficient period of time (e.g. in the presence of mechanical or other forces) to allow tissue anchoring of the device to take place. Either a bioerodable or a long term implant portion (or both) may be configured or altered to improve the ability of the long term implant to be placed and to remain in a tensioned shape (e.g. held by the bioerodable portion) until sufficient tissue growth has taken place. An initial tensioned shape or configuration may be any shape or configuration that creates a tension between a bioerodable material and a long-term implant that is different from a final shape or configuration (in which the bioerodable material has bioeroded and no longer exerts a tension on the long term implant). Alternatively, an additional piece (such as a holder or clip) may hold the bioerodable portion or long term implant in a preferred shape or configuration.

In some embodiments, an implant system according to the disclosure includes a resilient elongate implant body having a first insertion shape and a second therapeutic shape and a bioerodable material including at least two coils that at least partially envelop the resilient elongate implant body, wherein the coils are coupled together to form a coupled coil structure. A therapeutic shape of an elongate implant body may be a shape that the body takes after a bioerodable portion bioerodes. A therapeutic shape may be a shape configured to exert a desired force) on a target tissue (e.g. an airway forming tissue).

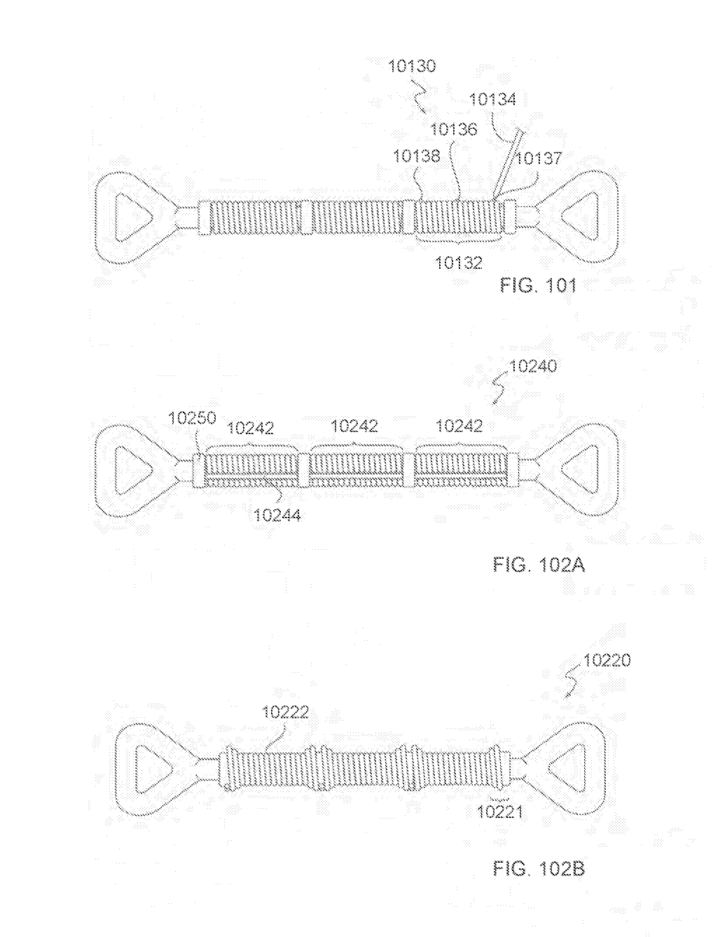

A bioerodable portion may be manufactured to better maintain an initial or desired shape, for example, by changing the way the bioerodable material is otherwise held in place relative to the resilient or elongate long term implant. The bioerodable portion may be held in position along the elongate implant in any way. For example, the portion may be held using a chemical coupling and/or using a mechanical coupling (e.g. an interlocking). To aid in device performance, including maintaining a device shape, portions of the bioerodable implant may be made less flexible compared with other, more flexible sections. The less flexible portions may hold the implant in a first shape and prevent the bioerodable portion from undergoing undesired movement (e.g. unwinding) relative to the long term implant. A point on an implant may be made less flexible, for example, by coupling one portion of a bioerodable material to another portion of the bioerodable material (e.g. coupling to itself). In one embodiment, two points on the bioerodable portion may be fused together. The two points may be on the same coils or may be on different coils. FIG. 101 shows two coils on bioerodable helix 10132 of implant 10130 fused at fusion point or bridge 10137 at a first end to bridge a first region. The bridge may create a region of lesser flexibility on the bioerodable portion, reducing movement of the bioerodable implant, and thereby maintaining the bioerodable material in an enveloping configuration relative to the long term implant. A point connecting a coil and a bridge has lesser flexibility than a flexibility of a (or either) coil to which it is coupled. Any number (or no) bridges may be made on a bioerodable portion. A bridge may have a different flexibility.

In one particular embodiment, a point on a coil may be fused to another point on the same coil.

In some embodiments, coils may be fused at both ends of a bioerodable portion. FIG. 101 shows two coils fused at a first bridge 10137 and a second bridge 10138 at a second end of the bioerodable portion. Fusing or anchoring both ends of a bioerodable portion may prevent a bioerodable implant from rotating or unwinding relative to the long term implant.

In another embodiment, two other coils may be fused to form a third bridge 10136. The third bridge may be anywhere along the bioerodable portion, but in one particular example it is near the middle of a bioerodable portion. The third bridge may, for example, provide additional strength to the bioerodable portion to resist movement caused by mechanical agitation from airway tissue movement and may serve as a backup in the event that one of the first two bridges near an end of the bioerodable portion prematurely breaks while implanted (e.g. breaks before tissue growth has anchored the implant) such that the breakage might otherwise allow the bioerodable portion to unwind and the long term implant portion to prematurely foreshorten. A fourth, fifth, sixth, seventh, eighth or more bridges may be formed. In some embodiments, there may be a bridge at least every 1 mm, every 2 mm, every 3 mm, every 4 mm, every 5 mm, every 10 mm, or every 15 mm. In some embodiments, each coil may be fused to at least one (or at least two) other coil(s). In some embodiments, each coil may be linked to at least one other coil, forming a plurality of bridges. In one particular embodiment, all of the coils are fused together. An implant may have one, two, three or more than three separate bioerodable portions. Any (some, or all) of a bioerodable portion(s) may be fused to itself or one (or more) bioerodable portion(s) may be fused to one or more other bioerodable portion(s).

The bioerodable material may be linked or fused to itself in any way. The points on the material may be fused using a source that can generate energy (heat). The energy (heat) may melt a portion of the bioerodable material to cause it to bind to another portion of the bioerodable material and remain bound after cooling. The heat source may be a direct heat source (such as a soldering iron) that is at a temperature higher than an implant temperature or the heat source may be an indirect source such as a chemical source or light source, vibrational welding, induction welding, ultrasonic welding, or radiofrequency welding source that causes heat to be generated in the implant.

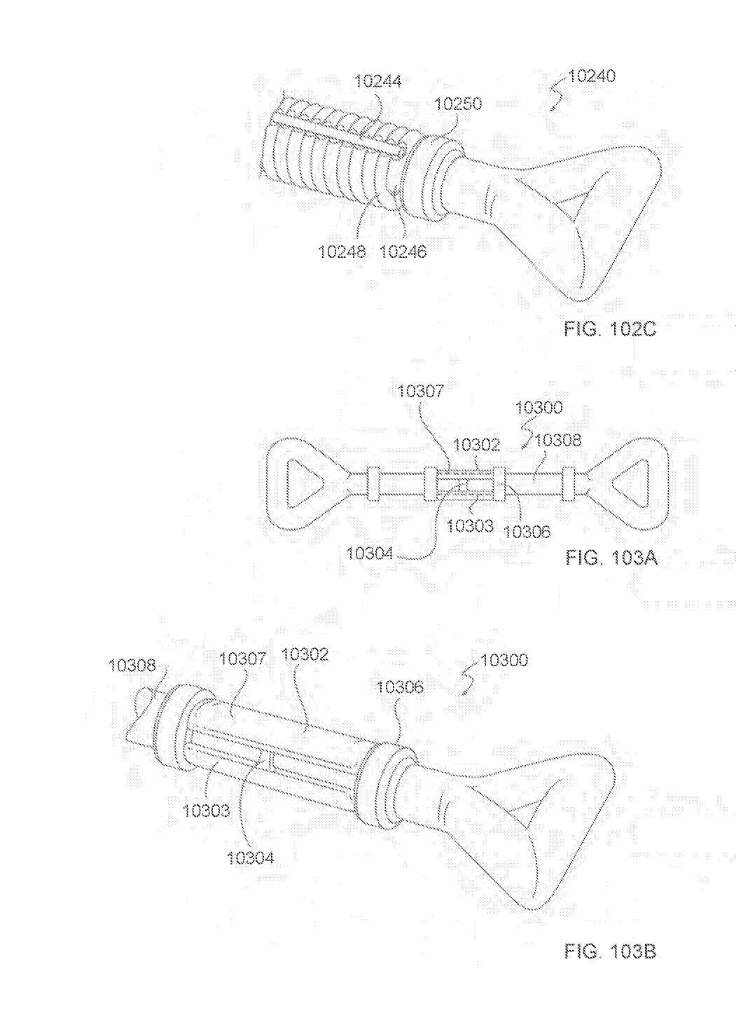

Instead, or in addition to a bioerodable material melting or otherwise linking to itself, a bioerodable material may be coupled to itself using an additional joining material such as an adhesive that bonds or a solvent that bonds by melting adjacent surfaces together or small clip or mechanical attachment to form a bridge and couple two (or more) points on the material. FIG. 102A, C show implant 140 with bioerodable material in the form of helix 10242. Bar (or bridge) 10244 connects essentially all of the coils of a bioerodable portion, creating a support strut that may help hold the helix in an initial shape. The support strut may create a point(s) or region(s) of reduced flexibility on the helix and the helix may then resist movement from airway tissue and therefore maintain an elongated shape until tissue growth has anchored the implant (e.g. to create a biological anchor). An end of the helix opposes wide section 10250 of the long term implant to thereby hold the long term implant in an elongated configuration. Compare the coils of the helix and the relative length of implant 10240 with bar 10244 in place along the coils in FIGS. 102 A, C with a foreshortened bioerodable helix, the unwound coils 10221 and relative length of implant 10220 without a support strut shown in FIG. 102B. The bioerodable portion in implant 120 did not resist a compressive force from the long term implant, and the long term implant prematurely shortened. A bar may connect essentially all of the coils of a helical portion together, as shown in FIG. 102A, C. Additionally, a second (or more than two) bar(s) may be placed along the helix to create additional regions of reduced flexibility, provide additional support, and reduce relative rotational movement of the helix.

Although any two (or more) coils may be connected by a bar, it may be especially useful to couple end coils, such as coils 10246, 10248, as shown in FIG. 102C to prevent rotation of the helix around the axis while maintaining device flexibility. In some embodiments, a first bar may be placed at a first end of a helix to connect a first set of end coils, and a second bar may be placed at a second end of a helix to connect a second set of end coils. In another embodiment, an additional bar(s) may be placed along two non-end coils to provide additional support, such as for backup in case one of the end bars breaks prematurely.

A support structure between two (or more) portions of a bioerodable material may be any material that provides support (e.g. creates regions of lesser flexibility) and may be any shape (e.g. a cylinder, a sphere, a straight bar, a wavy bar, a serpentine ribbon, etc.). A bar or other joining or support material may be connected with the bioerodable material using any means (e.g. heat or mechanical). The additional joining material may be the same material as the bioerodable portion or may be a different material.

A bioerodable material that is coupled to itself may be any shape that is able to maintain a shape of the long term implant and/or resist a compressive force (or maintain a tensioned force) from the long term implant. As shown in implant system 10300 in FIG. 103 A, B, additional joining material 10304 may couple two long sides 10302, 10303 of C-shaped or cuff shaped material 10307 to hold long-term elongate implant 10308 in an elongated position. A C-shaped or cuff shaped material may be easy to manufacture and may be easy to place (e.g. snap) over an elongated implant, and the additional joining material may hold the C-shaped material in place (e.g. prevent the cuff from falling off the elongated implant) so that it is able to resist a compressive force from wide section 10306 to thereby place or hold long term implant 10308 in an elongated shape. Although shown as a continuous material, a C-shaped bioerodable material may have any number of holes or open spaces. Holes or open spaces may improve device flexibility prior to bioerosion compared with a solid structure, and/or may allow better penetration of a body fluid. This may allow better timing of device erosion which in turn may influence both device anchoring and device function. In some embodiments, a bioerodable structure may be essentially a cylindrical structure that envelops an axis or envelopes part of an axis of a long term implant portion. A cylindrical structure (e.g. an open ended tube) may be continuous or may include open spaces (e.g. holes, slots). A bioerodable implant described herein, including a cylindrically shaped bioerodable implant, may be made by any means and may be connected with a long term implant using any methods or any means. In some embodiments, a portion of a bioerodable implant may be placed over a long term implant and then fastened in place (e.g. In FIG. 103, joining material 10304 in FIGS. 103 A-B may extend along the length of sides 10302, 10303 to form a cylindrical structure. Alternatively, a bioerodable implant may be manufactured as an extruded tube or may be formed using a mold, and after forming may be placed over the long term implant.

Any method or structure that allows the bioerodable portion to maintain a long term implant in a desired shape for a desired period of time may be used. A bioerodable material may be coupled to itself using a mechanical joining to hold two (or more) portions of bioerodable material together. Any form of mechanical joining may be used (e.g. forming a crimp, mating complementary portions together).

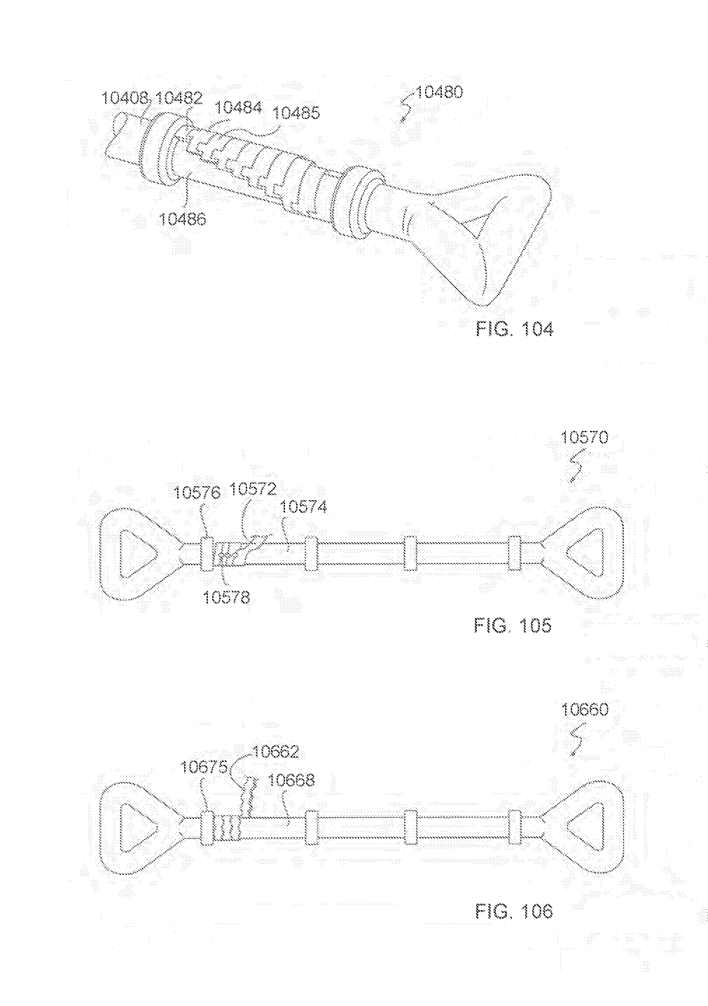

In one embodiment, a bioerodable portion may be coupled with one or more other bioerodable portions. Two C-shaped bioerodable pieces, each of which wraps partially around an axis (e.g. a long term implant portion) may be coupled (e.g. bridged) to one another. More than two (e.g. a series) of C-shaped or other shaped bioerodable pieces may be coupled together as shown in FIG. 104 to form system 10480 with a modular, mechanically interlocking bioerodable portion around long term implant 10408. Each piece may wrap a short distance (e.g. about a quarter of the way), halfway, or more than halfway (e.g. three-quarters of the way to form a C shape or all the way) around an axis, such as an elongate long term implant axis. The pieces may have any conformation and may have features (e.g. snap fit, lock and key,) to lock two or more than two pieces together and/or to lock or otherwise connect a piece with a long term implant portion. In one embodiment, the C-shaped (or other shaped) bioerodable pieces may be lined up (e.g. to substantially form a cuff shape that is open along one side similar to the cuff in FIG. 103 A, B) or bioerodable portions 10482, 10484, 10485 may be offset from one another relative to an elongate axis as shown in FIG. 104. Offset shapes may provide ease of assembly and/or may provide better (overall or local) implant flexibility, due to the presence of an open space, such as space 186 configured to allow implant bending. Having a space, such as space 10486, may also minimize an amount of biodegradable material present in a device, which may in turn minimize or prevent a side effect (such as inflammation) after a device system has been implanted in a body and the bioerodable portion has bioeroded.

One aspect of the invention provides a method of manufacturing a bioerodable implant including the steps of wrapping a bioerodable material at least partway around an axis to create a wound bioerodable implant, the bioerodable implant having two points, and coupling the two points to each other. See, for example, FIG. 101. Any biocompatible, bioerodable material can be used, including any described elsewhere in this application or known in the art.

Another aspect of the invention provides a method of manufacturing an implant system, the implant having an elongate (or resilient) implant body and a bioerodable support material configured to hold the elongate (or resilient) implant body in a first, elongate shape, including the steps of wrapping the bioerodable support material at least partway around the implant body, the bioerodable support material having two points on it, and coupling the two points with each other to create a coupled bioerodable support material.

In one embodiment, a thin strand of a polymer may be wrapped around an axis to create a helix and the helix may be coupled to itself. In one embodiment, a polymer may be based on lactic acid and/or glycolic (e.g. poly(lactic acid) or poly(DL-lactic-co-glycolic acid)) or any of the materials listed above or known in the art. The method may further include applying an expansive force to the elongate long term implant with the bioerodable material to thereby place or hold the long term implant in an initial shape, as shown in FIG. 101. A coupling step may include attaching (e.g. by applying an adhesive, by applying an other chemical (such as a polymer initiator), or by supplying an energy source) to two points on a bioerodable material to create a support strut. FIG. 101 shows applicator 10134 applying an activator (e.g. an adhesive, another chemical or an energy) to thereby fuse two points on the bioerodable material. Coupling the bioerodable may include heating the bioerodable material to melt the two points together. A bioerodable material may additionally (or instead) be coupled with a long term implant portion.

Another aspect of the invention provides a method of manufacturing an implant system, the implant having an elongate, resilient, long term implant body and a bioerodable (support) material configured to hold the resilient implant body in a first, elongate shape, the method including the steps of wrapping the bioerodable support material at least partway around the implant body (or at least partially enveloping the elongate implant body with a bioerodable support material), the bioerodable support material having two points on it; and coupling the two points with each other to create a coupled bioerodable support material.

In one embodiment, a ribbon like bioerodable material 10572 is wrapped at least partway around axis 10574, as shown in FIG. 105. The ribbon may overlap on itself. Two points on the ribbon-like material may be coupled to create a first bridge 10578 which has less flexibility than other points on the ribbon-like material. The bridge may be created by chemically joining two loops of the ribbon, by adding a new (bioerodable) structure between the two loops, or by mechanically interlocking the loops. Other bridges may be made between other loops of the ribbon-like material. In other embodiments, each loop may be coupled with one (or more than one) other loop(s). An end of the ribbon may abut wide region 10576 of the long term implant to thereby place (and/or hold) implant 10570 into a first shape. The ribbon-like material may have smooth edges or may have shaped edges. Shaped edges may be configured to couple, or mechanically connect (e.g. interlock). FIG. 106 shows implant 10660 with ribbon-like material 10662 being wound around long term implant 10668. Adjacent sections of ribbon-like material may mate to hold ribbon-like material 10662 in place. Any features that are able to hold the ribbon-like material together (and/or prevent it from unwinding relative to the long term implant) can be used. For example, the features may be tongue and groove or lock and key. A ribbon-like material may provide an expansive force to a long term implant portion; for example to wide section 10675.

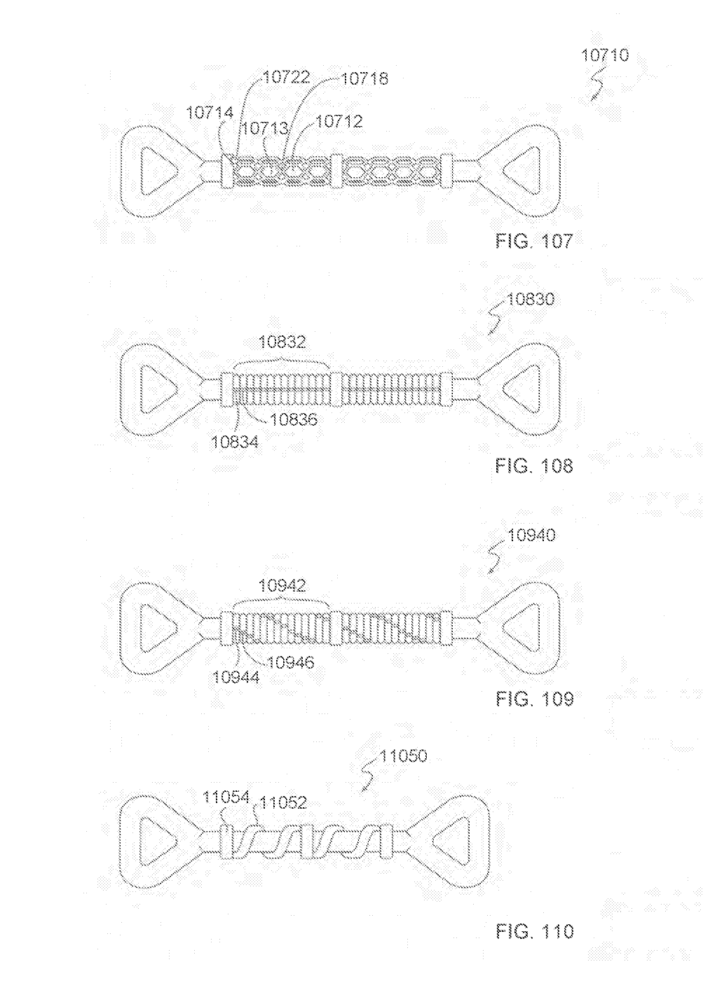

FIG. 107 shows another embodiment of an implant system with a bioerodable material configured to hold a long term implant in an initial or first shape. The bioerodable material may include a mesh or series of loops (e.g. a stent) that envelop or hold a long term implant to create implant system 10710. The loops may wrap all the way around the long term implant or may wrap partially around. Loops 10712, 10713 may be coupled with each other to create bridge 10718 of relatively lesser flexibility relative compared with the rest of the loop(s).

Alternatively, or additionally to being coupled to itself, the bioerodable material may be coupled with the long term implant. The bioerodable material may be coupled with the long term implant using any method or any material(s). The coupling may serve to hold the bioerodable material in a desired shape or configuration. A bioerodable material and a long term implant may be coupled using any chemical or mechanical means, including any described elsewhere in this application. As shown in FIG. 107, they may be coupled using corresponding mating structures 10714, 10722.

The bioerodable material may have regions of different flexibility. The regions may hold or help hold the bioerodable material (and the long-term elongate implant) in a preferred shape. FIGS. 108 and 109 show implants with bioerodable material having regions of differing flexibility. FIG. 108 shows implant system 10830 with a bioerodable spring coiled around a long term implant. The coils of the spring have regions 10834, 10836 that are less flexible than other portions (the rest) of the coils of bioerodable portion 10832. For example, these regions may be thicker, wider, or may include a different material with different resiliency (e.g. different flexibility). Any number of regions of lesser flexibility may be present on a coil. For example, each coil may have a second region of lesser flexibility on the coil (e.g. half-way around the coil) such that each coil has two regions of lesser flexibility (hinges) that control (e.g. prevents or reduces) a rotation or other movement of the coil. FIG. 109 shows implant 10940, which is similar to the device shown in FIG. 108, but regions of lesser flexibility 10944, 10946 in bioerodable portion 10942 are staggered relative to one another. Staggering a region having a lesser flexibility relative to another region of lesser flexibility may prevent the coil from unwinding while simultaneously allowing the coil (spring) to bend in various directions and to accommodate motion of the airway tissue (e.g. physiological movements, such as eating, breathing, or speaking).

The bioerodable material may have one or more than one (a plurality) of coils that wrap around a long axis (e.g. around a long-term elongate implant axis) no times (e.g. be a straight bar or a curve bar), or may wrap around the axis up to 1 time, up to 2 times, up to 3 times, up to 4 times, up to 5 times, up to 10 times, up to 20 times, up to 30 times, up to 40 times, or more than 40 times. FIG. 110 shows an embodiment of implant 11050 in which bioerodable material 11052 winds around (at least partially envelopes) the long term implant one and a half times. The bioerodable material may partially envelop the long term implant portion (as shown in FIG. 110) or may almost completely or may completely envelop the long term implant portion. The bioerodable material may be coupled with a long term implant at wide region 11054 to create a region of lesser flexibility on the bioerodable material.

The long term implant portion may have none, one or more than one wide portions that separate narrow portions. The wide portion may have a tensioned configuration and may provide a compressive force to the bioerodable portion such that the bioerodable portion holds the long-term implant portion in a preferred shape. FIG. 111 shows implant 11160 with two wide portions and a single spring 11162. The single spring (helix) partially envelops a narrow portion and is coupled with itself at two points at bridge 11168 near an end of the helix and at bridge 11166 near a central portion of the helix. In addition, or instead, the spring may be coupled at the other end of the helix (coil), and/or may be coupled at one or more places along the middle portions of the spring. The bioerodable portion may be coupled with the long term implant though bridge 11164 to hold the long-term implant portion in a preferred shape. The bioerodable portion may be coupled with the long-term implant portion is any way.

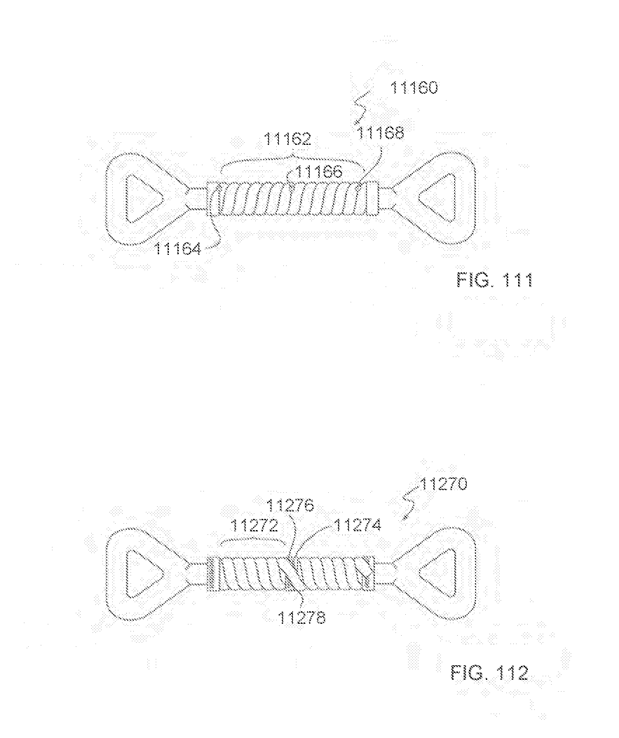

The long-term implant portion may couple with the bioerodable portion in any way (e.g. chemically, mechanically). FIG. 112 shows implant system 11270 in which wide portion 11274 of the long term, resilient, elongate implant includes channel 11276 configured to accept a cross portion 11278 of bioerodable material. A channel may grip or hold a cross portion of bioerodable material or the channel may provide a passage for a cross portion without gripping it. A cross portion may be configured along with section 11272 of a bioerodable helix to resist a compressive force or provide an expansive force to a long term implant portion, including to channel 11276. In one method of manufacturing an implant system with an implant having a resilient (or elongate) implant body including an implant body point and a bioerodable support material configured to hold the resilient (elongate) implant body, the method includes: wrapping the bioerodable support material at least partway around the implant body; and coupling the bioerodable support material with the implant body. The bioerodable support material may be coupled with the implant body using any means (e.g. chemical or mechanical). In one example, the first portion is a narrow portion and the second portion is a channel in a wide portion, the channel configured to hold the bioerodable material, and the bioerodable support material is passed through or along a surface of the channel. The implant body (channel) may hold the bioerodable material or the bioerodable material and the implant body may be fastened together, such as by a lock and key or a chemical coupling.

If different and multiple regions of coils are fused, a range of contraction times of the long term implant portion could be generated. Note that coil diameter also contributes to rate of contraction but may be secondary to coil fusion. In in vitro tests, the durometer of the material influenced contraction in the least significant manner. Nonetheless, taken together these parameters could be used to generate a matrix of physical properties that could influence the timing of the degradation of the coils, as well as match the bending properties of the device as a whole to tongue or other airway implant motion.

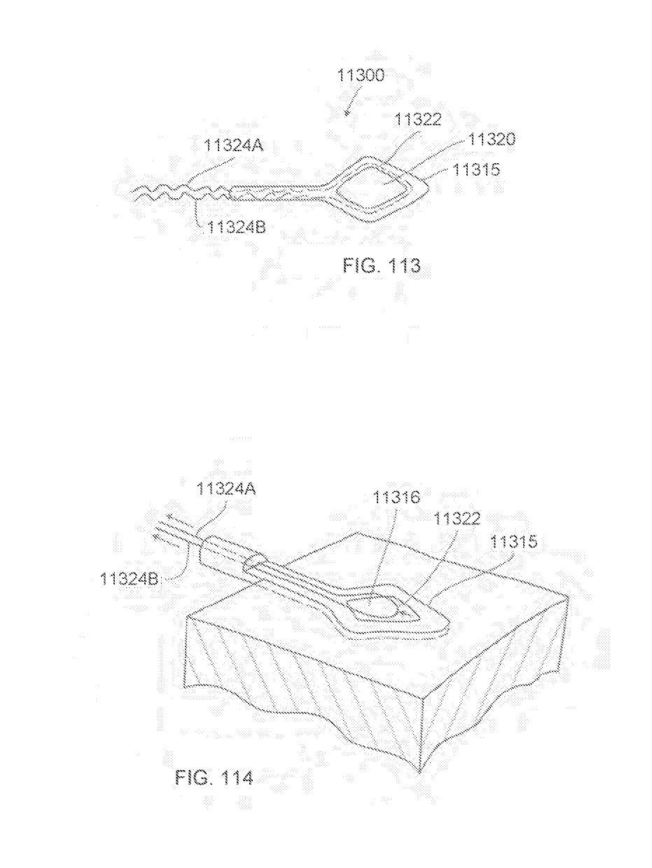

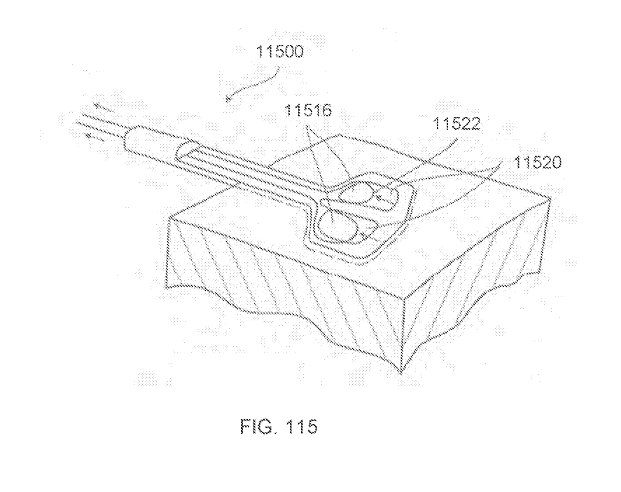

Any of the features described herein may be combined with any other features herein or as is known in the art. For example, any implant or any system may have a region(s) configured to be externally identifiable or visible or made externally identifiable or visible (e.g. by fluoroscopy), such as to a health care provider (physician) to aid in device placement, device tracking, and/or device removal. A region, such as wide sections 9908 shown in FIG. 99 A-D for example or at least part of anchor end 9904 may be platinum or other identifiable material. An implant or system may have a plurality of regions that are identifiable. In another example, any of the devices may be configured to be easily removable. FIGS. 113 and 114 illustrate another embodiment of revisable OSA implant 11300 that includes at least one end with an encircling portion indicated at 11315 that encircles or surrounds a tissue plug 11316 that grows through an opening 11320. In one embodiment, the implant carries a cut wire 11322 that extends in a loop with first and second wire ends 11324A and 11324B extending through one or more passageways in the implant. The cut wire 11322 can be embedded in the surface of the implant surrounding the opening 11320. As can be seen in FIG. 114, the looped cut wire 11322 can be pulled proximally to cut the tissue plug 11316 which then will free the implant from its attachment. In FIG. 113, it can be seen that the cut wire ends 11324A and 11324B can have a serpentine configuration in the medial portion of the implant so as to not interfere with the tensioning and relaxation of the elastomeric medial implant portion during its use. When the cut wire is accessed and pulled relative to the implant 11300, the tissue plug 11316 can be cut. It should be appreciated that other tools (not shown) may be used to stabilize the implant when actuating the cut wire as in FIG. 114. The cut wire 11322 can be any form of fine wire, or abrasive wire or a resistively heated wire coupled to an electrical source (not shown).

FIG. 115 depicts another revisable OSA implant 11500 that is similar to that of FIGS. 113-114 with the cut wire 11520 configured to cut a plurality of tissue plugs 11516 that have grown through openings 11520 within an encircling end portion of the implant body.

The devices may alternatively, or additionally, have reinforced anchor portions. The reinforced anchor portions may allow tissue to grow on or through an anchor portion and may serve to better anchor a device in place. The reinforced anchor portions may help hold an implant in place and/or may keep an implant from undergoing undesired stretching.

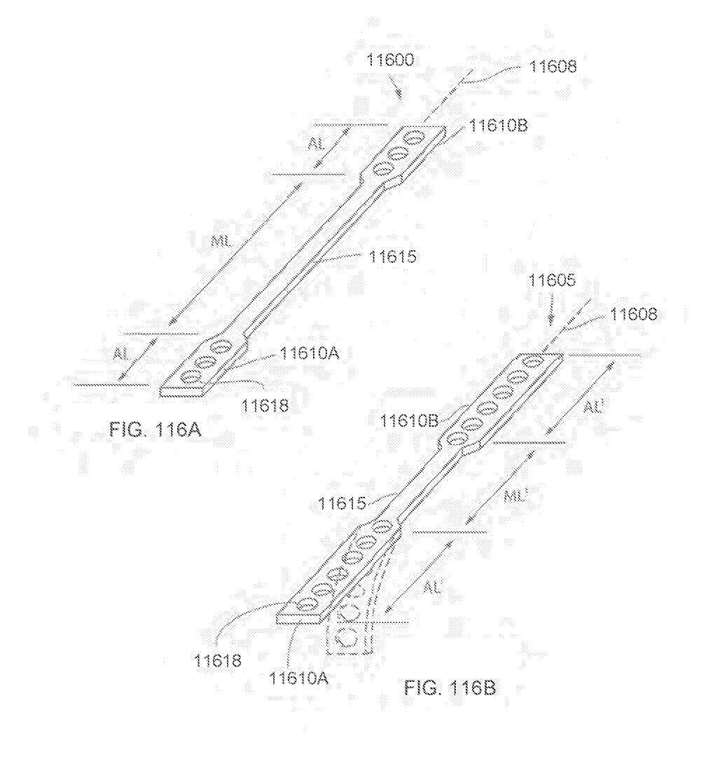

FIGS. 116A and 116B further illustrate that the anchor portion's axial length of AL (or AL′) can have a selected relationship to the medial portion's axial length ML (or ML′), and thus the overall implant length which is dependent on the desired amount of axial retraction forces applied by the implant. For example, in FIG. 116A, in one embodiment of an implant 11600 with axis 11608, each anchoring end 11610A, 11610B length AL can be 15% of the overall length of the implant which has a medial portion 11615 configured to apply a retraction force of 3.0 Newtons. FIG. 116B depicts another embodiment of an implant 11605 with axis 11608 and wherein each anchoring end 11610A, 11610B length AL′ can be 35% of the overall length of the implant and the medial portion 11615 with length ML′ can still be configured to apply a retraction force of 3.0 Newtons. In this embodiment, the design in FIG. 116B may be preferred because of the increased anchoring length, which would decrease the likelihood of tissue remodeling over time.

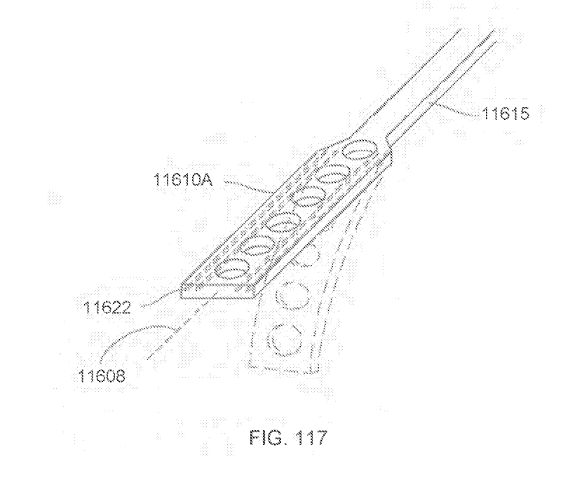

Referring to FIG. 117, it can be seen that an anchoring end portion 11610a of an OSA implant is made axially inelastic by means of non-stretchable reinforcing filaments or elements 11622 embedded therein. Such filaments 11622 can be an inelastic, flexible polymer (e.g., Kevlar®, or polyester), metal wires (e.g. stainless steel, NiTi), carbon fiber or the like. The filaments 11622 can be substantially linear elements or can be knit, woven, non woven, or braided structures as in known in the art. In another embodiment, the anchoring end portion may be made of a non-stretchable material without the addition of reinforcing filaments or elements. As can be understood from FIG. 117, the end portion 11610a is thus axially inelastic but is still flexible and twistable relative to axis 1008.

The devices described herein may be combined with other device features, including, but not limited to those described in U.S. Pat. No. 8,167,787, U.S. 2011/0144421, U.S. 2011/0226262, and U.S. patent application Ser. No. 13/308,449 to Gillis et al. filed Nov. 30, 2011.

Any of the devices or systems described herein may be configured to substantially hold the bioerodable material and/or may be configured to hold the long term implant in an initial (e.g. a first or a non-final) shape or configuration for less than 16 weeks (e.g. between 2 and 6 weeks, between 3 and 5 weeks, or for less than 1 week, less than 2 weeks, less than 3 weeks, less than 4 weeks, less than 5 weeks, less than 6 weeks, less than 7 weeks, or less than 8 weeks) when exposed to a body fluid or a saline solution. A body fluid that the device may be exposed to may be, for example, blood, interstitial fluid, lymph, mucus, nasal exudate or discharge, and/or saliva. A saline solution may be any saline solution, including a buffered saline solution. In one particular example, it is 0.1 M saline (0.1 M sodium chloride). After exposure to a body fluid for a sufficient period of time, an implant may take on a second, final, or therapeutic shape or configuration.

### EXAMPLES

### Example 1. Implant Contraction Accelerated Testing

An in vitro test system was developed to demonstrate the fatigue behavior of the restricting coils and simulate the expected motion after implantation. While not wishing to be limited to any theory, it is thought that the characteristic motions of the coiled implant when implanted are initially multiplanar bending. While some stretching of the device may occur, contraction is substantially prevented until the supporting coils degrade.

In order to evaluate the relative performance of types of coils, sets of implants with different durometer silicone cores and with coils that were either fused or open-ended within segments were rested. Coils with diameters of 0.009″ or 0.013″ were tested in 0.1M saline at 37° C. in a 20 L bath. The coils were made to oscillate by fixing one end and placing the body of the implant in a moving stream such that bending occurred at an approximate frequency of 2 Hz with a randomly oriented 15 degree to 30 degree bending motion. Bacterial growth was inhibited by addition of 0.01% sodium azide. Solutions were replaced each week and refreshed daily to replace fluid lost by evaporation. Temperature was maintained with the use of a submersible thermocouple regulated coil heater.

In general, regardless of the durometer of the silicone core tested, coil segments that were not fused at both ends began to unravel at their distal ends, causing a decrease in the stretched length of the implant from 36+/−1 mm to 21 to 27 mm by day 10 of the test. In contrast, implant systems with coils that were fused relaxed a stretched length of about 36 mm to about 34 mm by day 10.

Silicone cores with unfused coils contracted to 18 mm, which is their relaxed state, by day 15. For the fused coil materials tested, the rate of contraction depended little upon the durometer of the material or the diameter of the coil. The higher durometer (50 D) material tested with 0.013″ diameter fused coils contracted the least, contracting to only about 34 mm. 40 D material tested under the same conditions contracted to about 32 mm. This difference is small compared to the contraction levels observed in unfused coils and shows that although use of a higher durometer material can greatly increase the force on the coils, its effect is far less significant than is coil fusion. This suggests that the greatest impact on maintaining stretched implant length and avoiding early (premature) contraction was created by fusing coils.

### Example 2

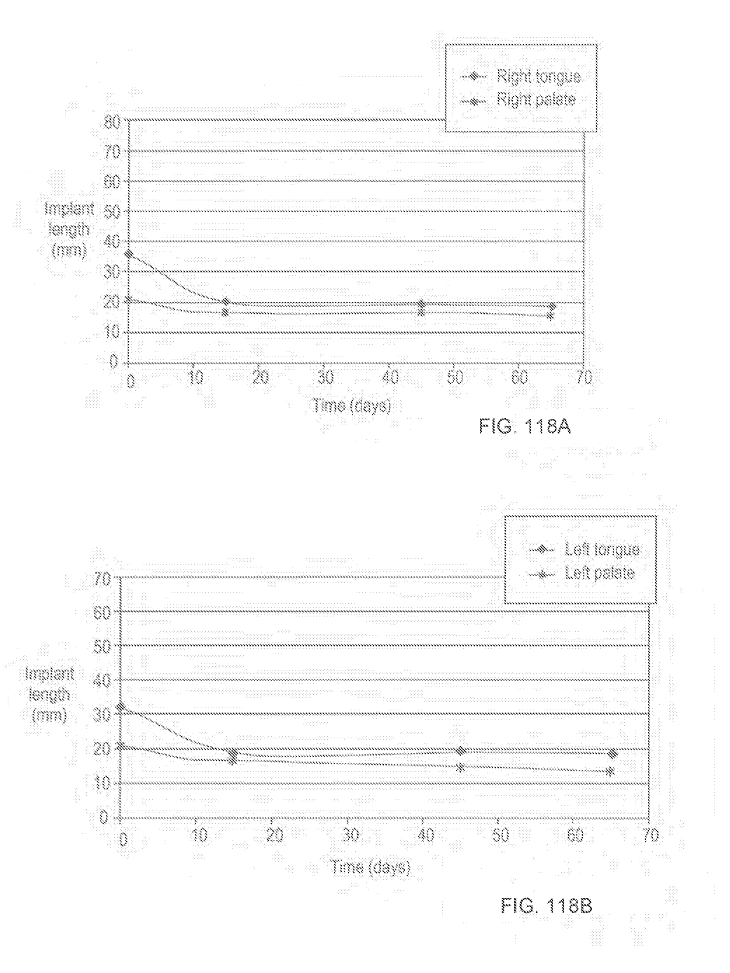

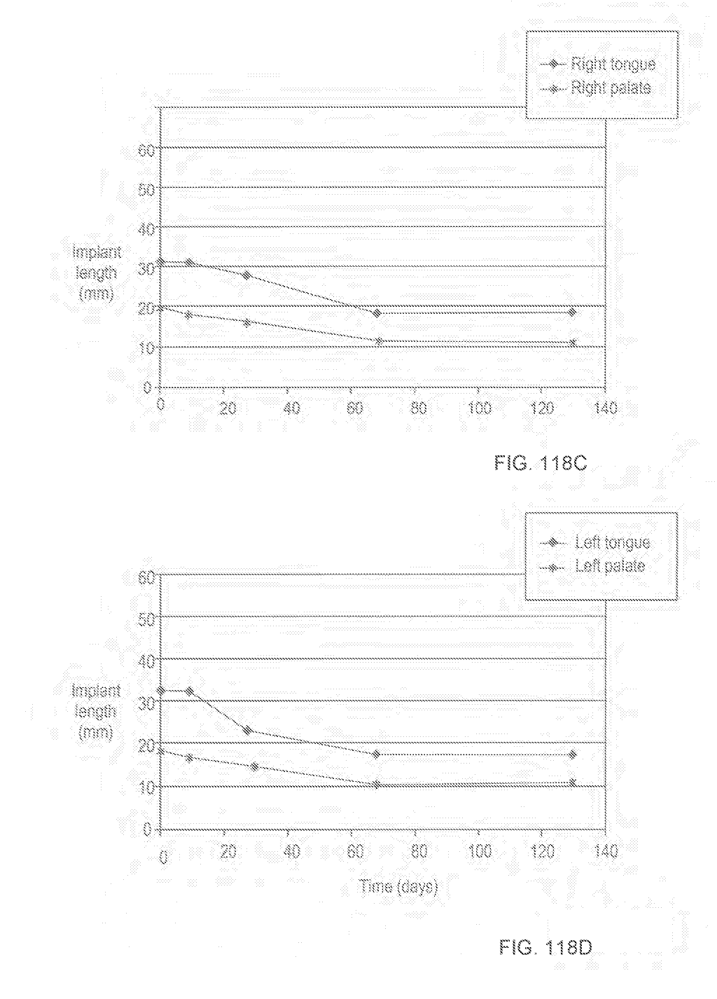

The effect of fusing implant coils together to prevent the coils from prematurely unwinding on the implant contraction rate was further tested using a canine animal model. Comparable implant systems having resilient, long term implants initially held in expanded shapes by coiled bioerodable implant material with (FIGS. 118 C-D) and without (FIGS. 118 A-B) fused coils were placed on both left and right sides of animals' tongues and soft palates and the bioerodable material bioeroded to allow the resilient, long term implants to foreshorten. The implant lengths were measured as a function of length of time since implantation. Noting that the time scales are different, as seen by the more gradually downward sloping curves going from the initial implant lengths (at time=0) to the equilibrium (foreshortened) implant lengths in the results shown with the fused coils placed in the right and left sides of the tongue (“Right tongue” and “Left tongue”, respectively) as shown FIGS. 118 C-D compared with the steeper, downwardly sloping lines obtained from the corresponding unfused implants shown in FIGS. 118 A-B, resilient implants with fused bioerodable coils placed in the tongue shortened more slowly and took a longer overall time to reach a (fully) foreshortened length than did resilient without fused bioerordable coils. Systems with fused coils took several weeks (e.g. more than 22 days and possibly as long as 25-40 days or more) to contract to about 20 mm, about the same implant length that systems having unfused coils reached by about 10-14 days after implantation. Comparing results of fused coil implant systems (with unfused coil implant systems in the tongue (FIGS. 118 A-D), bioerodable coil fusion eliminated about 80% of the amount of contraction (foreshortening) observed in the unfused coil system at 14 days. The rate of foreshortening was also slower in implant systems in the soft palate having fused coils compared with implant systems without fused coils (compare results for “Right palate” and “Left palate” in FIGS. 118 A-D).

The embodiments of implants shown in the figures above can be sized and shaped to conform to a treatment site in a patient's tongue, palate or other site in airway-interface tissue and to reside in an orientation and in a manner compatible with normal physiological function of the site. The overall dimensions may vary according to the full extent that human subjects vary in their anatomical dimensions, and thus the dimensions provided here are only an approximation for the purpose of illustration, and are not meant to be limiting. Any embodiment in its elongated state may typically be in the range of about 2 cm to about 10 cm in length in a releasably extended state, and the implant in a contracted state may be in the range of about 1 cm to about 6 cm in length. Testing shows there is an advantage to using these lengths.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

Unless defined otherwise, all technical terms used herein have the same meanings as commonly understood by one of ordinary skill in the art to which this invention belongs. Specific methods, devices, and materials are described in this application, but any methods and materials similar or equivalent to those described herein can be used in the practice of the present invention. While embodiments of the inventive device and method have been described in some detail and by way of exemplary illustrations, such illustration is for purposes of clarity of understanding only, and is not intended to be limiting.

Various terms have been used in the description to convey an understanding of the invention; it will be understood that the meaning of these various terms extends to common linguistic or grammatical variations or forms thereof. It will also be understood that when terminology referring to devices or equipment has used trade names, brand names, or common names, that these names are provided as contemporary examples, and the invention is not limited by such literal scope. Terminology that is introduced at a later date that may be reasonably understood as a derivative of a contemporary term or designating of a subset of objects embraced by a contemporary term will be understood as having been described by the now contemporary terminology.

While some theoretical considerations have been advanced in furtherance of providing an understanding of the invention the claims to the invention are not bound by such theory. Described herein are ways that embodiments of the invention may engage the anatomy and physiology of the airway, generally by opening the airway during sleep; the theoretical consideration being that by such opening of the airway, the implanted device embodiments alleviate the occurrence of apneic events. Moreover, any one or more features of any embodiment of the invention can be combined with any one or more other features of any other embodiment of the invention, without departing from the scope of the invention. Further, it should be understood that while these inventive methods and devices have been described as providing therapeutic benefit to the airway by way of intervention in tissue lining the airway, such devices and embodiments may have therapeutic application in other sites within the body, particularly luminal sites. Still further, it should be understood that the invention is not limited to the embodiments that have been set forth for purposes of exemplification, but is to be defined only by a fair reading of claims that are appended to the patent application, including the full range of equivalency to which each element thereof is entitled.

What is claimed is:

1. A method of treating an airway disorder comprising:
implanting at least first and second elongated implants in a tongue of a patient, wherein each of the first and second implants is configured to have a first, expanded configuration and a second, contracted configuration, wherein implanting comprises implanting the first and second implants having their first, expanded configurations, and wherein each implant has an anterior end in an anterior location and a posterior end in a posterior location in the patient's tongue and the posterior end locations are different vertical distances from a transverse plane of a patient.
